SEQUENCE LISTING (174 sequences)

SEQ ID NO: 1 - protein, 220 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 3 - protein, 181 aa, Homo sapiens
NSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVS

SEQ ID NO: 4 - protein, 7 aa, synthetic construct
DKTHTCP

SEQ ID NO: 5 - protein, 10 aa, synthetic construct
DKTHTCPPCP

SEQ ID NO: 6 - protein, 15 aa, synthetic construct
EPKSCDKTHTCPPCP

SEQ ID NO: 7 - protein, 760 aa, synthetic construct
MMDQARSAFSNLFGGEPLSYTRFSLARQVDGDNSHVEMKLGVDEEENTDNNTKANGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 8 - DNA, 16 bp, synthetic construct
  source 1..16: mol_type = other DNA
gcattctaatagcagc

SEQ ID NO: 9 - protein, 214 aa, synthetic construct
DIQMTQSPSSLSASVGDRVTITCRTSENIYNNLAWYQQKPGKSPKLLIYAATNLADGVPSRFSGSGSGTDYTLTISSLQPEDFATYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 446 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 446 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 12 - protein, 10 aa, synthetic construct
GYTFTNYWMH

SEQ ID NO: 13 - protein, 17 aa, synthetic construct
EINPINGRSNYAEKFQG

SEQ ID NO: 14 - protein, 9 aa, synthetic construct
ARGTRAMHY

SEQ ID NO: 15 - protein, 11 aa, synthetic construct
RTSENIYNNLA

SEQ ID NO: 16 - protein, 7 aa, synthetic construct
AATNLAD

SEQ ID NO: 17 - protein, 9 aa, synthetic construct
QHFWGTPLT

SEQ ID NO: 18 - protein, 214 aa, synthetic construct
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 446 aa, synthetic construct
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 450 aa, synthetic construct
QVTLRESGPALVKPTQTLTLTCTFSGFSLSTSGMSVGWIRQPPGKALEWLADIWWDDKKDYNPSLKSRLTISKDTSKNQVVLKVTNMEPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 21 - protein, 10 aa, synthetic construct
GYTFTSYWMH

SEQ ID NO: 22 - protein, 17 aa, synthetic construct
EINPTNGRTNYIEKFKS

SEQ ID NO: 23 - protein, 9 aa, synthetic construct
ARGTRAYHY

SEQ ID NO: 24 - protein, 11 aa, synthetic construct
RASDNLYSNLA

SEQ ID NO: 25 - protein, 7 aa, synthetic construct
DATNLAD

SEQ ID NO: 26 - protein, 9 aa, synthetic construct
QHFWGTPLT

SEQ ID NO: 27 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 28 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 29 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 30 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 31 - protein, 219 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 32 - protein, 219 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 33 - protein, 219 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 34 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 107 aa, synthetic construct
EIVMTQSPATLSLSPGERATLSCSASSSVSYMYWYQQKPGQAPRLLIYDTSNLASGIPARFSGSGSGTDFTLTISSLQPEDFAVYYCQQWSSYPPITFGQGTKVEIK

SEQ ID NO: 36 - protein, 115 aa, synthetic construct
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSS

SEQ ID NO: 37 - protein, 214 aa, synthetic construct
EIVMTQSPATLSLSPGERATLSCSASSSVSYMYWYQQKPGQAPRLLIYDTSNLASGIPARFSGSGSGTDFTLTISSLQPEDFAVYYCQQWSSYPPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 216 aa, synthetic construct
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPK

SEQ ID NO: 39 - protein, 11 aa, synthetic construct
SASSSVSMYWY

SEQ ID NO: 40 - protein, 13 aa, synthetic construct
RASQSVSSYLAWY

SEQ ID NO: 41 - protein, 7 aa, synthetic construct
DTSNLAS

SEQ ID NO: 42 - protein, 7 aa, synthetic construct
DASNLAT

SEQ ID NO: 43 - protein, 10 aa, synthetic construct
QQWSSYPPIT

SEQ ID NO: 44 - protein, 10 aa, synthetic construct
QQRSNWPPIT

SEQ ID NO: 45 - protein, 11 aa, synthetic construct
GYSITSDYAWN

SEQ ID NO: 46 - protein, 11 aa, synthetic construct
GYSITSDYAWG

SEQ ID NO: 47 - protein, 16 aa, synthetic construct
YMSYSGSTRYNPSLRS

SEQ ID NO: 48 - protein, 16 aa, synthetic construct
SMSYSGSTYYNPSLKS

SEQ ID NO: 49 - protein, 8 aa, synthetic construct
ARGWPLAY

SEQ ID NO: 50 - protein, 16 aa, synthetic construct
  VARIANT 1: note = X is Y or S
  VARIANT 9: note = X is R or Y; note = X is R or Y
  VARIANT 15: note = X is R or K
XMSYSGSTXYNPSLXS

SEQ ID NO: 51 - protein, 108 aa, synthetic construct
EIVLTQSPATLSLSPGERATLSCRASQSVSSYLAWYQQKPGQAPRLLIYDASNLATGIPARFSGSGSGTDFTLTISSLEPEDFAVYYCQQRSNWPPITFGQGTKVEIK

SEQ ID NO: 52 - protein, 214 aa, synthetic construct
EIVMTQSPATLSLSPGERATLSCSASSSVSYMYWYQQKPGQAPRLLIYDTSNLASGIPARFSGSGSGTDFTLTISSLQPEDFAVYYCQQWSSYPPITFGQGTKVEIKRTVAAPSVFIFPPSDEQLKSGTASVVCLLNNFYPREAKVQWCVDNALQSGNSQESVTEQDSKDSTYSLSSTLTLSKADYEKHKVYACEVTHQGLSSPVTKSFNRGEC

SEQ ID NO: 53 - protein, 218 aa, synthetic construct
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 54 - protein, 225 aa, synthetic construct
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 55 - protein, 228 aa, synthetic construct
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCP

SEQ ID NO: 56 - protein, 216 aa, synthetic construct
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPK

SEQ ID NO: 57 - protein, 218 aa, synthetic construct
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 58 - protein, 225 aa, synthetic construct
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 59 - protein, 228 aa, synthetic construct
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCP

SEQ ID NO: 60 - protein, 115 aa, synthetic construct
QVQLQESGPGLVKPSETLSLTCAVSGYSITSDYAWGWIRQPPGKGLEWIGSMSYSGSTYYNPSLKSRVTISVDTSKNQFSLKLSSVTAADTAVYYCARGWPLAYWGQGTLVTVSS

SEQ ID NO: 61 - protein, 216 aa, synthetic construct
QVQLQESGPGLVKPSETLSLTCAVSGYSITSDYAWGWIRQPPGKGLEWIGSMSYSGSTYYNPSLKSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPK

SEQ ID NO: 62 - protein, 215 aa, synthetic construct
EIVLTQSPATLSLSPGERATLSCRASQSVSSYLAWYQQKPGQAPRLLIYDASNLATGIPARFSGSGSGTDFTLTISSLEPEDFAVYYCQQRSNWPPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 108 aa, synthetic construct; identical to SEQ ID NO: 51

SEQ ID NO: 64 - protein, 215 aa, synthetic construct
EIVLTQSPATLSLSPGERATLSCRASQSVSSYLAWYQQKPGQAPRLLIYDASNLATGIPARFSGSGSGTDFTLTISSLEPEDFAVYYCQQRSNWPPITFGQGTKVEIKRTVAAPSVFIFPPSDEQLKSGTASVVCLLNNFYPREAKVQWCVDNALQSGNSQESVTEQDSKDSTYSLSSTLTLSKADYEKHKVYACEVTHQGLSSPVTKSFNRGEC

SEQ ID NO: 65 - protein, 218 aa, synthetic construct
QVQLQESGPGLVKPSETLSLTCAVSGYSITSDYAWGWIRQPPGKGLEWIGSMSYSGSTYYNPSLKSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 66 - protein, 225 aa, synthetic construct
QVQLQESGPGLVKPSETLSLTCAVSGYSITSDYAWGWIRQPPGKGLEWIGSMSYSGSTYYNPSLKSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 67 - protein, 228 aa, synthetic construct
QVQLQESGPGLVKPSETLSLTCAVSGYSITSDYAWGWIRQPPGKGLEWIGSMSYSGSTYYNPSLKSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCP

SEQ ID NO: 68 - protein, 216 aa, synthetic construct
QVQLQESGPGLVKPSETLSLTCAVSGYSITSDYAWGWIRQPPGKGLEWIGSMSYSGSTYYNPSLKSRVTISVDTSKNQFSLKLSSVTAADTAVYYCARGWPLAYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPK

SEQ ID NO: 69 - protein, 218 aa, synthetic construct
QVQLQESGPGLVKPSETLSLTCAVSGYSITSDYAWGWIRQPPGKGLEWIGSMSYSGSTYYNPSLKSRVTISVDTSKNQFSLKLSSVTAADTAVYYCARGWPLAYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 70 - protein, 225 aa, synthetic construct
QVQLQESGPGLVKPSETLSLTCAVSGYSITSDYAWGWIRQPPGKGLEWIGSMSYSGSTYYNPSLKSRVTISVDTSKNQFSLKLSSVTAADTAVYYCARGWPLAYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 71 - protein, 228 aa, synthetic construct
QVQLQESGPGLVKPSETLSLTCAVSGYSITSDYAWGWIRQPPGKGLEWIGSMSYSGSTYYNPSLKSRVTISVDTSKNQFSLKLSSVTAADTAVYYCARGWPLAYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCP

SEQ ID NO: 72 - protein, 324 aa, synthetic construct
GAAATTGTCCTCACCCAGAGCCCAGCCACTCTTTCCCTCTCTCCAGGGGAAAGAGCAACACTGAGTTGCCGGGCCTCCCAGAGTGTAAGTTCCTACCTCGCATGGTATCAACAGAAGCCTGGTCAAGCACCCCGGCTTCTGATTTACGATGCCAGCAATCTCGCCACCGGAATCCCCGCAAGGTTTTCAGGGAGCGGTTCAGGGACTGACTTTACCCTGACCATAAGCAGTCTTGAGCCTGAGGATTTTGCAGTGTATTACTGCCAACAGAGAAGTAACTGGCCCCCCATAACCTTTGGACAGGGGACTAAGGTGGAGATTAAA

SEQ ID NO: 73 - DNA, 324 bp, synthetic construct
  source 1..324: mol_type = other DNA
gagatcgtactcactcaaagtccagccaccctgtcactttctccaggagaacgcgccactctctcttgtcgggctagtcagagcgttagtagctatctcgcatggtatcaacaaaagcccggtcaggccccacgcctgttgatctatgatgccagtaacctggccaccggtattccagcccgtttctctggcagtggaagtggaactgacttcacactgaccatcagctccctcgagcccgaagattttgctgtgtattactgccagcaaagatccaactggcctccaatcactttcggccaaggaacaaaagtagagattaag

SEQ ID NO: 74 - DNA, 345 bp, synthetic construct
  source 1..345: mol_type = other DNA
caagtgcaactgcaggaaagtgggcctggtttggtcaaacccagtgaaacacttagtcttacttgcgctgtatctggatactctattacaagcgattatgcctgggggtggattcggcagccccctggaaaaggcttggagtggatcggttcaatgagttatagtggtagtacatactacaacccttccctgaagtctcgcgtcacaataagcgtagatacaagtaagaatcaattttccctgaaacttagcagtgtaactgccgctgataccgcagtctactattgtgccagaggatggcccctggcctactggggtcagggcaccctcgtgaccgtatcatca

SEQ ID NO: 75 - DNA, 345 bp, synthetic construct
  source 1..345: mol_type = other DNA
caggttcaacttcaagagtcaggcccaggcttggtgaagccctccgaaactttgagcttgacttgcgctgtgtccggttacagtataacctctgactatgcctgggggtggataaggcaacctcctggcaaaggattggaatggatcggttcaatgtcctactccggttctacatactataacccctccttgaagtcccgagtaactatcagtgtggacacttcaaagaatcaatttagtctgaaattgtcttcagtcaccgctgcagacactgctgtctattattgtgccaggggttggccccttgcttattggggccaggggaccctggtgacagtatcaagc

SEQ ID NO: 76 - protein, 220 aa, synthetic construct; identical to SEQ ID NO: 27

SEQ ID NO: 77 - protein, 220 aa, synthetic construct; identical to SEQ ID NO: 28

SEQ ID NO: 78 - protein, 220 aa, synthetic construct
PCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 79 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPCPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 80 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLCLSPGK

SEQ ID NO: 81 - protein, 220 aa, synthetic construct; identical to SEQ ID NO: 29

SEQ ID NO: 82 - protein, 220 aa, synthetic construct; identical to SEQ ID NO: 30

SEQ ID NO: 83 - protein, 220 aa, synthetic construct
PCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 84 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSCPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 85 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLCLSPGK

SEQ ID NO: 86 - protein, 220 aa, synthetic construct; identical to SEQ ID NO: 34

SEQ ID NO: 87 - protein, 220 aa, synthetic construct
PCPAPEAAGGPSVFLFPPKPKDTLMISRTPEVTCVVVDVSHEDPEVKFNWYVDGVEVHNAKCKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 88 - protein, 220 aa, synthetic construct
PCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 89 - protein, 220 aa, synthetic construct
PCPAPEAAGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 90 - protein, 220 aa, synthetic construct
PCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPCPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 91 - protein, 330 aa, synthetic construct
CSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 92 - protein, 220 aa, synthetic construct
PCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 93 - protein, 220 aa, synthetic construct
PCPAPEAAGGPSVFLFPPKPKDTLMISRTPEVTCVVVDVSHEDPEVKFNWYVDGVEVHNAKCKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 94 - protein, 220 aa, synthetic construct
PCPAPEAAGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 95 - protein, 220 aa, synthetic construct
PCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 96 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGCPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 97 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLCLSPGK

SEQ ID NO: 98 - protein, 220 aa, synthetic construct
PCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALGCPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 99 - protein, 330 aa, synthetic construct
CSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 100 - protein, 107 aa, synthetic construct
RTVAAPSVFIFPPSDEQLKSGTASVVCLLNNFYPREAKVQWCVDNALQSGNSQESVTEQDSKDSTYSLSSTLTLSKADYEKHKVYACEVTHQGLSSPVTKSFNRGEC

SEQ ID NO: 101 - protein, 213 aa, synthetic construct
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 220 aa, synthetic construct
EVQLVESGGGLVQPGGSLRLSCAVSGFSLTTYGVDWVRQAPGKGLEWVGIIWGDGTTHYHSALISRFTISKDNSKNTVYLQMNSLRAEDTAVYYCAKPGGASWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 103 - protein, 214 aa, synthetic construct
DIQMTQSPSSLSASVGDRVTITCKASQLVGTNVAWYQQKPGKSPKALFYSASYRNQGVPSRFSGSGSGTDFTLTISSLQPEDFATYFCQHYNSYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 220 aa, synthetic construct; identical to SEQ ID NO: 102

SEQ ID NO: 105 - protein, 214 aa, synthetic construct
DIQMTQSPSSLSASVGDRVTITCKASQFVGTAVAWYQQKPGKSPKALFYSASYRNEGVPSRFSGSGSGTDFTLTISSLQPEDFATYFCQHYNSYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 240 aa, synthetic construct
EVQLVESGGGLVQPGGSLRLSCAVSGFSLTTYGVDWVRQAPGKGLEWVGIIWGDGTTHYHSALISRFTISKDNSKNTVYLQMNSLRAEDTAVYYCAKPGGASWGQGTLVTVSSGGGGSGGGGSGGGGSDIQMTQSPSSLSASVGDRVTITCKASQLVGTNVAWYQQKPGKSPKALFYSASYRNQGVPSRFSGSGSGTDFTLTISSLQPEDFATYFCQHYNSYPFTFGQGTKVEIKEPKSS

SEQ ID NO: 107 - protein, 240 aa, synthetic construct
EVQLVESGGGLVQPGGSLRLSCAVSGFSLTTYGVDWVRQAPGKGLEWVGIIWGDGTTHYHSALISRFTISKDNSKNTVYLQMNSLRAEDTAVYYCAKPGGASWGQGTLVTVSSGGGGSGGGGSGGGGSDIQMTQSPSSLSASVGDRVTITCKASQFVGTAVAWYQQKPGKSPKALFYSASYRNEGVPSRFSGSGSGTDFTLTISSLQPEDFATYFCQHYNSYPFTFGQGTKVEIKEPKSS

SEQ ID NO: 108 - protein, 448 aa, synthetic construct
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 219 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 221 aa, synthetic construct
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 111 - protein, 219 aa, synthetic construct; identical to SEQ ID NO: 109

SEQ ID NO: 112 - protein, 118 aa, synthetic construct
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSS

SEQ ID NO: 113 - protein, 112 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQSTHVPWTFGQGTKVEIK

SEQ ID NO: 114 - protein, 10 aa, synthetic construct
GYSFTNYWLG

SEQ ID NO: 115 - protein, 17 aa, synthetic construct
DIYPGGDYPTYSEKFKV

SEQ ID NO: 116 - protein, 9 aa, synthetic construct
SGNYDEVAY

SEQ ID NO: 117 - protein, 16 aa, synthetic construct
RSSQSLVHSNGNTYLH

SEQ ID NO: 118 - protein, 7 aa, synthetic construct
KVSNRFS

SEQ ID NO: 119 - protein, 9 aa, synthetic construct
SQSTHVPWT

SEQ ID NO: 120 - protein, 448 aa, synthetic construct
EVQLVQSGAEVKKPGESLKISCKGSGYSFMNYWLGWVRQMPGKGLEWMGDIYPGGDYPTYSEKFKVQVTISADKSISTAYLQLSSLKASDTAMYYCARSGNYDEVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 219 aa, synthetic construct; identical to SEQ ID NO: 109

SEQ ID NO: 122 - protein, 221 aa, synthetic construct
EVQLVQSGAEVKKPGESLKISCKGSGYSFMNYWLGWVRQMPGKGLEWMGDIYPGGDYPTYSEKFKVQVTISADKSISTAYLQLSSLKASDTAMYYCARSGNYDEVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 123 - protein, 219 aa, synthetic construct; identical to SEQ ID NO: 109

SEQ ID NO: 124 - protein, 118 aa, synthetic construct
EVQLVQSGAEVKKPGESLKISCKGSGYSFMNYWLGWVRQMPGKGLEWMGDIYPGGDYPTYSEKFKVQVTISADKSISTAYLQLSSLKASDTAMYYCARSGNYDEVAYWGQGTLVTVSS

SEQ ID NO: 125 - protein, 112 aa, synthetic construct; identical to SEQ ID NO: 113

SEQ ID NO: 126 - protein, 10 aa, synthetic construct
GYSFMNYWLG

SEQ ID NO: 127 - protein, 17 aa, synthetic construct
DIYPGGDYPTYSEKFKV

SEQ ID NO: 128 - protein, 9 aa, synthetic construct
SGNYDEVAY

SEQ ID NO: 129 - protein, 16 aa, synthetic construct
RSSQSLVHSNGNTYLH

SEQ ID NO: 130 - protein, 7 aa, synthetic construct
KVSNRFS

SEQ ID NO: 131 - protein, 9 aa, synthetic construct
SQSTHVPWT

SEQ ID NO: 132 - protein, 229 aa, synthetic construct
QSMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 215 aa, synthetic construct
AIQLTQSPSSLSASVGDRVTITCRASQSISSYLAWYQQKPGKAPKLLIYRASTLASGVPSRFSGSGSGTDFTLTISSLQPEDFATYYCQQNYASSNVDNTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 134 - protein, 10 aa, synthetic construct
GFSLSSYAMS

SEQ ID NO: 135 - protein, 16 aa, synthetic construct
YIWSGGSTDYASWAKS

SEQ ID NO: 136 - protein, 17 aa, synthetic construct
RYGTSYPDYGDASGFDP

SEQ ID NO: 137 - protein, 11 aa, synthetic construct
RASQSISSYLA

SEQ ID NO: 138 - protein, 7 aa, synthetic construct
RASTLAS

SEQ ID NO: 139 - protein, 12 aa, synthetic construct
QQNYASSNVDNT

SEQ ID NO: 140 - protein, 446 aa, synthetic construct; identical to SEQ ID NO: 10

SEQ ID NO: 141 - protein, 446 aa, synthetic construct; identical to SEQ ID NO: 11

SEQ ID NO: 142 - protein, 214 aa, synthetic construct; identical to SEQ ID NO: 9

SEQ ID NO: 143 - protein, 219 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 144 - protein, 214 aa, synthetic construct; identical to SEQ ID NO: 9

SEQ ID NO: 145 - protein, 116 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSS

SEQ ID NO: 146 - protein, 107 aa, synthetic construct
DIQMTQSPSSLSASVGDRVTITCRTSENIYNNLAWYQQKPGKSPKLLIYAATNLADGVPSRFSGSGSGTDYTLTISSLQPEDFATYYCQHFWGTPLTFGGGTKVEIK

SEQ ID NO: 147 - protein, 10 aa, synthetic construct
GYTFTNYWMH

SEQ ID NO: 148 - protein, 17 aa, synthetic construct
EINPINGRSNYAEKFQG

SEQ ID NO: 149 - protein, 9 aa, synthetic construct
ARGTRAMHY

SEQ ID NO: 150 - protein, 11 aa, synthetic construct
RTSENIYNNLA

SEQ ID NO: 151 - protein, 7 aa, synthetic construct
AATNLAD

SEQ ID NO: 152 - protein, 9 aa, synthetic construct
QHFWGTPLT

SEQ ID NO: 153 - protein, 243 aa, synthetic construct
QVQLQQWGAGLLKPSETLSLTCAVYGGSFSGYYWTWIRQPPGKGLEWIGEFIHSGSTNYNPSLKSRVTISVDTSKNQFSLKLSSVTAADTAVYFCARGSMDSSGFYSFDFWGQGTLVTVSSGTEGKSSGSGSESKSTSYEVTQPPSVSVSPGQTASITCSGHKLGDKFASWYQQKPGQSPVLVIYQDRKRPSGIPERFSGSNSGNTATLTISGTQAMDEADYYCQTWYSSTVIFGGGTKLTVL

SEQ ID NO: 154 - protein, 8 aa, synthetic construct
GGSFSGYY

SEQ ID NO: 155 - protein, 7 aa, synthetic construct
FIHSGST

SEQ ID NO: 156 - protein, 15 aa, synthetic construct
ARGSMDSSGFYSFDF

SEQ ID NO: 157 - protein, 6 aa, synthetic construct
KLGDKF

SEQ ID NO: 158 - protein, 6 aa, synthetic construct
QDRKRP

SEQ ID NO: 159 - protein, 9 aa, synthetic construct
QTWYSSTVI

SEQ ID NO: 160 - protein, 244 aa, synthetic construct
EVQLLESGGGLVQPGGSLRLSCADSGFTFSSYAMNWVRQAPGKGLEWVSGISGSGGHTYYADSVKGRFTVSRDNSKNTLYLQMNSLRAEDTAVYYCAREGYDSSGYNPFDYWGQGTQVTVSSGTEGKSSGSGSESKSTSYELTQPPSVSVSPGQTASITCSGDKLGDKYASWYQQKPGQSPVLVIYQDSKRPSGIPERFSGSNSGNTATLTISGTQAMDEADYYCQAWDSSTVVFGGGTKLTVL

SEQ ID NO: 161 - protein, 11 aa, synthetic construct
GFTFSSYAMNW

SEQ ID NO: 162 - protein, 8 aa, synthetic construct
ISGSGGHT

SEQ ID NO: 163 - protein, 15 aa, synthetic construct
AREGYDSSGYNPFDY

SEQ ID NO: 164 - protein, 7 aa, synthetic construct
KLGDKYA

SEQ ID NO: 165 - protein, 7 aa, synthetic construct
QDSKRPS

SEQ ID NO: 166 - protein, 9 aa, synthetic construct
QAWDSSTVV

SEQ ID NO: 167 - protein, 585 aa, synthetic construct [sequence omitted]

SEQ ID NO: 168 - protein, 584 aa, synthetic construct
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 169 - protein, 824 aa, synthetic construct
EVQLVESGGGLVQPGGSLRLSCAVSGFSLTTYGVDWVRQAPGKGLEWVGIIWGDGTTHYHSALISRFTISKDNSKNTVYLQMNSLRAEDTAVYYCAKPGGASWGQGTLVTVSSGGGGSGGGGSGGGGSDIQMTQSPSSLSASVGDRVTITCKASQNVGTNVAWYQQKPGKSPKALFYSASYRNAGVPSRFSGSGSGTDFTLTISSLQPEDFATYFCQHYNSYPFTFGQGTKVEIKGGGGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 170 - protein, 825 aa, synthetic construct
EVQLVESGGGLVQPGGSLRLSCAVSGFSLTTYGVDWVRQAPGKGLEWVGIIWGDGTTHYHSALISRFTISKDNSKNTVYLQMNSLRAEDTAVYYCAKPGGASWGQGTLVTVSSGGGGSGGGGSGGGGSDIQMTQSPSSLSASVGDRVTITCKASQNVGTNVAWYQQKPGKSPKALFYSASYRNAGVPSRFSGSGSGTDFTLTISSLQPEDFATYFCQHYNSYPFTFGQGTKVEIKGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 171 - protein, 235 aa, synthetic construct
EVQLVESGGGLVQPGGSLRLSCAVSGFSLTTYGVDWVRQAPGKGLEWVGIIWGDGTTHYHSALISRFTISKDNSKNTVYLQMNSLRAEDTAVYYCAKPGGASWGQGTLVTVSSGGGGSGGGGSGGGGSDIQMTQSPSSLSASVGDRVTITCKASQNVGTNVAWYQQKPGKSPKALFYSASYRNAGVPSRFSGSGSGTDFTLTISSLQPEDFATYFCQHYNSYPFTFGQGTKVEIK

SEQ ID NO: 172 - DNA, 16 bp, synthetic construct
  source 1..16: mol_type = other DNA
agtactatagcatctg

SEQ ID NO: 173 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 446 aa, synthetic construct
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

(19) United States
(12) Patent Application Publication
(10) Pub. No.: US 2026/0274997 A1
(43) Pub. Date: Sep. 17, 2026

(54) TRANSFERRIN RECEPTOR BINDING MOLECULE CONJUGATES FOR DELIVERY OF OLIGONUCLEOTIDES TO CELLS

(71) Applicant: Denali Therapeutics Inc., South San Francisco, CA (US)

(72) Inventors: Hai L. TRAN, South San Francisco, CA (US); Robert C. WELLS, Foster City, CA (US); Sarah L. DEVOS, San Francisco, CA (US); Mark S. DENNIS, South San Francisco, CA (US)

(21) Appl. No.: 18/875,753

(22) PCT Filed: Jun. 30, 2023
(86) PCT No.: PCT/US2023/069481

§ 371 Date: Dec. 17, 2024

Related U.S. Application Data
(60) Provisional application No. 63/357,959, filed on Jul. 1, 2022.

(51) Int. Cl. C07K 19/00; A61K 39/00; A61K 47/64; A61K 47/68; C07K 16/28

(52) U.S. Cl. CPC C07K 19/00; A61K 47/643; A61K 47/6807; A61K 47/6849; C07K 16/2881; A61K 2039/505; C07K 2317/622

(57) ABSTRACT

Provided herein are conjugates comprising: an anti-TfR antibody antigen binding domain that binds to a transferrin receptor and an oligonucleotide(s). The conjugates can be used to deliver an oligonucleotide to a cell that expresses the transferrin receptor. Delivering the oligonucleotide to a cell can be used to modulate expression of a target gene or sequence in the cell.

### CROSS-REFERENCE TO RELATED APPLICATIONS

This application claims the benefit of U.S. Provisional Application No. 63/357,959, filed Jul. 1, 2022, which is incorporated herein by reference

### SEQUENCE LISTING

The Sequence Listing written in file DNL-041-01-WO_SeqListing.txt is 177 kilobytes in size, was created Jun. 30, 2023, and is hereby incorporated by reference.

### FIELD

The subject matter disclosed herein is directed to TfR binder-oligonucleotide conjugates that binds to a transferrin receptor on a target cell and modulate the expression of a target gene or sequence in that cell, as well as methods of use thereof.

### BACKGROUND

In vivo delivery of nucleic acid-based molecules, such as antisense oligonucleotides or RNAi agents, often requires specific targeting to reach certain tissues or cell types. In particular, delivery to non-hepatic tissues remains an obstacle and has limited the use of such therapies. Delivery of oligonucleotides to the central nervous system (CNS) poses a distinct problem due to the blood brain barrier (BBB). One means to deliver oligonucleotides into the CNS is by intrathecal delivery. However, intrathecal delivery is invasive, has a higher risk of side-effects, and often leads to uneven distribution.

The transferrin receptor can be targeted for the delivery of cancer diagnostics and therapeutics. This type II transmembrane glycoprotein is responsible for cellular iron transport and is found at low levels on the surface of many normal cell types.

What is needed is a therapeutic modality that can target the transferrin receptor for delivery of a cargo to the cell via the transferrin receptor.

### SUMMARY

Described are TfR binder-oligonucleotide conjugates for delivery of the oligonucleotide to the CNS or a cell expressing the transferrin receptor (TfR) comprising: the oligonucleotide linked to an anti-TfR antibody antigen binding domain. The anti-TfR antibody antigen binding domain can be, but is not limited to, an antibody, a single chain antibody, a Fab, a F(ab′)₂, a single chain Fab (scFab), a Fv fragment, a single chain variable fragment (scFv), a bivalent scFv, a heavy chain only antibody variable domain (nanobody, e.g., a VHH or a vNAR), or a nanobody. In some embodiments, the anti-TfR antibody antigen binding domain comprises or consists of a scFv. In some embodiments, the anti-TfR antibody antigen binding domain comprises or consists of a Fab. In some embodiments, the anti-TfR antibody antigen binding domain comprises or consists of a scFab. The anti-TfR antibody antigen binding domain can be from any known antibody that specifically binds TfR. In some embodiments, the anti-TfR antibody antigen binding domain comprises a VH domain comprising CDRs having the sequences of SEQ ID NOs: 12-14 and a VL domain comprising CDRs having the sequences of SEQ ID NOs: 15-17. In some embodiments, the anti-TfR antibody antigen binding domain comprises a VH domain comprising CDRs having the sequences of SEQ ID NOs: 21-23 and a VL domain comprising CDRs having the sequences of SEQ ID NOs: 24-26. In some embodiments, the anti-TfR antibody antigen binding domain comprises a VH domain comprising CDRs having the sequences of SEQ ID NOs: 114-116 and a VL domain comprising CDRs having the sequences of SEQ ID NOs: 117-119. In some embodiments, the anti-TfR antibody antigen binding domain comprises a VH domain comprising CDRs having the sequences of SEQ ID NOs: 126-128 and a VL domain comprising CDRs having the sequences of SEQ ID NOs: 129-131. In some embodiments, the anti-TfR antibody antigen binding domain comprises a VH domain comprising CDRs having the sequences of SEQ ID NOs: 134-136 and a VL domain comprising CDRs having the sequences of SEQ ID NOs: 137-139. In some embodiments, the anti-TfR antibody antigen binding domain comprises a VH domain comprising CDRs having the sequences of SEQ ID NOs: 154-156 and a VL domain comprising CDRs having the sequences of SEQ ID NOs: 157-159. In some embodiments, the anti-TfR antibody antigen binding domain comprises a VH domain comprising CDRs having the sequences of SEQ ID NOs: 161-163 and a VL domain comprising CDRs having the sequences of SEQ ID NOs: 164-166. In some embodiments the anti-TfR antibody antigen binding domain specifically binds human TfR. In some embodiments, the TfR-binding region binds to an apical domain of TfR. The oligonucleotide can be linked to the anti-TfR antibody antigen binding domain directly or indirectly. For indirect attachments, the oligonucleotide can be linked to the anti-TfR antibody antigen binding domain via a chemical linker and/or a peptide. The peptide can be, but is not limited to, a Fc polypeptide, and Fc dimer, or albumin. The oligonucleotide can be, but is not limited to, an antisense oligonucleotide (ASO) or an RNA interference oligonucleotide. The anti-TfR antibody antigen binding domain can have a substitution or modification that facilitates conjugation of the oligonucleotide. The peptide (e.g., the Fc polypeptide, Fc dimer, or albumin), if present, can have a substitution or modification that facilitates conjugation of the oligonucleotide.

In some embodiments, the TfR binder-oligonucleotide conjugate comprises the oligonucleotide linked to an anti-TfR Fab or scFab. In some embodiments, the TfR binder-oligonucleotide conjugate comprises the oligonucleotide linked to monomeric anti-TfR Fab or scFab (mono-Fab). Mono-Fab indicates the TfR binder-oligonucleotide conjugate comprises a single Fab or scFab (i.e., the TfR binder-oligonucleotide conjugate does not contain a second antibody antigen binding domain). In some embodiments, the mono-Fab is linked to a Fc polypeptide or a Fc dimer. In some embodiments, the mono-Fab is linked to a Fc polypeptide or a Fc dimer, wherein the oligonucleotide is linked to the Fc polypeptide, Fc dimer. The Fc polypeptide, Fc dimer, Fab, or scFab can have a substitution or modification that facilitates conjugation of the oligonucleotide.

In some embodiments, the TfR binder-oligonucleotide conjugate comprises or consists of:

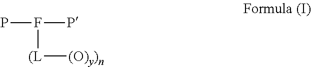

wherein
P comprises an anti-TfR antibody antigen binding domain,
F is optionally present or absent, and if present comprises a peptide, a Fc polypeptide, a Fc dimer, or albumin;
L is optionally present or absent, and if present is a linking group;
P′ is optionally present or absent, and if present comprises an anti-TfR antibody antigen binding domain, a non-binding Fab, a non-binding variable region (NBVR), or a antibody binding domain that does not specifically bind transferrin;
O is an oligonucleotide;
y is an integer greater than or equal to 1 (e.g., 1, 2, 3, or 4); and
n is an integer greater than or equal to 1 (e.g., 1, 2, 3, 4, 5, 6, 7 or 8).
   P-F-P′ can be referred to as a TfR binder. In some embodiments, P-F-P′ comprises an anti-TfR antibody. In some embodiments, P-F comprises an monovalent anti-TfR antibody. If P′ is present and F comprises a Fc dimer, then P, or a heavy chain component of P, can form a single polypeptide chain with one Fc polypeptide of the Fc dimer and P′, or a heavy chain component of P′, can form a single polypeptide chain with the other Fc polypeptide of the Fc dimer. If n is greater than or equal to 2, then y is independently an greater than or equal to 1 (e.g., 1, 2, 3, or 4) for each (L-(O)_{y}). In some embodiments, the oligonucleotide comprises an ASO.

In some embodiments, P is an anti-TfR Fab or scFab, Fis Fc dimer, and P′ is absent. In some embodiments, the oligonucleotide comprises an ASO.

In some embodiments, P is an anti-TfR Fab or scFab, F is Fc dimer, and P′ is a non-binding Fab or NBVR. In some embodiments, the oligonucleotide comprises an ASO.

In some embodiments, P is an anti-TfR scFv, VHH, or nanobody, F is Fc dimer, and P′ absent. In some embodiments, the oligonucleotide comprises an ASO.

In some embodiments, P is an anti-TfR scFv, VHH, or nanobody, F is Fc dimer, and P′ is a non-binding Fab or NBVR. In some embodiments, the oligonucleotide comprises an ASO.

In some embodiments, P is an anti-TfR scFv, VHH, or nanobody, F is an albumin, and P′ absent. In some embodiments, the oligonucleotide comprises an ASO.

In some embodiments, the TfR binder-oligonucleotide conjugate comprises:
a protein comprising:
   an antibody Fc constant domain dimer,
   a first Fab that specifically binds to a transferrin receptor (TfR), and
   a modification for covalent conjugation; and,
an oligonucleotide conjugated at the site of modification.
   The antibody Fc constant domain dimer comprises a first Fc polypeptide and a second Fc polypeptide. In some embodiments, the first Fab comprises a VH domain comprising CDRs having the sequences of SEQ ID NOs: 12-14 and a VL domain comprising CDRs having the sequences of SEQ ID NOs: 15-17. In some embodiments, the first Fab comprises a VH domain comprising CDRs having the sequences of SEQ ID NOs: 21-23 and a VL domain comprising CDRs having the sequences of SEQ ID NOs: 24-26. In some embodiments, the first Fab comprises a VH domain comprising CDRs having the sequences of SEQ ID NOs: 114-116 and a VL domain comprising CDRs having the sequences of SEQ ID NOs: 117-119. In some embodiments, the first Fab comprises a VH domain comprising CDRs having the sequences of SEQ ID NOs: 126-128 and a VL domain comprising CDRs having the sequences of SEQ ID NOs: 129-131. In some embodiments, the first Fab comprises a VH domain comprising CDRs having the sequences of SEQ ID NOs: 134-139 and a VL domain comprising CDRs having the sequences of SEQ ID NOs: 137-139. In some embodiments, the first Fab comprises a VH domain comprising CDRs having the sequences of SEQ ID NOs: 154-156 and a VL domain comprising CDRs having the sequences of SEQ ID NOs: 157-159. In some embodiments, the first Fab comprises a VH domain comprising CDRs having the sequences of SEQ ID NOS: 161-163 and a VL domain comprising CDRs having the sequences of SEQ ID NOs: 164-166. The first Fab can be linked to the first Fc polypeptide or the second Fc polypeptide to form a Fab-Fc fusion. In some embodiments, the TfR binder-oligonucleotide conjugate further comprises a second Fab. The second Fab can be, but is not limited to, a Fab that specifically binds to a TfR, a non-binding Fab, or a non-binding variable region (NBVR). The second Fab can be linked to the first Fc polypeptide or the second Fc polypeptide to form a Fab-Fc fusion In some embodiments, the TfR binder-oligonucleotide conjugate comprises a second Fab, wherein the first Fab is linked to the first Fc polypeptide and the second Fab is linked to the second Fc polypeptide. In some embodiments, the TfR binder-oligonucleotide conjugate comprises a second Fab, wherein the first Fab is linked to the second Fc polypeptide and the second Fab is linked to the first Fc polypeptide. In some embodiments, the oligonucleotide is conjugated to the antibody via a linker “L.”

In certain embodiments, the TfR binder-oligonucleotide conjugate comprises an antibody-oligonucleotide conjugate comprising:
an antibody that binds to a transferrin receptor (TfR), wherein the antibody comprises heavy chain CDRs of SEQ ID NOs: 12-14, 21-23, 114-116, 126-128, 134-136, 154-156, or 161-163 and light chain CDRs of SEQ ID NOs: 15-17, 24-26, 117-119, 129-131, 137-139, 157-159, or 164-166; and
an oligonucleotide conjugated to a cysteine modification on the constant domain of the antibody.
   In some embodiments, the oligonucleotide is conjugated to the antibody via a linker “L.”

In certain embodiments, the TfR binder-oligonucleotide conjugate comprises an antibody-oligonucleotide conjugate comprising:
an antibody that binds to a transferrin receptor (TfR), wherein the antibody comprises heavy chain CDRs of SEQ ID NOs: 12-14, 21-23, 114-116, 126-128, 134-136, 154-156, or 161-163 and light chain CDRs of SEQ ID NOs: 15-17, 24-26, 117-119, 129-131, 137-139, 157-159, or 164-166; and
an oligonucleotide conjugated to a cysteine modification on the constant domain of the antibody.

In certain embodiments, the TfR binder-oligonucleotide conjugates described herein have the structure:

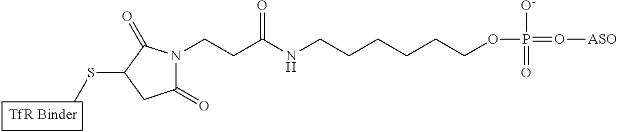

In certain embodiments, the subject matter described herein is directed to a method of modulating the expression of a target gene in the muscle cell or CNS cell of a patient, comprising administering to the patient a conjugate as described herein or a pharmaceutical composition comprising the conjugate.

These and other embodiments are fully described herein.

### BRIEF DESCRIPTION OF THE FIGURES

FIG. 1 illustrates huIgG, intact drug, % intact drug, and total ASO in CNS, i.e., cortex, spinal cord, for TfR mono-Fab conjugate 24 hours post single dose.

FIG. 2A illustrates huIgG and intact drug in CNS, i.e., cortex, spinal cord, for TfR mono-Fab conjugate 72 hours post final dose in multi-dose study.

FIG. 2B illustrates total ASO and Malat1 knockdown in CNS, i.e., cortex, spinal cord, for TfR mono-Fab conjugate 72 hours post final dose in multi-dose study.

FIG. 3 illustrates huIgG, intact drug, % intact drug, and total ASO in the periphery for TfR mono-Fab conjugate 24 hours post single dose.

FIG. 4 illustrates huIgG, intact drug, and total ASO for TfR mono-Fab conjugate 72 hours post final dose in multi-dose study.

FIG. 5 illustrates Malat1 for TfR mono-Fab conjugate 72 hours post final dose in multi-dose study.

FIG. 6 illustrates Malat1 knockdown in CNS and periphery for anti-TfR bivalent antibody conjugate.

FIG. 7 illustrates plasma clearance of TfR-mono Fab:ASO conjugates.

FIG. 8 illustrates huIgG concentrations in brain, spinal cord, and peripheral tissues of TfR-mono Fab:ASO conjugates.

FIG. 9 illustrates ASO concentrations in tissues in brain, spinal cord, and peripheral tissues of TfR-mono Fab:ASO conjugates. Unconjugated ASO is the first bar for each tissue (not visible for Brain and SC). TfR-mono Fab is the middle bar for each tissue. TfR-mono Fab 2 is the third bar for each tissue.

FIG. 10 illustrates ASO concentration in brain at 72 hours for Tfr Albumin:ASO conjugate.

FIG. 11 illustrates ASO concentration in kidney and liver at 72 hours for Tfr Albumin:ASO conjugate.

FIG. 12 illustrates plasma clearance of Tfr Albumin:ASO conjugate.

FIG. 13 illustrates exemplary TfR binder-oligonucleotide conjugates having (a) an anti-TfR Fab/non-binding Fab antibody (upper left), (b) an anti-TfR mono-Fab antibody (upper right), and (c) an anti-TfR scFv-albumin (lower). (a), (b), and (c) are shown with an ASO attached. (c) is shown with an optional 6×His tag.

### DETAILED DESCRIPTION

### I. Introduction

Oligonucleotide therapies for disorders caused by genetic abnormalities or increased protein accumulation are becoming an increasingly popular approach to modulate gene expression to treat the disorders. Delivery of the oligonucleotide to the cell remains a challenge. Disclosed herein are TfR binder-oligonucleotide conjugates that exploit the transferrin receptor to deliver the oligonucleotide to the target cells.

In certain embodiments, the described TfR binder-oligonucleotide conjugates are capable of crossing the blood brain barrier (BBB). Generally, the BBB represents a challenge to the delivery of systemically administered oligonucleotides to the relevant sites of action within the CNS. Intrathecal (IT) delivery, in which drugs are administered directly into the cerebrospinal fluid (CSF) space, enables the bypass of the BBB. However, one limitation of this approach is that delivery of these oligonucleotide therapies directly to the CSF via the IT approach does not achieve effective distribution in the CNS.

In certain embodiments, the TfR binder-oligonucleotide conjugates are capable of delivering the conjugated oligonucleotide to the CNS or a cell expressing a transferrin receptor. The cell can be, but is not limited to, a muscle cell or a cancer cell. The muscle cell can be, but is not limited to, a skeletal muscle cell or a cardiac cell.

Described herein are TfR binder-oligonucleotide conjugates and methods of use thereof. In some embodiments, the TfR binder comprises a monovalent antibody (mono-Fab; i.e., an antibody having a single anti-TfR antibody antigen binding domain, e.g., a single Fab arm or scFv). In some embodiments, the TfR binder comprises a bispecific divalent antibody (i.e., an antibody having a single anti-TfR antibody antigen binding domain and a single non-binding Fab or NBVR). In some embodiments, the TfR binder comprises an anti-TfR scFab, svFc, VHH, vNAR or nanobody linked to an albumin. In some embodiments, the TfR binder comprises bivalent anti-TfR antibody (e.g., anti-TfR (Fab) 2). In some embodiments, a TfR binder-oligonucleotide conjugate comprises an oligonucleotide covalently linked to and anti-TfR antibody.

In certain embodiments, the oligonucleotide is conjugated at a cysteine modification on the constant domain of the antibody, a Fc polypeptide, or a Fc dimer.

An anti-TfR antibody antigen binding domain for use in forming a TfR binder can be derived from an antibody known to have affinity to the transferrin receptor. Derived from indicates the anti-TfR antibody antigen binding domain comprises the antibody, and antigen binding fragment of the antibody, or an antigen binding region having the CDR sequences of the antibody. Examples of antibodies or protein molecules that can be used to conjugate an oligonucleotide include those described in WO2014/033074, WO2016/081640 and WO2020/132584, each of which is incorporated herein by reference in its entirety.

The oligonucleotide can also be referred to as a cargo that is delivered to the target cell by the TfR binder-oligonucleotide conjugate. The oligonucleotide can be, but is not limited to, an antisense oligonucleotide (“ASO”) or an RNAi agent (e.g., a siRNA or shRNA).

In further embodiments, provided herein are treatment methods and methods of using a conjugate as described herein to target an oligonucleotide (e.g., ASO or an RNAi agent) to transferrin receptor-expressing cells, e.g., to deliver the oligonucleotide to that cell.

### II. Definitions

As used herein, the singular forms “a,” “an,” and “the” include plural referents unless the content clearly dictates otherwise.

As used herein, the terms “about” and “approximately,” when used to modify an amount specified in a numeric value or range, indicate that the numeric value as well as reasonable deviations from the value known to the skilled person in the art, for example±20%, ±10%, or ±5%, are within the intended meaning of the recited value.

The term “non-targeting Fab fragment” refers to a Fab fragment that does not specifically bind to an antigen via its heavy or light chain variable domains or does not specifically bind to an antigen expressed in a given mammal, such as a primates, e.g., human and non-human primates, or rodents, e.g., mouse, or in a particular tissue within such a mammal via its heavy or light chain variable domains.

A “transferrin receptor” or “TfR” refers to transferrin receptor protein 1. The human transferrin receptor 1 polypeptide sequence is set forth in SEQ ID NO: 2. Transferrin receptor protein 1 sequences from other species are also known (e.g., chimpanzee, accession number XP_003310238.1; rhesus monkey, NP_001244232.1; dog, NP_001003111.1; cattle, NP_001193506.1; mouse, NP_035768.1; rat, NP_073203.1; and chicken, NP_990587.1). The term “transferrin receptor” also encompasses allelic variants of the exemplary reference sequences, e.g., human sequences, that are encoded by a gene at a transferrin receptor protein 1 chromosomal locus. Full length transferrin receptor protein includes a short N-terminal intracellular region, a transmembrane region, and a large extracellular domain. The extracellular domain is characterized by three domains: a protease-like domain, a helical domain, and an apical domain. The apical domain comprises residues 189-383 of human TfR. The apical domain sequence of human transferrin receptor 1 is set forth in SEQ ID NO: 3.

The term “constant domain” refers to a light chain constant region domain polypeptide (CL) and CH1, CH2 and CH3 domain polypeptides from the heavy chain.

The terms “CH1 domain”, “CH3 domain” and “CH2 domain” refer to immunoglobulin constant region domain polypeptides. In the context of IgG antibodies, a CH3 domain polypeptide refers to the segment of amino acids from about position 341 to about position 447 as numbered according to the EU numbering scheme, a CH2 domain polypeptide refers to the segment of amino acids from about position 231 to about position 340 as numbered according to the EU numbering scheme, and a CH1 domain polypeptide refers to the segment of amino acids from about position 118 to about position 215 according to the EU numbering scheme. CH1, CH2 and CH3 domain polypeptides may also be numbered by the IMGT (ImMunoGeneTics) numbering scheme in which the CH1 domain numbering is 1-98, the CH2 domain numbering is 1-110 and the CH3 domain numbering is 1-107, according to the IMGT Scientific chart numbering (IMGT website). CH2 and CH3 domains are part of the Fc polypeptide of an immunoglobulin. In the context of IgG antibodies, an Fc polypeptide refers to the segment of amino acids from about position 231 to about position 447 as numbered according to the EU numbering scheme.

The term “variable domain” refers to a light chain variable region domain polypeptide (VL) and a heavy chain variable region domain polypeptide (VH). The VL contains three complementarity-determining regions (CDR) regions, CDR-L1, CDR-L2, CDR-L3, and the VH contains three CDR regions, CDR-H1, CDR-H2, and CDR-H3. The CDR regions together form the antibody-binding site that binds to an antigen.

The term “Fc polypeptide” refers to the C-terminal region of a naturally occurring immunoglobulin heavy chain polypeptide that is characterized by an Ig fold as a structural domain. An Fc polypeptide typically contains constant region sequences including at least the CH2 domain and/or the CH3 domain and may contain at least part of the hinge region. Illustrative hinge region sequences, or portions thereof, are set forth in SEQ ID NOs: 4-6.

An “Fc polypeptide dimer” refers to a dimer of two Fe polypeptides. In some embodiments, an Fc polypeptide dimer is capable of binding an Fc receptor (e.g., FcγR). In an Fc polypeptide dimer, the two Fc polypeptides dimerize by the interaction between the two CH3 antibody constant domains. In some embodiments, the two Fc polypeptides may also dimerize via one or more disulfide bonds that form between the hinge domains of the two dimerizing Fc domain monomers. An Fc polypeptide dimer can be a heterodimer or a homodimer. An Fc polypeptide dimer may comprise two wild-type Fc polypeptides, a wild-type Fc polypeptide and a modified Fc polypeptide, or two modified Fc polypeptides. For an Fc polypeptide dimer comprising two modified Fc polypeptide, the two modified Fc polypeptides may be the same or different.

An antibody antigen binding domain comprises the antigen binding domain of an immunoglobulin or a peptide having a structure similar to the antigen binding domain of an immunoglobulin. The immunoglobulin can be, but is not limited to, an IgG, IgM, IgE, IgA, IgD, or a heavy chain antibody. An antibody antigen binding domain can be, but is not limited to, a Fab, a scFab, a Fv fragment, a scFv, or a heavy chain only antibody variable domain (nanobody, e.g., a VHH or a vNAR).

The term “CL domain” refers to the immunoglobulin constant domain of the light chain. In the context of IgG antibodies, a kappa CL domain polypeptide refers to the segment of amino acids from about position 108 to about position 214 as numbered according to the EU numbering scheme. Alternatively, the kappa and lambda CL domains may be numbered by the IMGT (ImMunoGeneTics) numbering scheme in which the kappa CL domain numbering is 1-107, and the lambda CL domain numbering is 1-106, according to the IMGT Scientific chart numbering (IMGT website).

The term “Fab” or “Fab fragment” refers to a monovalent fragment consisting of a VL, VH, CL and CH1 domain. The term “Fab” refers to a monovalent antigen-binding fragment consisting of a light chain variable region (VL) and a light chain constant region (CL) (together the antibody light chain), and a heavy chain variable region (VH) and a heavy chain CH1 constant region (together an antibody Fd fragment). A Fab or Fab fragment may or may not contain all or part of an antibody hinge region.

The term “single-chain Fab” or “scFab” refers to an antigen-binding fragment consisting of a Fab wherein the Fd fragment and the light chain linked together via a peptide linker. The linker can connect the N-terminus of the Fd fragment with the C-terminus of the light chain or the N-terminus of the light chain with the C-terminus of the Fd fragment.

The term “Fv fragment” refers to an antigen-binding fragment consisting of a V_{H} and a V_{L} that together form a binding site for an antigen.

The term “single-chain variable fragment” or “scFv” refers to an antigen-binding fragment consisting of a heavy chain variable region and a light chain variable region linked together via a peptide linker. The linker can connect the N-terminus of the V_{H} with the C-terminus of the V_{L} or the N-terminus of the VL with the C-terminus of the V_{H}. An scFv lacks constant regions. Modified scFv and methods of modifying a scFv to bind to a target protein are described in WO 2022/258841 (which is incorporated herein by reference).

The term “nanobody” refers to an antibody fragment consisting of a single monomeric variable antibody domain. Nanobodies derived from camelid heavy chain antibodies can be referred to as “VHH” fragments. Nanobodies derived from cartilaginous fish heavy chain antibodies can be referred to as “vNARs.” Modified VHH fragments and methods of modifying a VHH fragment to bind to a target protein, including TfR are described in WO 2020/056327, WO 2022/103769, and WO 2023/023166 (each of which is incorporated herein by reference).

The term “non-targeting Fab fragment” or “NTF” refers to a Fab fragment that does not specifically bind to a naturally occurring human antigen via its heavy or light chain variable domains or does not specifically bind to an naturally occurring antigen expressed in a given mammal, such as a primates, e.g., human and non-human primates, or rodents, e.g., mouse, or in a particular tissue within such a mammal via its heavy or light chain variable domains. In certain embodiments, a Fab for use in a Fab-Fc fusion or Fab-Fc dimer fusion as described herein does not specifically bind to transferrin via its heavy or light chain variable domains. Non-limiting examples of non-targeting Fab fragments include (a) RSV (palivizumab) Fab fragments, which are non-targeting in mice and non-human primates, and (b) Fab fragments to dinitrophenyl hapten (DNP) (See Leahy, PNAS 3661-3665, 1988).

The terms “wild-type,” “native,” and “naturally occurring” with respect to a CH3 or CH2 domain refers to a domain that has a sequence that occurs in nature.

As used herein, the term “mutant” with respect to a mutant polypeptide or mutant polynucleotide is used interchangeably with “variant.” A variant with respect to a given wild-type CH3 or CH2 domain reference sequence can include naturally occurring allelic variants. A “non-naturally” occurring CH3 or CH2 domain refers to a variant or mutant domain that is not present in a cell in nature and that is produced by genetic modification, e.g., using genetic engineering technology or mutagenesis techniques, of a native CH3 domain or CH2 domain polynucleotide or polypeptide. A “variant” includes any domain comprising at least one amino acid mutation with respect to wild-type. Mutations may include substitutions, insertions, and deletions.

The term “modified site” refers to a particular position within a polypeptide that comprises a mutation or variant, with respect to a corresponding wild-type polypeptide (e.g., a wild-type CL, CH1, CH2 or CH3 domain). In certain embodiments, the mutation or variant is non-naturally occurring. A modified site may include, e.g., an insertion or a substitution. The term “substitution” refers to an alteration that replaces an amino acid with another amino acid. For example, a “cysteine substitution” or “cysteine modification” refers to the replacement of an amino acid with a cysteine. A modification can be indicated using the notation XnumberY, wherein X represents the amino acid in a parent polypeptide at the position indicated by the number, and Y represents the substitute amino acid to which replaces amino acid X. For example, S239C indicates a serine at position 239 is replace by a Cysteine.

The term “amino acid” refers to naturally occurring and synthetic amino acids, as well as amino acid analogs and amino acid mimetics that function in a manner similar to the naturally occurring amino acids.

Naturally occurring amino acids are those encoded by the genetic code, as well as those amino acids that are later modified, e.g., hydroxyproline, γ-carboxyglutamate and O-phosphoserine. “Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that function in a manner similar to a naturally occurring amino acid.

Naturally occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Amino acids may be referred to herein by either their commonly known three letter symbols or by the one-letter symbols recommended by the IUPAC-IUB Biochemical Nomenclature Commission.

The terms “polypeptide” and “peptide” are used interchangeably to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” refers to either a single chain polypeptide or a dimer (i.e., two) or multimer (i.e., three or more) of single chain polypeptides. The single chain polypeptides of a dimer or multimer may be joined by a covalent bond, e.g., a disulfide bond, or non-covalent interactions.

The term “conservative substitution,” “conservative mutation,” or “conservatively modified variant” refers to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Trp (Tryptophan or W), and (Histidine or H); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged sub-group” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free-OH can be maintained; and Gln for Asn or vice versa, such that a free —NH₂ can be maintained. In some embodiments, hydrophobic amino acids are substituted for naturally occurring hydrophobic amino acid, e.g., in the active site, to preserve hydrophobicity.

The terms “identical” or percent “identity,” in the context of two or more polypeptide sequences, refer to two or more sequences or subsequences that are the same or have a specified percentage of amino acid residues, e.g., at least 60% identity, at least 65%, at least 70%, at least 75%, at least 80%, at least 85%, at least 90%, or at least 95% or greater, that are identical over a specified region when compared and aligned for maximum correspondence over a comparison window, or designated region as measured using one a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “corresponding to,” “determined with reference to,” or “numbered with reference to” when used in the context of the identification of a given amino acid residue in a polypeptide or protein sequence, refers to the position of the residue of a specified reference sequence when the given amino acid sequence is maximally aligned and compared to the reference sequence. Thus, for example, an amino acid residue in a polypeptide “corresponds to” an amino acid in the region of SEQ ID NO: 1 from amino acids 114-220 when the residue aligns with the amino acid in SEQ ID NO: 1 when optimally aligned to SEQ ID NO: 1. The polypeptide that is aligned to the reference sequence need not be the same length as the reference sequence.

“Binding affinity” refers to the strength of the non-covalent interaction between two molecules, e.g., a single binding site on a polypeptide/protein and a target, e.g., transferrin receptor, to which it binds. Thus, for example, the term may refer to 1:1 interactions between a polypeptide/protein and its target, unless otherwise indicated or clear from context. Binding affinity may be quantified by measuring an equilibrium dissociation constant (K_{D}), which refers to the dissociation rate constant (k_{d}, time⁻¹) divided by the association rate constant (ka, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet® platform). As used herein, “binding affinity” includes not only formal binding affinities, such as those reflecting 1:1 interactions between a polypeptide/protein and its target, but also apparent affinities for which K_{D}'s are calculated that may reflect avid binding.

The phrase “specifically binds” or “selectively binds” to a target, e.g., transferrin receptor, refers to a binding reaction whereby the protein binds to the target with greater affinity, greater avidity, and/or greater duration than it binds to a structurally different target, e.g., a target not in the transferrin receptor family. In typical embodiments, the protein has at least 5-fold, 10-fold, 100-fold, 1000-fold, 10,000-fold or greater affinity for a transferrin receptor compared to an unrelated target when assayed under the same affinity assay conditions. In some embodiments, a protein may bind exclusively to a human transferrin receptor.

The terms “nucleic acid” and “polynucleotide” refer to deoxyribonucleotides or ribonucleotides and polymers thereof in either single- or double-stranded form, composed of monomers (nucleotides) containing a sugar moiety, phosphate and a nucleobase. Unless specifically limited, the term encompasses both modified and unmodified nucleic acids.

The term “nucleobase” refers to nitrogen-containing compounds that can be linked to a sugar moiety to form nucleosides, which in turn are components of nucleotides. The ability of nucleobases to form base pairs and to stack one upon another leads directly to long-chain helical structures such as ribonucleic acid (RNA) and deoxyribonucleic acid (DNA). Nucleobases may be naturally occurring (i.e., adenine (A), cytosine (C), guanine (G), thymine (T), and uracil (U)) or modified.

The term “nucleoside” refers to a compound comprising a nucleobase and sugar moiety (e.g., deoxyribose or ribose, or a modified variant thereof). The term nucleoside includes both modified and unmodified nucleosides.

The term “nucleotide” refers to a compound comprising a nucleobase, a sugar moiety, and one or more phosphate groups. The term nucleotide includes both modified and unmodified nucleotides.

The term “internucleoside linkage” means the covalent linkages between two nucleosides in an oligonucleotide. Nucleosides may be linked via natural (i.e., a phophodiester (PO) linkage) or modified linkages.

The terms “chemical modification”, “modification” or “modified” may refer to a chemical change in a compound when compared to its naturally occurring counterpart. For example, a nucleobase, a sugar moiety or an internucleoside linkage may be chemically modified. An amino acid in a protein or polypeptide may be modified. The modification may be a modification to the existing amino acid or a substitution of one amino acid for another. An example a modification of one amino acid for another, includes, but it not limited to, a cysteine modification, wherein a naturally occurring amino acid at a position is replaced by a cysteine (i.e., a cysteine modification).

The terms “nucleotide sequence” and “nucleic acid sequence” and “nucleic acid strand” refer to a sequence of bases (purines and/or pyrimidines, or synthetic derivatives thereof) in a polymer of DNA or RNA, which can be single-stranded or double-stranded, optionally containing synthetic, non-natural or altered nucleotides capable of incorporation into DNA or RNA polymers, and/or backbone modifications (e.g., a modified oligomer). The terms “oligo”, “oligonucleotide” and “oligomer” may be used interchangeably and refer to such sequences of purines and/or pyrimidines. For example, the oligonucleotide may comprise chemically modified or unmodified nucleic acid molecules (RNA or DNA) having a length of less than about, e.g., about 200 nucleotides (for example, less than about 100 or 50 nucleotides). The oligonucleotide can, e.g., be single stranded DNA or RNA (e.g., an ASO); double stranded DNA or RNA (e.g., small interfering RNA (siRNA)), including double stranded DNA or RNA having a hairpin loop; or DNA/RNA hybrids. In one embodiment, the oligonucleotide has a length ranging from about 5 to about 60 nucleotides, or about 10 to about 50 nucleotides. In another embodiment, the oligonucleotide has a length ranging from about 5 to about 30 nucleotides or from about 15 to about 30 nucleotides. In yet another embodiment, the oligonucleotide has a length ranging from about 18 to about 24 nucleotides.

The terms “modified oligos”, “modified oligonucleotides” or “modified oligomers” may be similarly used interchangeably, and refer to such sequences that contain synthetic, non-natural or altered bases, sugars and/or backbone modifications.

The oligonucleotides described herein may be synthesized using standard solid or solution phase synthesis techniques that are known in the art. In certain embodiments, the oligonucleotides are synthesized using solid-phase phosphoramidite chemistry (U.S. Pat. No. 6,773,885) with automated synthesizers. Chemical synthesis of nucleic acids allows for the production of various forms of the nucleic acids with modified linkages, chimeric compositions, and nonstandard bases or modifying groups attached in chosen places through the nucleic acid's entire length.

The term “complementary” as used herein refers to the broad concept of complementary base pairing between two nucleic acids aligned in an antisense position in relation to each other. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, at least about 60%, or at least about 80% of corresponding positions in each of the molecules are occupied by nucleotides which normally base pair with each other (e.g., A:T (A:U for RNA) and G:C nucleotide pairs).

The terms “identical” or percent “identity,” in the context of two or more nucleotide sequences, refer to two or more sequences or subsequences that are the same or have a specified percentage of nucleotides, e.g., at least 60% identity, at least 65%, at least 70%, at least 75%, at least 80%, at least 85%, at least 90%, or at least 95% or greater, that are identical over a specified region when compared and aligned for maximum correspondence over a comparison window, or designated region as measured using one a sequence comparison algorithm or by manual alignment and visual inspection.

For sequence comparison of oligonucleotides (e.g., to determine identity or complementarity), typically one nucleotide sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art.

As used herein, “hybridize” or “hybridization” means the pairing of complementary nucleotide sequences (e.g., an antisense compound and its target nucleic acid; or between antisense and sense strands). As used herein, “specifically hybridizes” means the ability of a reference nucleic acid to hybridize to one nucleic acid molecule with greater affinity than it hybridizes to another.

“Expression” refers to the transcription and/or translation of an endogenous gene, heterologous gene or nucleic acid segment, or a transgene in cells. For example, expression may refer to the transcription and stable accumulation of sense (mRNA) or functional RNA. Expression may also refer to the production of protein.

The term “gene” refers to a nucleic acid (e.g., DNA or RNA) sequence that comprises coding sequences necessary for the production of a polypeptide or precursor.

The phrase “modulating the expression of a target gene or sequence” means a change (e.g., an increase or decrease) in expression of the target gene or sequence (e.g., via degradation of the target or translation inhibition). For example, it includes inhibiting, reducing or decreasing the expression of a target gene or sequence. This also includes a change in alternative splicing, which may result in a change in the absolute or relative amount of a particular splice variant.

The term “halo” is fluoro, chloro, bromo, or iodo. Alkyl, alkoxy, etc. denote both straight and branched groups; but reference to an individual radical such as propyl embraces only the straight chain radical, a branched chain isomer such as isopropyl being specifically referred to.

The term “alkyl”, by itself or as part of another substituent, means, unless otherwise stated, a straight or branched chain hydrocarbon radical, having the number of carbon atoms designated (i.e., C₁₋₆ means one to six carbons). Examples include (C₁-C₆)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and higher homologs and isomers.

The term “alkoxy” refers to an alkyl groups attached to the remainder of the molecule via an oxygen atom (“oxy”).

The term “alkylthio” refers to an alkyl groups attached to the remainder of the molecule via a thio group.

The term “alkoxycarbonyl” refers to a group (alkyl)-O—C(═O)—, wherein the term alkyl has the meaning defined herein.

The term “alkanoyloxy” refers to a group (alkyl)-C(═O)—O—, wherein the term alkyl has the meaning defined herein.

The term “aryloxy” refers to an aryl group attached to the remainder of the molecule via an oxygen atom (Aryl-O—).

The term “heteroaryloxy” refers to a heteroaryl group attached to the remainder of the molecule via an oxygen atom (Heteroaryl-O—).

As used herein, the term “heteroatom” includes oxygen (O), nitrogen (N), sulfur(S) and silicon (Si).

The term “cycloalkyl” refers to a saturated or partially unsaturated (non-aromatic) all carbon ring having 3 to 6 carbon atoms (i.e., (C₃-C₆) carbocycle). Non-limiting examples of cycloalkyls include cyclopropyl, cyclobutyl, cyclopentyl, and cyclohexyl.

The term “aryl” refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed carbon ring systems (e.g., ring systems comprising 2, 3 or 4 rings) having about 9 to 20 carbon atoms in which at least one ring is aromatic and wherein the other rings may be aromatic or not aromatic (i.e., cycloalkyl. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, indanyl, naphthyl, 1, 2, 3, 4-tetrahydronaphthyl, anthracenyl, and the like.

The term “heterocycle” refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from cycloalkyl, aryl, and heterocycle to form the multiple Condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heterocycle) can be at any position of the multiple condensed ring system including a heterocycle, aryl and carbocycle portion of the ring. In one embodiment the term heterocycle includes a 3-12 membered heterocycle. In one embodiment the term heterocycle includes a 3-7 membered heterocycle. In one embodiment the term heterocycle includes a 3-6 membered heterocycle. In one embodiment the term heterocycle includes a 4-6 membered heterocycle. In one embodiment the term heterocycle includes a 3-12 membered monocyclic or bicyclic heterocycle heterocycle comprising 1 to 3 heteroatoms. In one embodiment the term heterocycle includes a 3-6 membered monocyclic heterocycle comprising 1 to 2 heteroatoms. In one embodiment the term heterocycle includes a 4-6 membered monocyclic heterocycle comprising 1 to 2 heteroatoms. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one imidazolidine, pyrazolidine, butyrolactam, valerolactam, imidazolidinone, hydantoin, dioxolane, phthalimide, 1,4-dioxane and

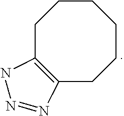 [SMILES: C1CCCc2[nH]nnc2CC1]

In one embodiment the heterocycle can be di-valent, i.e., attached to the remainder of the molecule or the linking group at two positions of the heterocycle (-heterocycle-). In one embodiment, the heterocycle is substituted with one or more (e.g., 1, 2, 3, or 4) substituents independently selected from the group consisting of (C₁-C₆)alkoxy, (C₃-C₆) cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), and carboxy.

As used herein a wavy line “wow” that intersects a bond in a chemical structure indicates the point of attachment of the bond that the wavy bond intersects in the chemical structure to the remainder of a molecule.

The term “subject,” “individual,” and “patient,” as used interchangeably, refer to a mammal, including but not limited to humans, non-human primates, rodents (e.g., rats, mice, and guinea pigs), rabbits, cows, pigs, horses, and other mammalian species. In one embodiment, the patient is a human.

The terms “treatment,” “treating,” and the like are used herein to generally mean obtaining a desired pharmacologic and/or physiologic effect. “Treating” or “treatment” may refer to any indicia of success in the treatment or amelioration of an injury, disease, or condition, including any objective or subjective parameter such as abatement, remission, improvement in patient survival, increase in survival time or rate, diminishing of symptoms or making the injury, disease, or condition more tolerable to the patient, slowing in the rate of degeneration or decline, or improving a patient's physical or mental well-being. Additionally, “treating” or “treatment” may refer to the modulation of the target gene expression such as gene knockdown or gene knockout. For instance, the expression of the target gene or sequence is inhibited or reduced, e.g., by at least about 10%, 20%, 30%, 40%, 50%, 60%, 70%, 80%, 90%, 95%, 99% or 100%, as compared to the expression in a control. The treatment or amelioration of symptoms can be based on objective or subjective parameters. The effect of treatment can be compared to an individual or pool of individuals not receiving the treatment, or to the same patient prior to treatment or at a different time during treatment.

The term “pharmaceutically acceptable excipient” refers to a non-active pharmaceutical ingredient that is biologically or pharmacologically compatible for use in humans or animals, such as but not limited to a buffer, carrier, or preservative.

A “therapeutic amount” or “therapeutically effective amount” of an agent is an amount of the agent that treats, alleviates, abates, or reduces the severity of symptoms of a disease in a subject. A “therapeutic amount” or “therapeutically effective amount” of an agent may improve patient survival, increase survival time or rate, diminish symptoms, make an injury, disease, or condition more tolerable, slow the rate of degeneration or decline, or improve a patient's physical or mental well-being.

The term “administer” refers to a method of delivering agents, compounds, or compositions to the desired site of biological action. These methods include, but are not limited to, topical delivery, parenteral delivery, intravenous delivery, intradermal delivery, intramuscular delivery, intrathecal delivery, colonic delivery, rectal delivery, or intraperitoneal delivery. In one embodiment, the proteins described herein are administered intravenously.

### III. TfR Binder-Oligonucleotide Conjugates

In some embodiments, a TfR binder-oligonucleotide conjugate comprises or consists of:

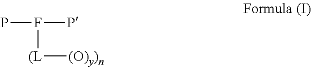

wherein
P comprises an anti-TfR antibody antigen binding domain,
F is optionally present or absent, and if present comprises a peptide, a Fc polypeptide, a Fc dimer, or albumin;
L is optionally present or absent, and if present is a linking group;
P′ is optionally present or absent, and if present comprises an anti-TfR antibody antigen binding domain or a non-binding Fab or non-binding variable region (NBVR);
O is an oligonucleotide;
y is an integer greater than or equal to 1 (e.g., 1, 2, 3, or 4); and
n is an integer greater than or equal to 1 (e.g., 1, 2, 3, 4, 5, 6, 7 or 8).

P-F-P′ (optionally P-F or P if P′ or F and P′ are absent) can be referred to as a TfR binder. In some embodiments, P-F-P′ comprises an anti-TfR antibody. The antibody can be a divalent antibody (having two Fab arm that each specifically bind TfR) or bispecific (having a first Fab arm that specifically binds TfR and a second Fab arm that does not specifically bind TfR). If P′ is present and F comprises a Fc dimer, then P, or a heavy chain component of P, can form a single polypeptide chain with one Fc polypeptide of the Fc dimer and P′, or a heavy chain component of P′, can form a single polypeptide chain with the other Fc polypeptide of the Fc dimer. If n is greater than or equal to 2, then y is independently an greater than or equal to 1 (e.g., 1, 2, 3, or 4) for each (L-(O)_{y}). In some embodiments, the oligonucleotide comprises an ASO. In some embodiments, the TfR binder (P, P′ (if present), and/or F (if present)) comprises at least one substitution or modification that facilitates covalent conjugation of the oligonucleotide O, optional via the linker L.

### A. Monovalent Anti-TfR (Mono-Fab) Antibody-Oligonucleotide Conjugate

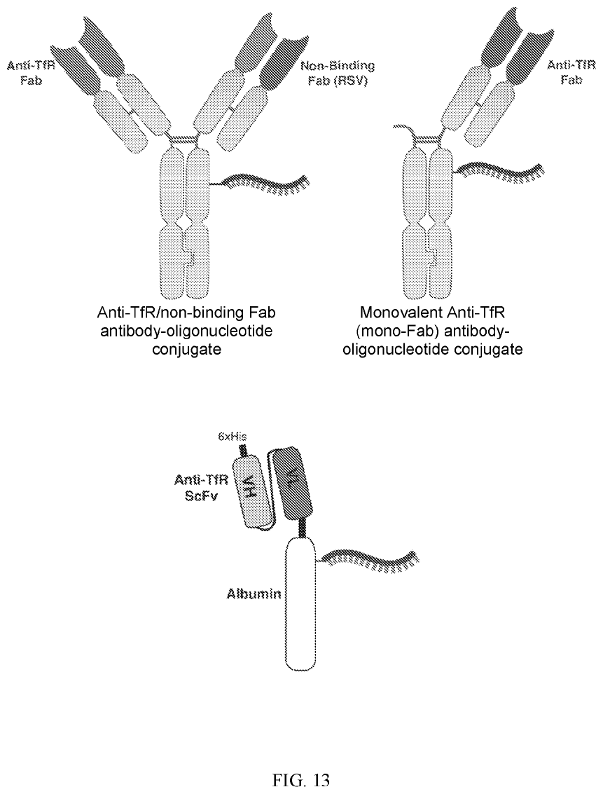

In some embodiments, the TfR binder-oligonucleotide conjugate comprises or consists of:

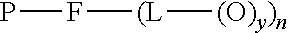

wherein
P comprises an anti-TfR antibody antigen binding domain,
F comprises a Fc polypeptide or Fc dimer;
L is linking group;
O is an oligonucleotide;
y is an integer greater than or equal to 1 (e.g., 1, 2, 3, or 4); and
n is an integer greater than or equal to 1 (e.g., 1, 2, 3, 4, 5, 6, 7 or 8);
   wherein the TfR binder-oligonucleotide conjugate comprises a single anti-TfR antibody antigen binding domain and does not contain any additional antibody antigen binding domain, non-binding Fab, or NBVR (e.g., as shown in FIG. 13). In some embodiments, P comprises an anti-TfR Fab. In some embodiments, P comprises an anti-TfR scFv. In some embodiments, anti-TfR antibody antigen binding domain comprises an anti-TfR VHH, vNAR, or nanobody. The anti-TfR antibody antigen binding domain can be from any anti-TfR antibody known to specifically bind TfR. In some embodiments, F comprises an Fc dimer. In some embodiments, the oligonucleotide comprises an ASO. In some embodiments, the TfR binder (P and/or F) comprises at least one substitution or modification that facilitates covalent conjugation of the oligonucleotide O, optionally via the linker L. The oligonucleotide can be linked to Por F. If the oligonucleotide is linked to F and F is an Fc dimer), it can be linked to the Fc polypeptide linked to P or the Fc polypeptide not linked to P.

### B. Anti-TfR/Non-Binding Fab Antibody-Oligonucleotide Conjugate

In some embodiments, the TfR binder-oligonucleotide conjugate comprises or consists of:

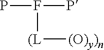

wherein
P comprises an anti-TfR antibody antigen binding domain,
F comprises a Fc dimer;
L is a linking group;
P′ comprises a non-binding Fab or NBVR;
O is an oligonucleotide;
y is an integer greater than or equal to 1 (e.g., 1, 2, 3, or 4); and
n is an integer greater than or equal to 1 (e.g., 1, 2, 3, 4, 5, 6, 7 or 8).

In some embodiments, P comprises an anti-TfR Fab (e.g., as shown in FIG. 13). In some embodiments, P comprises an anti-TfR scFv. In some embodiments, anti-TfR antibody antigen binding domain comprises an anti-TfR VHH, vNAR, or nanobody. The anti-TfR antibody antigen binding domain can be from any anti-TfR antibody known to specifically bind TfR. In some embodiments, the non-binding Fab or NVBR can be any of the non-binding Fabs or NVBRs described herein. In some embodiments, the oligonucleotide comprises an ASO. In some embodiments, the TfR binder (P, P′, and/or F) comprises at least one substitution or modification that facilitates covalent conjugation of the oligonucleotide O, optionally via the linker L. The oligonucleotide can be linked to P, F, or P′. If the oligonucleotide is linked to F (Fc dimer), it can be linked to the Fc polypeptide linked to P or the Fc polypeptide linked to P′.

C. Anti-TfR scFv-Albumin-Oligonucleotide Conjugate

In some embodiments, the TfR binder-oligonucleotide conjugate comprises or consists of:

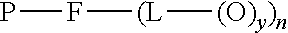

wherein
P comprises an anti-TfR antibody antigen binding domain,
F comprises albumin;
L is linking group;
O is an oligonucleotide;
y is an integer greater than or equal to 1 (e.g., 1, 2, 3, or 4); and
n is an integer greater than or equal to 1 (e.g., 1, 2, 3, 4, 5, 6, 7 or 8);

In some embodiments, P comprises an anti-TfR scFv (e.g., as shown in FIG. 13). In some embodiments, P comprises an anti-TfR Fab. In some embodiments, anti-TfR antibody antigen binding domain comprises an anti-TfR VHH, vNAR, or nanobody. The anti-TfR antibody antigen binding domain can be from any anti-TfR antibody known to specifically bind TfR. In some embodiments, the albumin is human albumen. In some embodiments, the oligonucleotide comprises an ASO. In some embodiments, the TfR binder (P and/or F) comprises at least one substitution or modification that facilitates covalent conjugation of the oligonucleotide O, optionally via the linker L.

### D. Divalent Anti-TfR Antibody (Anti-TfR (Fab) 2)-Oligonucleotide Conjugate

In some embodiments, the TfR binder-oligonucleotide conjugate comprises or consists of:

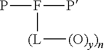

wherein
P comprises a first anti-TfR antibody antigen binding domain,
F comprises a Fc dimer;
L is a linking group;
P′ comprises a second anti-TfR antibody antigen binding domain;
O is an oligonucleotide;
y is an integer greater than or equal to 1 (e.g., 1, 2, 3, or 4); and
n is an integer greater than or equal to 1 (e.g., 1, 2, 3, 4, 5, 6, 7 or 8).

In some embodiments, P and P′ comprise an anti-TfR Fabs. In some embodiments, P and P′ comprise an anti-TfR scFvs. In some embodiments, P comprises an anti TfR Fab and P′ comprise an anti-TfR scFv. In some embodiments, P comprises an anti TfR scFv and P′ comprise an anti-TfR Fab. In some embodiments, the first and/or second anti-TfR antibody antigen binding domains comprise anti-TfR VHHs, vNARs, or nanobodies. The anti-TfR antibody antigen binding domain can be from any anti-TfR antibody known to specifically bind TfR. In some embodiments, the oligonucleotide comprises an ASO. In some embodiments, the oligonucleotide comprises an ASO. In some embodiments, the TfR binder (P, P′, and/or F) comprises at least one substitution or modification that facilitates covalent conjugation of the oligonucleotide O, optionally via the linker L.

### IV. Oligonucleotides

As described herein, one or more oligonucleotides (e.g., ASOs or RNAi agents) may be linked, optionally through a linker “L,” to a TfR binder as described herein to form a TfR binder-oligonucleotide conjugate.

While the length of the oligonucleotide may vary, in certain embodiments, the oligonucleotide is from about 10 to about 60 nucleotides in length, or from about 10 to about 30 nucleotides in length, or from about 18 to about 30 nucleotides in length or from about 15 to about 25 nucleotides in length, or from about 16 to about 20 nucleotides in length. Additionally, as described below, an oligonucleotide may comprise certain chemical modifications, such as a modified internucleoside linkage, a modified nucleobase, a modified sugar, or a combination thereof. In certain embodiments, one or more oligonucleotides are linked (i.e., through a linking group “L”) to the TfR binder. In certain embodiments, two or more oligonucleotides are linked to the TfR binder (e.g., 1, 2, 3, 4, 5, 6, 7 or 8 or more). In certain embodiments, one oligonucleotide is linked to the TfR binder. In certain embodiments, two oligonucleotides are linked to the TfR binder. In certain embodiments, four oligonucleotides are linked to the TfR binder.

In certain embodiments, 1 oligonucleotide is attached to a single linking group (L). In certain embodiments, 2 oligonucleotides are attached to a single linking group (L). For example, the oligonucleotides may be linked to each other in tandem. In certain embodiments, an L is attached at the 5′ end of a first oligonucleotide and a second oligonucleotide is linked to the 3′end of the first oligonucleotide. In certain embodiments, the oligonucleotides may be linked via a nucleic acid linker or a non-oligonucleotide cleavable linker.

In other embodiments, the linking group is a branched linking group and 2 or more oligonucleotides are attached separately to a single linking group (L) (i.e., y is 2 or more).

When two or more oligonucleotides are attached to the TfR binder, the oligonucleotides may be the same or different. In certain embodiments, the oligonucleotides are the same.

### ASOs

In one embodiment, each oligonucleotide is independently an ASO. The term “antisense oligonucleotide (ASO)” refers to single strands of DNA-like or RNA-like molecules (e.g., modified nucleotides such as those described herein) that are complementary or partially complementary to a chosen target polynucleotide sequence, e.g., an mRNA. By binding to a complementary target sequence ASOs can alter or modulate gene expression through a number of mechanisms, including, e.g., by altering splicing (exon exclusion or exon inclusion); by recruiting RNase H leading to target degradation; through translation inhibition; and by small RNA inhibition.

Typically, ASOs range from about 10 to 30 base pairs (bp) in length, but may be longer or shorter. For example, in certain embodiments, the ASO is about 10 to about 60 nucleotides in length, or about 10 to about 50 nucleotides in length, or about 10 to about 40 nucleotides in length. In certain embodiments, the ASO is about 10 to 30 nucleotides in length, or about 12 to 30 nucleotides in length, or about 14 to about 30 nucleotides in length, or about 15 to about 30 nucleotides in length, or about 16 to about 30 nucleotides in length, or about 17 to about 30 nucleotides in length, or about 18 to about 30 nucleotides in length, or about 18 to about 28 nucleotides in length or about 18 to 26 nucleotides in length, or about 18 to about 24 nucleotides in length, or about 15 to about 25 nucleotides in length, or about 16 to about 20 nucleotides in length. In certain embodiments, the ASO is about 15, 16, 17, 18, 19, 20, 21, 22, 23, 24, 25, 26, 27, 28, 29 or 30 nucleotides in length.

Selection of antisense oligonucleotide sequences specific for a given target sequence is based upon analysis of the chosen target sequence and determination of a number of factors, including secondary structure, Tₘ, binding energy, and relative stability. Additionally, antisense oligonucleotides may be selected based upon their relative inability to form dimers, hairpins, or other secondary structures that would reduce or prohibit specific binding to the target mRNA in a host cell. Target regions of the mRNA include those regions at or near the AUG translation initiation codon and those sequences that are substantially complementary to 5′ regions of the mRNA. Secondary structure analyses and target site selection considerations can be performed using software and algorithms known in the art, for example, using v.4 of the OLIGO primer analysis software (Molecular Biology Insights) and/or the BLASTN 2.0.5 algorithm software (Altschul et al, Nucleic Acids Res. 1997, 25 (17): 3389-402).

### RNAi Agents

In certain other embodiments, each oligonucleotide is independently an RNAi agent (e.g., a siRNA or shRNA). The term “RNA interference (RNAi) agent” refers to an RNA agent, or a molecule that can be cleaved into an RNA agent, that can inhibit the expression of a target gene or sequence (e.g., an mRNA, tRNA or viral RNA), in a sequence specific manner (e.g., via Dicer/RISC). RNAi agents may be single or double stranded. If the RNAi agent is a single strand it can include a 5′ modification, such as one or more phosphate groups or one or more analogs of a phosphate group. In one embodiment, the RNAi agent is double stranded and comprises a sense and an antisense strand (e.g., a short interfering RNA (siRNA)).

The RNAi agent typically includes a region of sufficient homology to the target gene, and is of sufficient length, such that the RNAi agent can mediate down regulation of the target gene. Complementarity between the RNAi agent and the target sequence should be sufficient to enable the RNAi agent, or a cleavage product thereof, to direct sequence specific silencing. In certain embodiments, the RNAi agent is, or comprises a region which is, at least partially complementary to the target RNA. In certain other embodiments, the RNAi agent is, or comprises a region which is, fully complementary to the target RNA.

In some embodiments, the RNAi agent comprises an unpaired region at one or both ends of the molecule. For example, a double stranded RNAi agent may have its strands paired with an overhang, e.g., 5′ and/or 3′ overhangs, such as an overhang of 1-3 nucleotides. In certain embodiments, an RNAi agent will comprise an unpaired overhang of 1, 2, 3 or 4 nucleotides in length at each end. The overhangs can be the result of one strand being longer than the other, or the result of two strands of the same length being staggered.

Duplexed regions within an RNAi agent may vary in length, but typically range between about 5 to about 30 nucleotides in length. In certain embodiments, the duplexed regions are between about 15-60, or about 15-50, or about 15-40, or about 15-30, or about 15-25, or about 19-25 nucleotides in length. In certain embodiments, the duplexed regions are between about 20-24, or about 21-23 nucleotides in length. In certain embodiments, the duplexed regions are about 5, 6, 7, 8, 9, 10, 11, 12, 13, 14, 15, 16, 17, 18, 19, 20, 21, 22, 23, 24 or more nucleotides in length.

A “single strand RNAi agent” or “ssRNAi agent” as used herein is made up of a single molecule. It may include a duplexed region, formed by intra-strand pairing, e.g., it may be, or include, a hairpin or pan-handle structure. Single strand RNAi agents may be antisense with regard to the target molecule. A single strand RNAi agent may be sufficiently long that it can enter RISC and participate in RISC mediated cleavage of a target mRNA. In certain embodiments, a single strand RNAi agent is at least 10, 15, 20, 25, 30, 35, 40, or 50 nucleotides in length. In certain embodiments, it is less than 200, 100, 80 or 60 nucleotides in length.

Small hairpin RNA (shRNA) agents typically have a duplex region less than 200, 100, or 50, in length. In certain embodiments, the duplex region ranges in length from about 15-60, or about 15-50, or about 15-40, or about 15-30, or about 15-25, or about 19-25 nucleotides in length. In certain embodiments, the duplexed regions are between about 17-23, or from about 19-23, or from about 20-23, or about 21-23, or about 19 to 21 nucleotides in length. In certain embodiments, the duplex region is at least about 17, 18, 19, 29, 21, 22, 23, 24, or 25 nucleotide pairs in length. The hairpin may have a single strand overhang or terminal unpaired region. In certain embodiments, the overhangs are 2-3 nucleotides in length. In some embodiments, the overhang is at the sense side of the hairpin and in some embodiments on the antisense side of the hairpin.

A “double stranded RNAi agent” or “dsRNAi agent” as used herein, includes more than one strand in which interchain hybridization can form a duplex region within the molecule (e.g., hybridization between a sense strand and an antisense strand). In certain embodiments, the RNAi agent is sufficiently large that it can be cleaved by an endogenous molecule, such as Dicer, to produce smaller molecules.

In certain embodiments, the RNAi agent is an siRNA molecule comprising sense and an antisense strands.

As used herein, term “antisense strand” refers to the strand of an RNAi agent that is sufficiently complementary to a target polynucleotide, e.g. a target mRNA. In certain embodiments, the antisense strand of a double stranded RNAi agent is at least about 10, 11, 12, 13, 14, 15, 16 17, 18, 19, 20, 21, 22, 23, 24, 25, 30, 40, 50 or 60 nucleotides in length. In certain embodiments, the antisense strand of a double stranded RNAi agent is less than about 200, 100, or 50, nucleotides in length. In certain embodiments, the antisense strand ranges in length from about 17 to 25, or about 19 to 23, or about 19 to 21 nucleotides in length.

As used herein, term “sense strand” refers to the strand of an RNAi agent that is sufficiently complementary to the antisense strand. In certain embodiments, the sense strand of a double stranded RNAi agent is at least about 10, 11, 12, 13, 14, 15, 16 17, 18, 19, 20, 21, 22, 23, 24, 25, 30, 40, 50 or 60 nucleotides in length. In certain embodiments, the sense strand of a double stranded RNAi agent is less than about 200, 100, or 50, nucleotides in length. In certain embodiments, the sense strand ranges in length from about 17 to 25, or about 19 to 23, or about 19 to 21 nucleotides in length.

In certain embodiments, the double strand portion of a double stranded RNAi agent is at least about 10, 11, 12, 13, 14, 15, 16, 17, 18, 19, 20, 21, 22, 23, 24, 25, 30, 40, 50, or 60 nucleotides in length. In certain embodiments, the sense strand of a double stranded RNAi agent is less than about 200, 100, or 50, nucleotides in length. In certain embodiments, the sense strand ranges in length from about 17 to 25, or about 19 to 23, or about 19 to 21 nucleotides in length.

In certain embodiments, the sense and antisense strands may be chosen such that the dsRNAi agent includes an unpaired region at one or both ends of the molecule. Thus, a dsRNAi agent may contain sense and antisense strands, paired to contain an overhang, e.g., 5′ and/or 3′ overhangs of between 1, 2, 3 or 4 nucleotides in length. The overhangs can be the result of one strand being longer than the other, or the result of two strands of the same length being staggered. In certain embodiments, the dsRNAi agent comprises at least one 3′ overhang. In certain embodiments, both ends of the dsRNAi agent comprise a 3′ overhang (e.g., of 2 nucleotides in length).

Duplexed regions within a dsRNAi agent may vary in length, but typically range between about 5 to about 30 nucleotides in length. In certain embodiments, the duplex region ranges in length from about 5-60, or about 15-60 or about 15-50, or about 15-40, or about 15-30, or about 15-25, or about 19-25 nucleotides in length. In certain embodiments, the duplexed regions are between about 17-23, or from about 19-23, or from about 20-23, or about 21-23, or about 19 to 21 nucleotides in length. In certain embodiments, the duplexed regions are about 5, 6, 7, 8, 9, 10, 11, 12, 13, 14, 15, 16, 17, 18, 19, 20, 21, 22, 23, 24 or more nucleotides in length.

Methods of producing RNAi agents, such as siRNA and shRNA, are known in the art and can be readily adapted to produce an RNAi agent that targets any polynucleotide sequence. In certain embodiments, an RNAi agent is chemically synthesized. For example, oligonucleotides can be synthesized using a variety of techniques, such as those described in Usman et al., J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997).

### Illustrative Oligonucleotide Modifications

In certain embodiments, an oligonucleotide described herein may comprise at least one nucleic acid modification, such as those selected from the group consisting of a modified internucleoside linkage, a modified nucleobase, a modified sugar, and combinations thereof. Such modifications may be used to alter pharmacokinetics (improved nuclease resistance resulting in a longer half-life), pharmacodynamics (superior affinity for the target RNA), or endocytic uptake. However, many modifications preclude cleavage by RNase H, which is the desired mechanism of action for many ASOs. Thus, certain RNase H ASOs may be designed as chimeras, where different bases are a mix of different chemistries, or as gapmers, where some modifications are placed on the “wings” and not the central bases. In contrast, for RNAi agents and ASOs intended to alter mRNA splicing or translation, considerations regarding RNase H are not necessary.

Accordingly, an oligonucleotide described herein may comprise one or more nucleic acid modifications. In certain embodiments, an oligonucleotide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 40, or more modifications.

In certain embodiments, an oligonucleotide described herein comprises one or more nucleotide modifications (e.g., to the nucleobase or sugar moiety). In certain embodiments, 25% or more of the nucleotides present in the oligonucleotide are modified. In certain embodiments, 50% or more of the nucleotides present in the oligonucleotide are modified. In certain embodiments, 75% or more of the nucleotides present in the oligonucleotide are modified. In certain embodiments, 100% of the nucleotides present in the oligonucleotide are modified.

In certain embodiments, the oligonucleotide comprises one or more nucleobase modifications. In certain embodiments, the oligonucleotide comprises one or more modifications to the sugar moiety (e.g., furanosyls comprising substituents at the 2′-position, the 3′-position, the 4′-position and/or the 5′-position). In certain embodiments, substituted sugar moieties include bicyclic sugar moieties.

In certain embodiments, the nucleic acid modifications with the oligonucleotide are included in a pattern. In certain embodiments, the oligonucleotide is a gapmer. The modification pattern of a gapmer oligonucleotide generally has the formula 5′-Xₐ-Yₐ-Zₐ-3′, with Xₐ and Zₐ as flanking regions around a gap region Yₐ. In certain embodiments, the Yₐ region is a contiguous stretch of nucleotides, e.g., a region of at least 6 DNA nucleotides, which are capable of recruiting an RNAse, such as RNAse H. In certain embodiments, the Yₐ region is at least 8 DNA nucleotides. In certain embodiments, the Yₐ region is about 9 to about 15 DNA nucleotides. In certain embodiments, the Yₐ region is about 11 to about 13 DNA nucleotides. In certain embodiments, the Yₐ region is 10, 11, 12, or 13 DNA nucleotides. In certain embodiments, the gapmer binds to the target nucleic acid, at which point an RNAse is recruited and can then cleave the target nucleic acid. In certain embodiments, the Yₐ region is flanked both 5′ and 3′ by regions Xₐ and Zₐ, which comprise high-affinity modified nucleotides, e.g., one to six modified nucleotides in each of Xₐ and Zₐ. In certain embodiments, the Yₐ region is flanked both 5′ and 3′ by regions Xₐ and Zₐ, wherein Xₐ and Zₐ comprise modified nucleotides having modified sugars. In certain embodiments, each nucleotide in Xₐ and Zₐ comprises a modified nucleotide having a sugar modification. The modified nucleotide can be, but is not limited to, a 2-MOE modified nucleotide, a bicyclic nucleotide, a LNA nucleotide, or a cET modified nucleotide. In certain embodiments, the modified nucleotides are present in the 5′ and 3′ regions of the oligonucleotide, while certain modified nucleotides and/or modified linkages may or may not present in the central portion of the molecule. In certain embodiments, the modified nucleotides are present in the 5′ and 3′ regions of the oligonucleotide and certain modified nucleotides are not present in the central portion of the molecule (e.g., LNA residues are not present in the central portion); however, the central region may contain modified linkages, such as PS linkages. In certain embodiments, Xₐ and Zₐ are each independently about 3 to about 6 nucleotides in length. In certain embodiments, Xₐ and Zₐ are each independently 3, 4, or 5 nucleotides in length. In certain embodiments, Xₐ and Zₐ each comprise 3 modified nucleotides. In certain embodiments, the 3 modified nucleotides are arranged in tandem in each of Xₐ and Zₐ.

Modified nucleosides/nucleotides are known in the art and include, but are not limited to, 2′-O methyl(2′OMe) residues, 2′ O-methoxyethyl (MOE) residues, constrained nucleic acid residues (e.g., S-cEt, R-cEt, S-cMOE, and R-cMOE), peptide nucleic acid (PNA) residues, locked nucleic acid (LNA) residues, and 5-methylcytidine residues (methylated cytosine residues) (see, also, Scoles, et al., Neurol Genet April 2019, 5 (2) e323). In certain embodiments, the oligonucleotide comprises one or more MOE residues. In certain embodiments, the oligonucleotide comprises one or more OMe residues or F residues (e.g., 2′-F or 2′OMe). In certain embodiments, the oligonucleotide comprises one or more constrained (e.g., S-cEt, R-cEt, S-cMOE, and R-cMOE) and/or LNA residues. Nucleic acids are considered “locked” when they have a methylene bridge connection made between 2′-oxygen and the 4′-carbon of the ribose sugar molecule. In certain embodiments, the oligonucleotide is a morpholino (i.e., comprises certain modifications to the sugar moiety). In certain embodiments, an oligonucleotide described herein comprises one or more LNA residues and one or more 5-methylcytidine residues.

In certain embodiments, the oligonucleotide comprises one or more modifications to the internucleoside backbone (i.e., the natural phosphodiester (PO) linkage is modified). In certain embodiments, such modifications are made to, e.g., reduce nuclease activity. Thus, in certain embodiments, an oligonucleotide comprises 1, 2, 3, 4, 5, 6, 7, 8, 9, 10, 11, 12, 13, 14, 15, 16, 17, 18, 19, 20, 21, 22, 23, 24, or 25 or more modified internucleoside linkages. In certain embodiments, 25% or more of the internucleoside linkages are modified. In certain embodiments, 50% or more of the internucleoside linkages are modified. In certain embodiments, 75% or more of the internucleoside linkages are modified. In certain embodiments, 100% of the internucleoside linkages present in the oligonucleotide are modified.

Backbone modifications are known in the art and include, but are not limited, to, phosphorothioate (PS) linkages, chiral phosphorothioate linkages, phosphoroamidate linkages, mesyl phosphoramidate linkages, and phosphorodiamidate linkages, phosphorodithioate linkages, aminoalkylphosphotriester linkages, phosphotriester linkages, thiophosphate linkages, phosphonate linkages, methyl phosphonate linkages, alkyl phosphonate linkages, 3′ alkylene phosphonate linkages, chiral phosphonate linkages, 3′-amino phosphoramidate linkages, aminoalkylphosphoramidate linkages, phosphinate linkages, thionoalkylphosphonate linkages, thionophosphoramidate linkages, thionoalkyl-phosphotriester linkages, borano-phosphate linkages, morpholino linkages and peptide nucleic acid (PNA) linkages. For example, in certain embodiments, one or more (e.g., 1, 2, 3, 4, 5, 6, 7, 8, 9, 10, 11, 12, 13, 14, 15, 16, 17, 18, 19, 20, 21, 22, 23, 24, 25 or more) of the internucleoside linkages in the oligonucleotide are replaced with a phosphorothioate (PS) linkage. In certain embodiments, the oligonucleotide comprises a mix of modified and unmodified linkages. The modification at one internucleoside linkage can be independent of the modification at another internucleoside linkage. In certain embodiments, every internucleoside linkage in a MAPT ASO is a modified linkage. In certain embodiments, every internucleoside linkage in a MAPT ASO is a PS linkage. In some embodiments, every internucleoside linkage in an LPA ASO is a phosphorothioate or a mesyl phosphoramidate. In certain other embodiments, one or more (e.g., 1, 2, 3, 4, 5, 6, 7, 8, 9, 10, 11, 12, 13, 14, 15, 16, 17, 18, 19, 20, 21, 22, 23, 24, or 25 or more) of the internucleoside linkages in the oligonucleotide are replaced with a phosphorodiamidate linkage. In certain embodiments, the oligonucleotide is a phosphorodiamidate morpholino (PMO).

In certain embodiments, the internucleoside linkages are stereorandom with regard to the chiral centers (Rp and Sp). In certain other embodiments, the Rp and Sp configurations in the oligonucleotide are optimized in particular configurations.

In certain embodiments, the oligonucleotide is a gapmer comprising LNA and PS modifications. For example, in certain embodiments, the oligonucleotide is a gapmer having a modification pattern of the formula 5′-Xₐ-Yₐ-Zₐ-3′, with Xₐ and Zₐ as flanking regions around a gap region Yₐ, wherein Xₐ and Zₐ each comprise 3 LNA modified nucleotides (e.g., 3 consecutive LNA modified nucleotides), and wherein the gap region Yₐ comprises PS linkages. In some embodiments, every internucleotide linkage in the antisense oligonucleotide comprises a PS linkage. In certain embodiments, the oligonucleotide further comprises one or more 5′-methylcytidine residues. In certain embodiments, the gap region Yₐ does not comprise LNA residues

### V. Linking Group

In some embodiments, the oligonucleotide is conjugated to the TfR binder (e.g., anti-TfR antibody antigen binding domain or anti-TfR antibody) via a linker “L.” In certain embodiments, L is a linking group that joins each oligonucleotide to a TfR binder. The linking group may be any group suitable for joining an oligonucleotide to a protein or polypeptide, such as an antibody.

The linking group may be attached to any region of the TfR binder, (e.g., to the N-terminal region, to the C-terminal region, or to an amino acid within the protein, such as a cysteine residue or a glutamine residue), so long as the oligonucleotide does not prevent binding of the TfR binder to the TfR. Similarly, the linking group may be attached to any region of the oligonucleotide (e.g., the 5′ end, the 3′end or to a nucleic acid residue within the molecule), so long as the TfR binder does not interfere with the functionality of the oligonucleotide (e.g., complementary binding to a target nucleic acid). For example, the linker may be attached to the oligonucleotide through any number of synthetically feasible points located throughout the oligo, such as at the 3′ or 5′ terminal residues of the oligo; at a sugar moiety; at a base moiety; or at a residue located within the backbone.

In certain embodiments, the linker is attached to the oligonucleotide at the 5′ terminal residue of the oligonucleotide. In certain embodiments, the linker is attached to the oligonucleotide at the 3′ terminal residue of the oligonucleotide. In certain embodiments, the linker is attached to the oligonucleotide at a residue within the oligonucleotide. In certain embodiments, the oligonucleotide is a double stranded RNAi molecule, wherein the linker is attached to the sense strand (e.g., at the 5′ or 3′ terminal residue). In certain embodiments, the oligonucleotide is a double stranded RNAi molecule, wherein the linker is attached to the antisense strand (e.g., at the 5′ or 3′ terminal residue). In certain embodiments, the oligonucleotide is siRNA, wherein the linker is attached to the 3′ end of the sense strand. In certain embodiments, the 3′ end of the sense strand of the siRNA is modified with a C6 amine.

In certain embodiments, the linking group comprises at least one spacer. In certain embodiments, the spacer is a hydrophilic spacer. In certain embodiments, the hydrophilic spacer is a polyethylene glycol (PEG).

The linking group can be a homobifuctional linker or a heterobifunctional linker.

In some embodiments, the linking group is cleavable (e.g., a nuclease-cleavable linker, an acid-labile linker, peptidase-sensitive linker, photolabile linker, dimethyl linker or disulfide-containing linker (Chari et al., Cancer Res. 52:127-131 (1992); U.S. Pat. No. 5,208,020). In certain embodiments, the linking group comprises one or more nucleotides (e.g., 1, 2, 3, or more) or one or more nucleosides (e.g., 1, 2, 3, or more). In certain embodiments, the one or more nucleotides or one or more nucleosides of the linking group are unmodified. In certain embodiments, the linking group comprises one or more nucleotides having unmodified bases, unmodified sugar groups and/or unmodified phosphate groups. In certain embodiments, the linking group comprises one or more nucleosides having unmodified bases and/or unmodified sugar groups. In certain embodiments, the linking group comprises a TCA (thymine-cytosine-adenine) trinucleotide. In certain embodiments, the TCA is modified with a C6 amine at T Position. In certain embodiments, the linking group does not comprise TCA.

In certain embodiments, the linking group is enzymatically cleavable. In certain embodiments, the linking group is cleavable by an enzyme present in the central nervous system (CNS) or muscle. In certain embodiments, a cleavable linking group is suitable for conjugates comprising ASOs (e.g., to enable the ASO to dissociate from the remainder of the conjugate for transport into the nucleus). In certain embodiments, the cleavable linking group is a cleavable dipeptide linker. In certain embodiments, the cleavable dipeptide linker is a valine-citrulline cleavable linking group or valine-alanine cleavable linker.

In certain embodiments, the cleavable linking group is an acid cleavable linker. In certain embodiments, the acid cleavable linker is a carbonate linker or a hydrazone linker.

In certain embodiments, the cleavable linking group comprises one or more PEG spacers.

In certain embodiments, the cleavable linking group is a disulfide such as SPDP (succinimidyl 3-(2-pyridyldithio) propionate) or lys-conjugated acid-cleavable hydrazide.

In certain embodiments, the linking group is a non-cleavable linking group. In certain embodiments, the linking group is a covalent linking group. In certain embodiments, the covalent linking group is derivable from an 3-Arylpropiolonitriles (APN) or an acrylamide. In certain embodiments, the covalent linking group comprises a group —CH₂CH₂C(═O)—. In certain embodiments, the covalent linking group comprises a group:

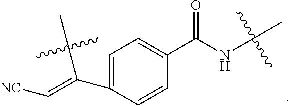 [SMILES: *NC(=O)c1ccc(/C(*)=C/C#N)cc1]

In certain embodiments, the covalent linking group is derivable from a haloacetamide, e.g., bromoacetamide, chloroacetamide, iodoacetamide.

In certain embodiments, the linking group comprises a C₆ amine group having the formula —(CH₂)₆—NH—.

In certain embodiments, the linking group is derivable from a maleimide. For example, in certain embodiments, the linking group comprises a group:

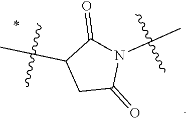 [SMILES: *C1CC(=O)N(*)C1=O]

In certain embodiments, the linking group may be attached to P at the valence marked * (e.g., to a sulfur atom of a modified site within P).

In certain embodiments, the maleimide is a modified maleimide. In certain embodiments, the modified maleimide is an alkyl-, aryl-, cycloalkyl-, or exocyclic-maleimide.

In certain embodiments, the linking group is a self-hydrolyzing linking group.

Certain specific, non-limiting embodiments of exemplary linking groups (abbreviated as Linker Embodiments LE1-LE42) are described below.

In Linker Embodiment LE1, the linking group has a molecular weight of from about 20 daltons to about 5,000 daltons. In Linker Embodiment LE2, the linking group has a molecular weight of from about 20 daltons to about 1,000 daltons. In Linker Embodiment LE3, the linking group has a molecular weight of from about 20 daltons to about 200 daltons.

In Linker Embodiment LE4, the linking group has a length of about 5 angstroms to about 60 angstroms. In Linker Embodiment LE5, the linking group separates the oligonucleotide from the TfR binder of formula (I) by about 5 angstroms to about 40 angstroms, inclusive, in length.

In Linker Embodiment LE6, the linking group is a divalent, branched or unbranched, saturated or unsaturated, hydrocarbon chain, having from 2 to 25 carbon atoms, wherein one or more (e.g., 1, 2, 3, or 4) of the carbon atoms is optionally replaced by (—O—), (—NH—), (—S—), an amino acid, a hydrazone (—C(R′)═N═N(R′)—), a nucleotide, or a 3-12 membered divalent heterocycle, wherein the chain and any 3-12 membered di-valent heterocycle is optionally substituted with one or more (e.g., 1, 2, 3, or 4) substituents independently selected from the group consisting of (C₁-C₆)alkoxy, (C₃-C₆) cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), a hydrazone (═N═N(R′)—) carboxy, aryl, aryloxy, heteroaryl, and heteroaryloxy; wherein each R′ is independently H or (C₁-C₆)alkyl.

In Linker Embodiment LE7, the linking group is a divalent, branched or unbranched, saturated or unsaturated, hydrocarbon chain, having from 2 to 25 carbon atoms, wherein one or more (e.g., 1, 2, 3, or 4) of the carbon atoms is optionally replaced by (—O—), (—NH—), or a 3-12 membered di-valent heterocycle, wherein the chain and any 3-12 membered di-valent heterocycle is optionally substituted with one or more (e.g., 1, 2, 3, or 4) substituents independently selected from the group consisting of (C₁-C₆)alkoxy, (C₃-C₆) cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In Linker Embodiment LE8, the linking group is a divalent, branched or unbranched, saturated or unsaturated, hydrocarbon chain, having from 2 to 10 carbon atoms, wherein one or more (e.g., 1, 2, 3, or 4) of the carbon atoms is optionally replaced by (—O—), (—NH—), (—S—), an amino acid, a hydrazone (—C(R′)═N═N(R′)—), a nucleotide, or a 3-12 membered di-valent heterocycle, wherein the chain and any 3-12 membered di-valent heterocycle is optionally substituted with one or more (e.g., 1, 2, 3, or 4) substituents independently selected from the group consisting of (C₁-C₆)alkoxy, (C₃-C₆) cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), a hydrazone (═N═N(R′)—) carboxy, aryl, aryloxy, heteroaryl, and heteroaryloxy; wherein each R′ is independently H or (C₁-C₆)alkyl.

In Linker Embodiment LE9, the linking group is a divalent, branched or unbranched, saturated or unsaturated, hydrocarbon chain, having from 2 to 10 carbon atoms, wherein one or more (e.g., 1, 2, 3, or 4) of the carbon atoms is optionally replaced by (—O—), (—NH—), or a 3-12 membered di-valent heterocycle, wherein the chain and any 3-12 membered di-valent heterocycle is optionally substituted with one or more (e.g., 1, 2, 3, or 4) substituents independently selected from the group consisting of (C₁-C₆)alkoxy, (C₃-C₆) cycloalkyl, (C₁—C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In Linker Embodiment LE10, the linking group is a divalent, branched or unbranched, saturated or unsaturated, hydrocarbon chain, having from 2 to 25 carbon atoms, wherein the chain is optionally substituted on carbon with one or more (e.g., 1, 2, 3, or 4) substituents selected from (C₁-C₆)alkoxy, (C₃-C₆) cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In Linker Embodiments LE11, the linking group is a divalent, branched or unbranched, saturated or unsaturated, hydrocarbon chain, having from 2 to 10 carbon atoms, wherein the chain is optionally substituted on carbon with one or more (e.g. 1, 2, 3, or 4) substituents selected from (C₁-C₆)alkoxy, (C₃-C₆) cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In Linker Embodiment LE12, the linking group is a divalent, branched or unbranched, saturated or unsaturated, hydrocarbon chain, having from 2 to 10 carbon atoms.

In Linker Embodiment LE13, the linking group is a divalent, branched or unbranched, saturated hydrocarbon chain, having from 2 to 10 carbon atoms.

In Linker Embodiment LE14, the linking group is a divalent, unbranched, saturated hydrocarbon chain, having from 2 to 10 carbon atoms.

In Linker Embodiment LE15, the linking group is a divalent, branched or unbranched, saturated or unsaturated, chain having from 2 to 25 atoms selected from carbon, oxygen, nitrogen and sulfur, where in the chain comprises one or more disulfide linkages.

In Linker Embodiment LE16, the linking group is a divalent, branched or unbranched, saturated or unsaturated, chain having from 2 to 25 atoms selected from carbon, oxygen, nitrogen and sulfur, where in the chain comprises one or more hydrazone groups in the chain or appended to a carbon atom of the chain.

In Linker Embodiment LE17, the linking group is a divalent, branched or unbranched, saturated or unsaturated, chain having from 2 to 35 atoms selected from carbon, oxygen, nitrogen and sulfur, where in the chain comprises one or more amino acids in the chain.

In Linker Embodiment LE18, the linking group is a divalent, branched or unbranched, saturated or unsaturated, chain having from 2 to 35 atoms selected from carbon, oxygen, nitrogen and sulfur, where in the chain comprises a dipeptide in the chain.

In Linker Embodiment LE19, the linking group is a divalent, branched or unbranched, saturated or unsaturated, chain having from 2 to 35 atoms selected from carbon, oxygen, nitrogen and sulfur, where in the chain comprises the dipeptide valine-citrulline in the chain.

In Linker Embodiment LE20, the linking group comprises one or more nucleotides in the chain.

In Linker Embodiment LE21, the linking group comprises two or more nucleotides in the chain.

In Linker Embodiment LE22, the linking group comprises a tri-nucleotide group in the chain.

In Linker Embodiment LE23, at least one linking group is attached to two or more oligonucleotides (e.g., for a compound of formula (I) y is greater than or equal to 2 for at least one (L-(O)_{y})).

In Linker Embodiment LE24, only one linking group is attached to two or more oligonucleotides (e.g., for a compound of formula (I) one y is greater than or equal to 2 for a single (L-(O)_{y})).

In Linker Embodiment LE24b, the TfR binder-oligonucleotide conjugate contains a single linking group which is attached to two or more oligonucleotides (e.g., for a compound of formula (I), n=1 and y is greater than or equal to 2).

In Linker Embodiment LE25, at least two linking groups are attached to two or more oligonucleotides (e.g., for a compound of formula (I) n is greater than or equal to 2 and y is greater than or equal to 2 for at least two (L-(O)_{y})).

In Linker Embodiment LE26, at least two linking groups are attached to two oligonucleotides (e.g., for a compound of formula (I), n is greater than or equal to 2 and y=2 for at least two (L-(O)_{y})).

In Linker Embodiment LE27, the linking group is attached to the oligonucleotide through a phosphate of the oligonucleotide (e.g., associated with the 5′ terminal residue).

In Linker Embodiment LE28, the linking group is attached to the oligonucleotide through a phosphorothioate of the oligonucleotide (e.g., associated with the 5′ terminal residue).

In Linker Embodiment LE29, the linking group comprises a polyethyleneoxy chain. In another embodiment of the invention the polyethyleneoxy chain comprises 2, 3, 4, 5, 6, 7, 8, 9, or 10 repeating ethyleneoxy units.

In Linker Embodiment LE30, the linking group comprises a 5-membered divalent heterocycle.

In Linker Embodiment LE31, the linking group has the following structure:

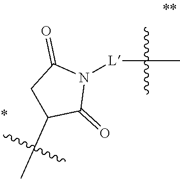

wherein L′ is a divalent, branched or unbranched, saturated or unsaturated, hydrocarbon chain, having from 2 to 25 carbon atoms, wherein one or more (e.g., 1, 2, 3, or 4) of the carbon atoms is optionally replaced by (—O—), (—NH—), (—S—), an amino acid, a hydrazone (—C(R′)═N═N(R′)—), a nucleotide, or a 3-12 membered di-valent heterocycle, wherein the chain and any 3-12 membered di-valent heterocycle is optionally substituted with one or more (e.g., 1, 2, 3, or 4) substituents independently selected from the group consisting of (C₁-C₆)alkoxy, (C₃-C₆) cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (—O), a hydrazone (—NH—N═C(R′)—), carboxy, aryl, aryloxy, heteroaryl, and heteroaryloxy; wherein each R′ is independently H or (C₁-C₆)alkyl; and wherein the valence marked * is attached to P and the valence marked ** is attached to O in formula (I). In another embodiment, L′ is a divalent, branched or unbranched, saturated or unsaturated, chain having from 2 to 25 atoms selected from carbon, oxygen, nitrogen and sulfur, where in the chain comprises one or more disulfide linkages. In another embodiment, L′ is a divalent, branched or unbranched, saturated or unsaturated, chain having from 2 to 25 atoms selected from carbon, oxygen, nitrogen and sulfur, where in the chain comprises one or more hydrazone groups in the chain or appended to a carbon atom of the chain. In another embodiment, L′ is a divalent, branched or unbranched, saturated or unsaturated, chain having from 2 to 35 atoms selected from carbon, oxygen, nitrogen and sulfur, where in the chain comprises one or more amino acids in the chain. In another embodiment, L′ is a divalent, branched or unbranched, saturated or unsaturated, chain having from 2 to 35 atoms selected from carbon, oxygen, nitrogen and sulfur, where in the chain comprises a dipeptide in the chain. In another embodiment, L′ is a divalent, branched or unbranched, saturated or unsaturated, chain having from 2 to 35 atoms selected from carbon, oxygen, nitrogen and sulfur, where in the chain comprises the dipeptide valine-citrulline in the chain. In another embodiment, L′ comprises one or more nucleotides. In another embodiment, L′ comprises two or more nucleotides. In another embodiment, L′ comprises a tri-nucleotide group. In another embodiment, L′ comprises one or more nucleotides having unmodified bases, unmodified sugar groups and/or unmodified phosphate groups.

In Linker Embodiment LE32, L′ has the following structure:

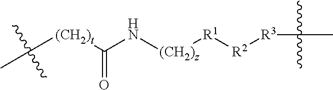

wherein t is 1, 2, 3, 4, 5, 6, 7, or 8; z is 0, 1, 2, 3, 4, 5, 6, 7, or 8; and each of R¹, R², and R³ is independently a nucleotide.

In Linker Embodiment LE33, L′ has the following structure:

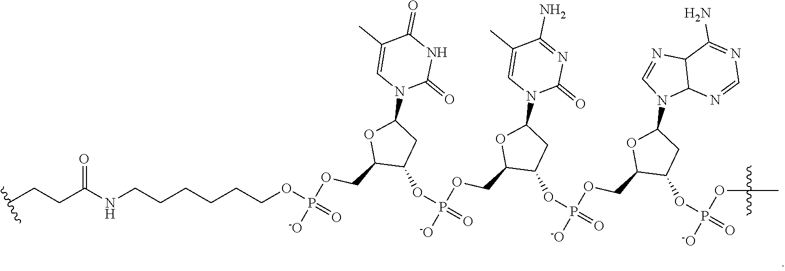

In Linker Embodiment LE34, the linking group has the following structure:

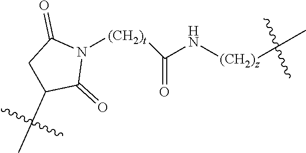

wherein t is 1, 2, 3, 4, 5, 6, 7, or 8; and z is 0, 1, 2, 3, 4, 5, 6, 7, or 8.

In Linker Embodiment LE35, the linking group has the following structure:

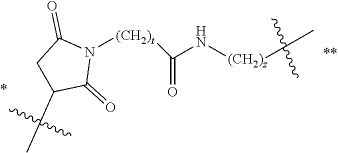

wherein t is 1, 2, 3, 4, 5, 6, 7, or 8; and z is 0, 1, 2, 3, 4, 5, 6, 7, or 8, wherein the valence marked * is attached to P and the valence marked ** is attached to O in formula (I). In certain embodiments, the valence marked ** is attached to O through a phosphate of the oligonucleotide (e.g., associated with the 5′ terminal residue).

In Linker Embodiment LE36, the linking group has the following structure:

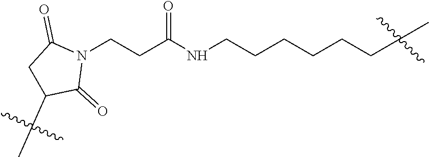 [SMILES: *CCCCCCNC(=O)CCN1C(=O)CC(*)C1=O]

In Linker Embodiment LE37, the linking group has the following structure:

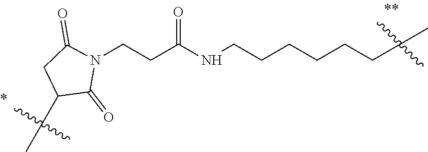 [SMILES: *CCCCCCNC(=O)CCN1C(=O)CC(*)C1=O]

wherein the valence marked * is attached to P and the valence marked ** is attached to O in formula (I). In certain embodiments, the valence marked ** is attached to O through a phosphate of the oligonucleotide (e.g., associated with the 5′ terminal residue). As such, the A group in the linker structures can, in embodiments, be covalently bound to —O—PO₃ at

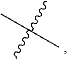

which is itself covalently bound to the oligonucleotide.

In Linker Embodiment LE38, the linking group has the following structure:

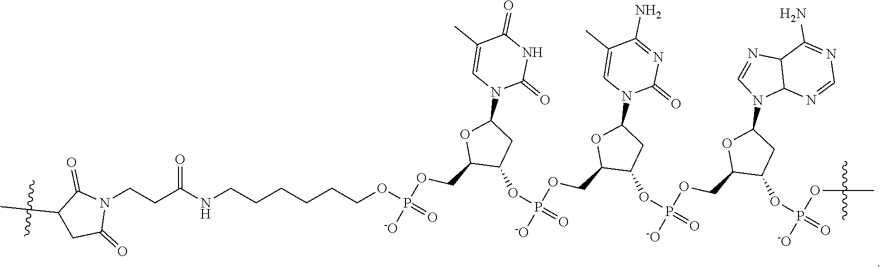

In Linker Embodiment LE39, the linker is a peptide linker or formed from a protein, peptide or amino acid. For example, in certain embodiments, the linking group is a divalent radical formed from a protein. In another embodiment, the linking group is a divalent radical formed from a peptide. In another embodiment, the linking group is a divalent radical formed from an amino acid.

In Linker Embodiment LE40, the linking group may be configured such that it allows for the rotation of the oligonucleotide and the TfR binder relative to each other; and/or is resistant to digestion by proteases. In some embodiments, the linking group may be a flexible linker, e.g., containing amino acids such as Gly, Asn, Ser, Thr, Ala, and the like. Such linking groups are designed using known parameters. For example, the linking groups may have repeats, such as Gly-Ser repeats.

In Linker Embodiment LE41, the linking group has or comprises a formula selected from the group consisting of:

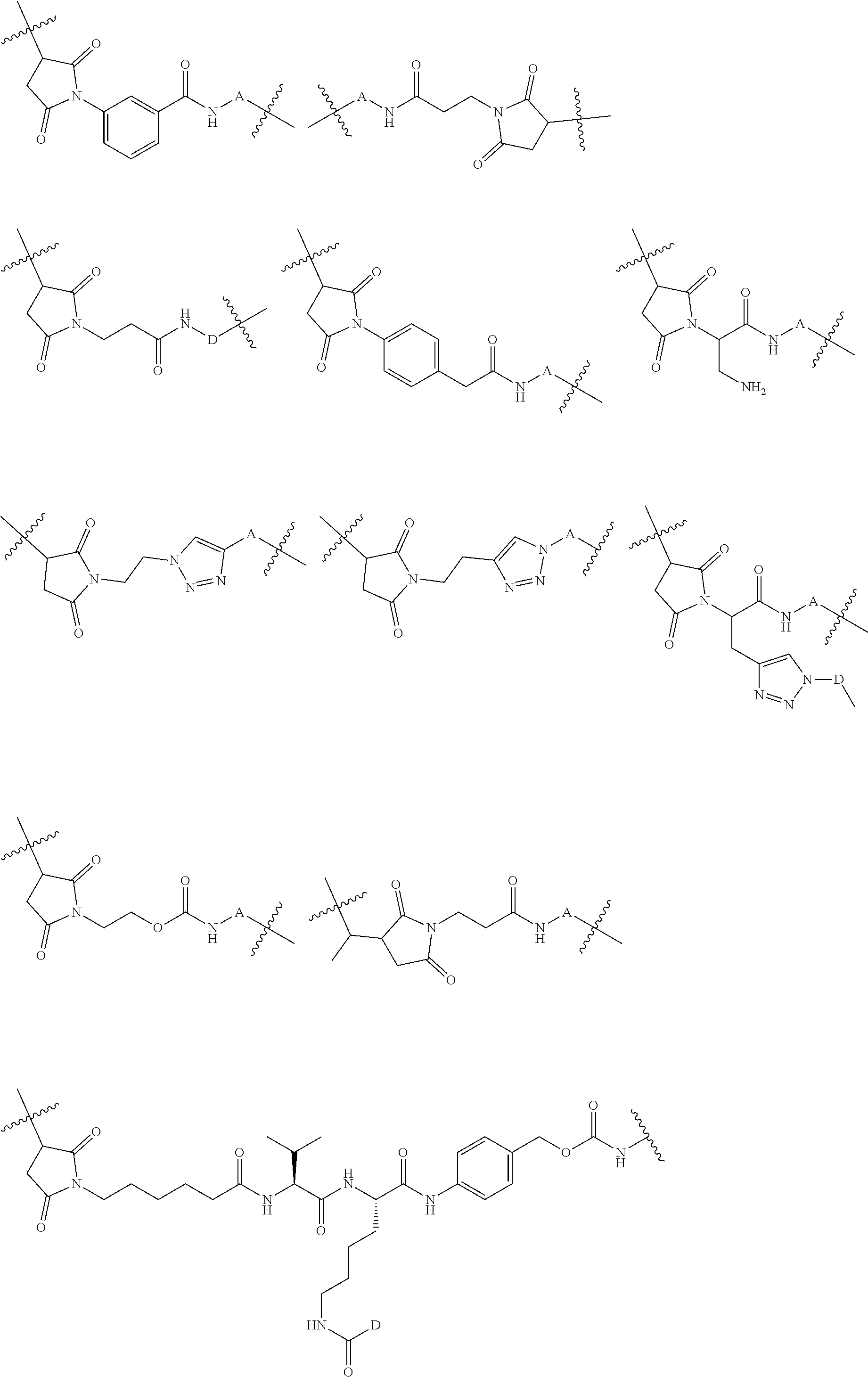

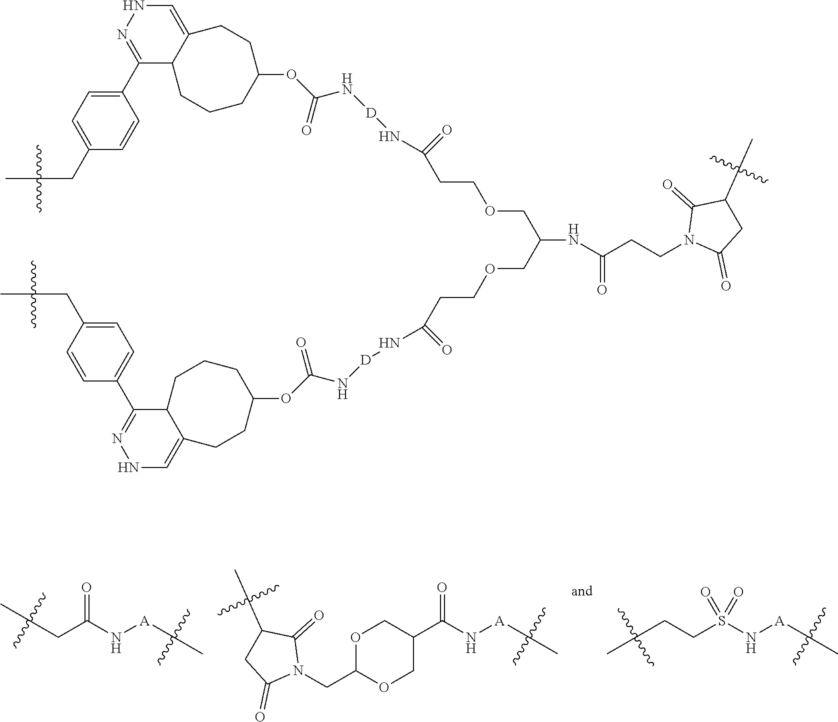

wherein
each A is independently (C₁-C₁₅) alkyl;
each D is —(CH₂—CH₂—O)ₘ—; and
each m is 1, 2, 3, 4, 5, 6, 7, 8, 9, 10, 11, 12, 13, 14, 15, 16, 17, 18, 19, 20, 21, 22, 23, or 24.

In Linker Embodiment LE42, the linking group has or comprises a formula selected from the group consisting of:

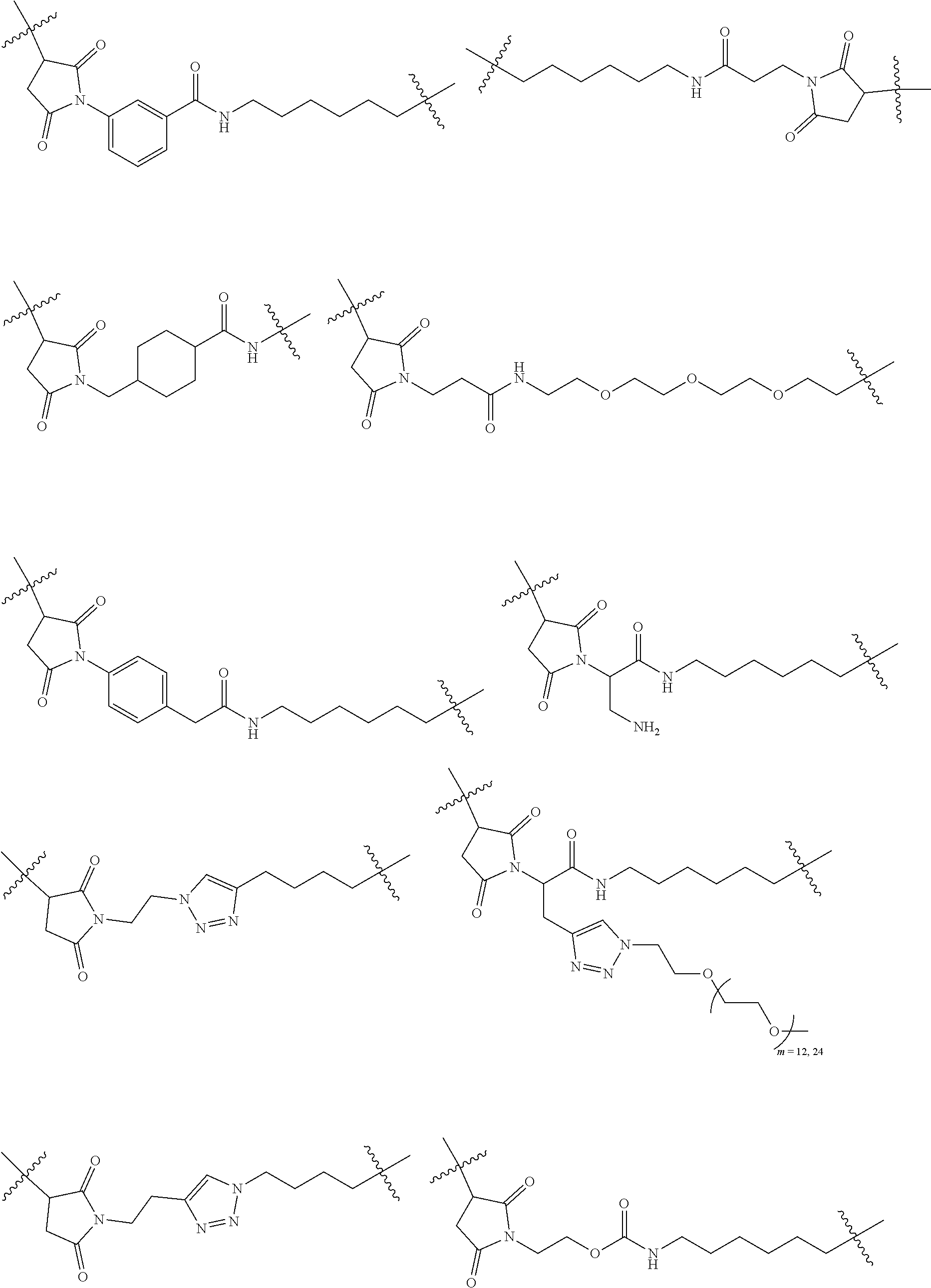

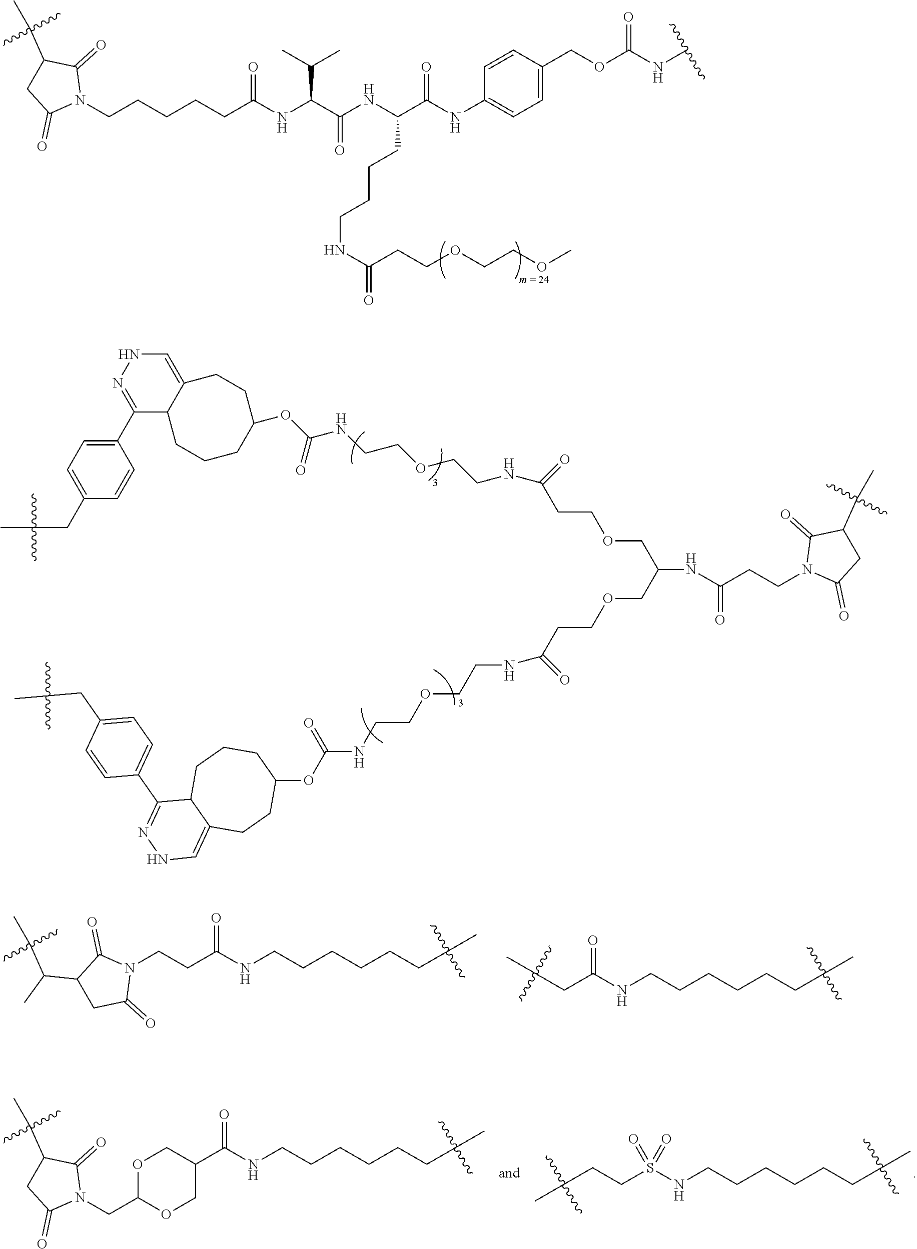

In various embodiments, the conjugates can be generated using well-known chemical cross-linking reagents and protocols. For example, there are a large number of chemical cross-linking agents that are known to those skilled in the art and useful for cross-linking a protein with an agent of interest. For example, the cross-linking agents are heterobifunctional cross-linkers, which can be used to link molecules in a stepwise manner. Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art, including N-hydroxysuccinimide (NHS) or its water soluble analog N-hydroxysulfosuccinimide (sulfo-NHS), succinimidyl 4-(N-maleimidomethyl)cyclohexane-1-carboxylate (SMCC), m-maleimidobenzoyl-N-hydroxysuccinimide ester (MBS); N-succinimidyl (4-iodoacetyl)aminobenzoate (SIAB), succinimidyl 4-(p-maleimidophenyl) butyrate (SMPB), 1-ethyl-3-(3-dimethylaminopropyl) carbodiimide hydrochloride (EDC); 4-succinimidyloxycarbonyl-a-methyl-α-(2-pyridyldithio)-toluene (SMPT), N-succinimidyl 3-(2-pyridyldithio) propionate (SPDP), and succinimidyl 6-[3-(2-pyridyldithio) propionate]hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives to reduce the amount of linker cleavage in vivo. In addition to the heterobifunctional cross-linkers, there exist a number of other cross-linking agents including homobifunctional and photoreactive cross-linkers. Disuccinimidyl subcrate (DSS), bismaleimidohexane (BMH) and dimethylpimelimidate·2HCl (DMP) are examples of useful homobifunctional cross-linking agents, and bis-[B-(4-azidosalicylamido)ethyl]disulfide (BASED) and N-succinimidyl-6 (4′-azido-2′-nitrophenylamino) hexanoate (SANPAH) are examples of useful photoreactive cross-linkers.

### VI. Anti-TfR Antibody Antigen Binding Domains

An anti-TfR antibody antigen binding domain suitable for use in the described TfR binder-oligonucleotide conjugates can be an anti-TfR antibody antigen binding domain derived from an antibody known to specifically bind TfR. Anti-TfR antibody antigen binding domain can be derived from, but is not limited to, any of the antibodies or protein molecules described in US20130028891, US2018282408, US20190092870, US2020071413, US20210138083, WO2014/033074, WO2015/101588, WO2016/081640, WO2016/208695, WO2018/124121, WO2018/210898, WO2020/132584, WO2021/076546, WO2021/205358, WO2022/101633, WO2022/103769, WO2022/221505, Candelaria et al. (Front. Immunol. 12 17 Mar. 2021, 2021), and Weber et al. (Cell Reports 22:149-162, 2018), each of which is incorporated herein by reference in its entirety. The anti-TfR antibody antigen binding domain can comprise an antibody, a Fab (including a F(ab′)2), a scFab, a Fv fragment, an scFv, a VHH, vNAR, or a nanobody.

In some embodiments, a TfR binder comprises an antibody having at least one variable domain or antigen binding site that specifically binds TfR. In some embodiments, a TfR binder comprises an antibody having a single variable domain or antigen binding site that specifically binds TfR. In some embodiments, a TfR binder comprises an antibody having a single variable domain or antigen binding site that specifically binds TfR (is monovalent, i.e., wherein the TfR-binder does not comprise any additional antibody antigen binding domain, non-binding Fab, or NBVR (anti-TfR mono-Fab). In some embodiments, a TfR binder comprises a bispecific divalent antibody having a first variable domain or antigen binding site that specifically binds TfR and a second variable domain or antigen binding site comprising a non-binding Fab or NBVR. In some embodiments, a TfR binder comprises an anti-TfR antibody binding domain (e.g., a anti-TfR Fab, scFv, VHH, vNAR, or nanobody) linked to an albumin (e.g., a human albumin). In some embodiments, a TfR binder comprises an anti-TfR antibody having a first anti-TfR antibody antigen binding domain (e.g., Fab or scFv) and a second anti-TfR antibody antigen binding domain (e.g., Fab or scFv).

Illustrative Proteins Comprising Fabs that Specifically Binds TfR

Exemplary Fabs that specifically binds to a TfR include the heavy chain variable region of SEQ ID NO: 10, 19, 102, 104, 110, 122, 132, or 143 and the light chain variable region of SEQ ID NO: 9, 18, 103, 105, 111, 123, 133, or 144. Unless otherwise apparent from context, reference to a Fab that specifically binds to a TfR should be understood as referring to any of mouse, chimeric, veneered, humanized, and modified forms.

In some embodiments, a Fab that specifically binds to a TfR comprises the heavy chain variable region of SEQ ID NO: 10 and the light chain variable region of SEQ ID NO: 9. In some embodiments, a Fab that specifically binds to a TfR comprises the heavy chain variable region of SEQ ID NO: 19 and the light chain variable region of SEQ ID NO: 18. In some embodiments, a Fab that specifically binds to a TfR comprises the heavy chain variable region of SEQ ID NO: 102 and the light chain variable region of SEQ ID NO: 103. In some embodiments, a Fab that specifically binds to a TfR comprises the heavy chain variable region of SEQ ID NO: 104 and the light chain variable region of SEQ ID NO: 105. In some embodiments, a Fab that specifically binds to a TfR comprises the heavy chain variable region of SEQ ID NO: 110 and the light chain variable region of SEQ ID NO: 111. In some embodiments, a Fab that specifically binds to a TfR comprises the heavy chain variable region of SEQ ID NO: 122 and the light chain variable region of SEQ ID NO: 123. In some embodiments, a Fab that specifically binds to a TfR comprises the heavy chain variable region of SEQ ID NO: 132 and the light chain variable region of SEQ ID NO: 133. In some embodiments, a Fab that specifically binds to a TfR comprises the heavy chain variable region of SEQ ID NO: 143 and the light chain variable region of SEQ ID NO: 144.

In some embodiments, a Fab that specifically binds to a TfR consists of the heavy chain variable region of SEQ ID NO: 10 and the light chain variable region of SEQ ID NO: 9. In some embodiments, a Fab that specifically binds to a TfR consists of the heavy chain variable region of SEQ ID NO: 19 and the light chain variable region of SEQ ID NO: 18. In some embodiments, a Fab that specifically binds to a TfR consists of the heavy chain variable region of SEQ ID NO: 102 and the light chain variable region of SEQ ID NO: 103. In some embodiments, a Fab that specifically binds to a TfR consists of the heavy chain variable region of SEQ ID NO: 104 and the light chain variable region of SEQ ID NO: 105. In some embodiments, a Fab that specifically binds to a TfR consists of the heavy chain variable region of SEQ ID NO: 110 and the light chain variable region of SEQ ID NO: 111. In some embodiments, a Fab that specifically binds to a TfR consists of the heavy chain variable region of SEQ ID NO: 122 and the light chain variable region of SEQ ID NO: 123. In some embodiments, a Fab that specifically binds to a TfR consists of the heavy chain variable region of SEQ ID NO: 132 and the light chain variable region of SEQ ID NO: 133. In some embodiments, a Fab that specifically binds to a TfR consists of the heavy chain variable region of SEQ ID NO: 143 and the light chain variable region of SEQ ID NO: 144.

In some embodiments, a Fab that specifically binds to a TfR comprises the heavy chain CH1 and variable regions of SEQ ID NO: 10 and a light chain comprising SEQ ID NO: 9. In some embodiments, a Fab that specifically binds to a TfR comprises the heavy chain CH1 and variable regions of SEQ ID NO: 19 and a light chain comprising SEQ ID NO: 18. In some embodiments, a Fab that specifically binds to a TfR comprises a heavy chain comprising SEQ ID NO: 102 and a light chain comprising SEQ ID NO: 103. In some embodiments, a Fab that specifically binds to a TfR comprises a heavy chain comprising SEQ ID NO: 104 and a light chain comprising SEQ ID NO: 105. In some embodiments, a Fab that specifically binds to a TfR comprises a heavy chain comprising SEQ ID NO: 110 and a light chain comprising SEQ ID NO: 111. In some embodiments, a Fab that specifically binds to a TfR comprises a heavy chain comprising SEQ ID NO: 122 and a light chain comprising SEQ ID NO: 123. In some embodiments, a Fab that specifically binds to a TfR comprises a heavy chain comprising SEQ ID NO: 132 and a light chain comprising SEQ ID NO: 133. In some embodiments, a Fab that specifically binds to a TfR comprises a heavy chain comprising SEQ ID NO: 143 and a light chain comprising SEQ ID NO: 144.

In some embodiments, a Fab that specifically binds to a TfR consists of the heavy chain CH1 and variable regions of SEQ ID NO: 10 and the light chain of SEQ ID NO: 9. In some embodiments, a Fab that specifically binds to a TfR consists of the heavy chain CH1 and variable regions of SEQ ID NO: 19 and the light chain of SEQ ID NO: 18. In some embodiments, a Fab that specifically binds to a TfR consists of SEQ ID NO: 102 and SEQ ID NO: 103. In some embodiments, a Fab that specifically binds to a TfR consists of SEQ ID NO: 104 and SEQ ID NO: 105. In some embodiments, a Fab that specifically binds to a TfR consists of SEQ ID NO: 110 and SEQ ID NO: 111. In some embodiments, a Fab that specifically binds to a TfR consists of SEQ ID NO: 122 and SEQ ID NO: 123. In some embodiments, a Fab that specifically binds to a TfR consists of SEQ ID NO: 132 and SEQ ID NO: 133. In some embodiments, a Fab that specifically binds to a TfR consists of SEQ ID NO: 143 and SEQ ID NO: 144.

In some embodiments, a Fab that specifically binds to a TfR comprises the CDR sequences of SEQ ID NOs: 10 and 9, 19 and 18, 102 and 103, 104 and 105, 110 and 111, 122 and 123, 132 and 133; or 143 and 144.

In some embodiments, a Fab that specifically binds to a TfR comprises CDR-H1, CDR-H2, CDR-H3, CDR-L1, CDR-L2, and CDR-L3 having the sequences of SEQ ID NOs: 12, 13, 14, 15, 16, and 17, respectively. In some embodiments, a Fab that specifically binds to a TfR comprises CDR-H1, CDR-H2, CDR-H3, CDR-L1, CDR-L2, and CDR-L3 having the sequences of SEQ ID NOs: 21, 22, 23, 24, 25, and 26, respectively. In some embodiments, a Fab that specifically binds to a TfR comprises CDR-H1, CDR-H2, CDR-H3, CDR-L1, CDR-L2, and CDR-L3 having the sequences of SEQ ID NOs: 114, 115, 116, 117, 118, and 119, respectively. In some embodiments, a Fab that specifically binds to a TfR comprises CDR-H1, CDR-H2, CDR-H3, CDR-L1, CDR-L2, and CDR-L3 having the sequences of SEQ ID NOs: 126, 127, 128, 129, 130, and 131, respectively. In some embodiments, a Fab that specifically binds to a TfR comprises CDR-H1, CDR-H2, CDR-H3, CDR-L1, CDR-L2, and CDR-L3 having the sequences of SEQ ID NOs: 134, 135, 136, 137, 138, and 139, respectively. In some embodiments, a Fab that specifically binds to a TfR comprises CDR-H1, CDR-H2, CDR-H3, CDR-L1, CDR-L2, and CDR-L3 having the sequences of SEQ ID NOs: 154, 155, 156, 157, 158, and 159 respectively. In some embodiments, a Fab that specifically binds to a TfR comprises CDR-H1, CDR-H2, CDR-H3, CDR-L1, CDR-L2, and CDR-L3 having the sequences of SEQ ID NOs: 161, 162, 163, 164, 165, and 166, respectively.

In some embodiments, a Fab that specifically binds to a TfR comprises a light chain containing an amino acid sequence that is at least 70%, at least 80%, at least 90%, at least 95%, at least 96%, at least 97%, at least 98%, or at least 99% identical to the amino acid sequence of SEQ ID NOs: 9, 18, 103, 105, 111, 123, 133, or 144, and a heavy chain containing an amino acid sequence that is at least 70%, at least 80%, at least 90%, at least 95%, at least 96%, at least 97%, at least 98%, or at least 99% identical to the VH and CH1 region amino acid sequence of SEQ ID NOs: 10, 19, 102, 104, 110, 122, 132, or 143, and contains the CDR sequences of the SEQ ID NOs: 9 and 10, 18 and 19, 102 and 103, 104 and 105, 110 and 111, 122 and 123, 132 and 133; or 143 and 144.

In some embodiments, a Fab that specifically binds to a TfR comprises light chain and heavy chain variably regions that differ from the variable regions of SEQ ID NOs: 9 and 10, 18 and 19, 102 and 103, 104 and 105, 110 and 111, 122 and 123, 132 and 133; or 143 and 144 by a small number of functionally inconsequential amino acid substitutions (e.g., conservative substitutions), deletions, or insertions.

In some embodiments, a Fab-Fc fusion comprises SEQ ID NO: 10 and SEQ ID NO: 9. In some embodiments, a Fab-Fc fusion comprises SEQ ID NO: 19 and SEQ ID NO: 18. In some embodiments, a Fab-Fc fusion comprises SEQ ID NO: 102 and SEQ ID NO: 103. In some embodiments, a Fab-Fc fusion comprises SEQ ID NO: 104 and SEQ ID NO: 105. In some embodiments, a Fab-Fc fusion comprises SEQ ID NO: 110 and SEQ ID NO: 111. In some embodiments, a Fab-Fc fusion comprises SEQ ID NO: 122 and SEQ ID NO: 123. In some embodiments, a Fab-Fc fusion comprises SEQ ID NO: 132 and SEQ ID NO: 133. In some embodiments, a Fab-Fc fusion comprises SEQ ID NO: 143 and SEQ ID NO: 144.

In some embodiments, a Fab-Fc fusion comprises amino acid sequences at least 70%, at least 80%, at least 90%, at least 95%, at least 96%, at least 97%, at least 98%, or at least 99% identical to the amino acid sequences of SEQ ID NO: 10 and SEQ ID NO: 9 and contains CDR-H1, CDR-H2, CDR-H3, CDR-L1, CDR-L2, and CDR-L3 having the sequences of SEQ ID NOs: 12, 13, 14, 15, 16, and 17 respectively. In some embodiments, a Fab-Fc fusion consists of SEQ ID NO: 10 and SEQ ID NO: 9.

In some embodiments, a Fab-Fc fusion comprises amino acid sequences at least 70%, at least 80%, at least 90%, at least 95%, at least 96%, at least 97%, at least 98%, or at least 99% identical to the amino acid sequences of SEQ ID NO: 19 and SEQ ID NO: 18 and contains CDR-H1, CDR-H2, CDR-H3, CDR-L1, CDR-L2, and CDR-L3 having the sequences of SEQ ID NOs: 21, 22, 23, 24, 25, and 16, respectively. In some embodiments, a Fab-Fc fusion consists of SEQ ID NO: 19 and SEQ ID NO: 18.

In some embodiments, a Fab-Fc fusion comprises amino acid sequences at least 70%, at least 80%, at least 90%, at least 95%, at least 96%, at least 97%, at least 98%, or at least 99% identical to the amino acid sequences of SEQ ID NO: 102 and SEQ ID NO: 103. In some embodiments, a Fab-Fc fusion consists of SEQ ID NO: 102 and SEQ ID NO: 103.

In some embodiments, a Fab-Fc fusion comprises amino acid sequences at least 70%, at least 80%, at least 90%, at least 95%, at least 96%, at least 97%, at least 98%, or at least 99% identical to the amino acid sequences of SEQ ID NO: 104 and SEQ ID NO: 105 and contains CDR sequences of SEQ ID NOs. 102 and 103. In some embodiments, a Fab-Fc fusion consists of SEQ ID NO: 104 and SEQ ID NO: 105.

In some embodiments, a Fab-Fc fusion comprises amino acid sequences at least 70%, at least 80%, at least 90%, at least 95%, at least 96%, at least 97%, at least 98%, or at least 99% identical to the amino acid sequences of SEQ ID NO: 110 and SEQ ID NO: 111 and contains CDR-H1, CDR-H2, CDR-H3, CDR-L1, CDR-L2, and CDR-L3 having the sequences of SEQ ID NOs: 114, 115, 116, 117, 118, and 119, respectively. In some embodiments, a Fab-Fc fusion consists of SEQ ID NO: 110 and SEQ ID NO: 111.

In some embodiments, a Fab-Fc fusion comprises amino acid sequences at least 70%, at least 80%, at least 90%, at least 95%, at least 96%, at least 97%, at least 98%, or at least 99% identical to the amino acid sequences of SEQ ID NO: 122 and SEQ ID NO: 123 and contains CDR-H1, CDR-H2, CDR-H3, CDR-L1, CDR-L2, and CDR-L3 having the sequences of SEQ ID NOs: 126, 127, 128, 129, 130, and 131, respectively. In some embodiments, a Fab-Fc fusion consists of SEQ ID NO: 122 and SEQ ID NO: 123.

In some embodiments, a Fab-Fc fusion comprises amino acid sequences at least 70%, at least 80%, at least 90%, at least 95%, at least 96%, at least 97%, at least 98%, or at least 99% identical to the amino acid sequences of SEQ ID NO: 132 and SEQ ID NO: 133 and contains CDR-H1, CDR-H2, CDR-H3, CDR-L1, CDR-L2, and CDR-L3 having the sequences of SEQ ID NOs: 134, 135, 136, 137, 138, and 139, respectively. In some embodiments, a Fab-Fc fusion consists of SEQ ID NO: 132 and SEQ ID NO: 133.

In some embodiments, a Fab-Fc fusion comprises amino acid sequences at least 70%, at least 80%, at least 90%, at least 95%, at least 96%, at least 97%, at least 98%, or at least 99% identical to the amino acid sequences of SEQ ID NO: 143 and SEQ ID NO: 144 and contains CDR-H1, CDR-H2, CDR-H3, CDR-L1, CDR-L2, and CDR-L3 having the sequences of SEQ ID NOs: 147, 148, 149, 150, 151, and 152, respectively. In some embodiments, a Fab-Fc fusion consists of SEQ ID NO: 143 and SEQ ID NO: 144.

ScFabs can be made by forming a fusion protein comprising the heavy chain and light chain of any of the described Fabs using methods known in the art.

Illustrative Proteins Comprising scFvs that Specifically Bind TfR

Exemplary scFvs that specifically binds to a TfR include the heavy chain variable region of SEQ ID NO: 10, 19, 102, 104, 110, 122, 132, or 143 and the light chain variable region of SEQ ID NO: 9, 18, 103, 105, 111, 123, 133, or 144. Unless otherwise apparent from context, reference to a scFv that specifically binds to a TfR should be understood as referring to any of mouse, chimeric, veneered, humanized, and modified forms.

In some embodiments, a scFv that specifically binds to a TfR comprises the heavy chain variable region of SEQ ID NO: 10 and the light chain variable region of SEQ ID NO: 9. In some embodiments, a scFv that specifically binds to a TfR comprises the heavy chain variable region of SEQ ID NO: 19 and the light chain variable region of SEQ ID NO: 18. In some embodiments, a scFv that specifically binds to a TfR comprises the heavy chain variable region of SEQ ID NO: 102 and the light chain variable region of SEQ ID NO: 103. In some embodiments, a scFv that specifically binds to a TfR comprises the heavy chain variable region of SEQ ID NO: 104 and the light chain variable region of SEQ ID NO: 105. In some embodiments, a scFv that specifically binds to a TfR comprises the heavy chain variable region of SEQ ID NO: 110 and the light chain variable region of SEQ ID NO: 111. In some embodiments, a scFv that specifically binds to a TfR comprises the heavy chain variable region of SEQ ID NO: 122 and the light chain variable region of SEQ ID NO: 123. In some embodiments, a scFv that specifically binds to a TfR comprises the heavy chain variable region of SEQ ID NO: 132 and the light chain variable region of SEQ ID NO: 133. In some embodiments, a scFv that specifically binds to a TfR comprises the heavy chain variable region of SEQ ID NO: 143 and the light chain variable region of SEQ ID NO: 144.

In some embodiments, a scFv that specifically binds to a TfR comprises SEQ ID NO: 106. In some embodiments, a scFv that specifically binds to a TfR comprises SEQ ID NO: 107. In some embodiments, a scFv that specifically binds to a TfR comprises SEQ ID NO: 171. In some embodiments, a scFv that specifically binds to a TfR comprises SEQ ID NO: 153. In some embodiments, a scFv that specifically binds to a TfR comprises SEQ ID NO: 160. In some embodiments, a scFv that specifically binds to a TfR comprises SEQ ID NOs: 112 and 113. In some embodiments, a scFv that specifically binds to a TfR comprises SEQ ID NOs: 124 and 125. In some embodiments, a scFv that specifically binds to a TfR comprises SEQ ID NOs: 145 and 146.

In some embodiments, a scFv that specifically binds to a TfR consists of SEQ ID NO: 106. In some embodiments, a scFv that specifically binds to a TfR consists of SEQ ID NO: 107. In some embodiments, a scFv that specifically binds to a TfR consists of SEQ ID NO: 171. In some embodiments, a scFv that specifically binds to a TfR consists of SEQ ID NO: 153. In some embodiments, a scFv that specifically binds to a TfR consists of SEQ ID NO: 160.

In some embodiments, a scFv that specifically binds to a TfR comprises the CDR sequences of SEQ ID NOs: 10 and 9, 19 and 18, 102 and 103, 104 and 105, 110 and 111, 122 and 123, 132 and 133; or 143 and 144.

In some embodiments, a scFv that specifically binds to a TfR comprises CDR-H1, CDR-H2, CDR-H3, CDR-L1, CDR-L2, and CDR-L3 having the sequences of SEQ ID NOs: 12, 13, 14, 15, 16, and 17, respectively. In some embodiments, a scFv that specifically binds to a TfR comprises CDR-H1, CDR-H2, CDR-H3, CDR-L1, CDR-L2, and CDR-L3 having the sequences of SEQ ID NOs: 21, 22, 23, 24, 25, and 26, respectively. In some embodiments, a scFv that specifically binds to a TfR comprises CDR-H1, CDR-H2, CDR-H3, CDR-L1, CDR-L2, and CDR-L3 having the sequences of SEQ ID NOs: 114, 115, 116, 117, 118, and 119, respectively. In some embodiments, a scFv that specifically binds to a TfR comprises CDR-H1, CDR-H2, CDR-H3, CDR-L1, CDR-L2, and CDR-L3 having the sequences of SEQ ID NOs: 126, 127, 128, 129, 130, and 131, respectively. In some embodiments, a scFv that specifically binds to a TfR comprises CDR-H1, CDR-H2, CDR-H3, CDR-L1, CDR-L2, and CDR-L3 having the sequences of SEQ ID NOs: 134, 135, 136, 137, 138, and 139, respectively. In some embodiments, a scFv that specifically binds to a TfR comprises CDR-H1, CDR-H2, CDR-H3, CDR-L1, CDR-L2, and CDR-L3 having the sequences of SEQ ID NOs: 154, 155, 156, 157, 158, and 159 respectively. In some embodiments, a scFv that specifically binds to a TfR comprises CDR-H1, CDR-H2, CDR-H3, CDR-L1, CDR-L2, and CDR-L3 having the sequences of SEQ ID NOs: 161, 162, 163, 164, 165, and 166, respectively.

In some embodiments, a scFv that specifically binds to a TfR comprises an amino acid sequence that is at least 70%, at least 80%, at least 90%, at least 95%, at least 96%, at least 97%, at least 98%, or at least 99% identical to the amino acid sequence of SEQ ID NO: 106, 107, or 171 and contains the CDR sequences of the SEQ ID NOs: 102 and 103. In some embodiments, a scFv that specifically binds to a TfR comprises an amino acid sequence that is at least 70%, at least 80%, at least 90%, at least 95%, at least 96%, at least 97%, at least 98%, or at least 99% identical to the amino acid sequence of SEQ ID NO: 153 and contains CDR-H1, CDR-H2, CDR-H3, CDR-L1, CDR-L2, and CDR-L3 having the sequences of SEQ ID NOs: 154, 155, 156, 157, 158, and 159, respectively. In some embodiments, a scFv that specifically binds to a TfR comprises an amino acid sequence that is at least 70%, at least 80%, at least 90%, at least 95%, at least 96%, at least 97%, at least 98%, or at least 99% identical to the amino acid sequence of SEQ ID NO: 160 and contains CDR-H1, CDR-H2, CDR-H3, CDR-L1, CDR-L2, and CDR-L3 having the sequences of SEQ ID NOs: 161, 162, 163, 164, 165, and 166, respectively.

In some embodiments, a scFv that specifically binds to a TfR comprises an amino acid sequence that is at least 70%, at least 80%, at least 90%, at least 95%, at least 96%, at least 97%, at least 98%, or at least 99% identical to the amino acid sequence of SEQ ID NO: 112 and an amino acid sequence that is at least 70%, at least 80%, at least 90%, at least 95%, at least 96%, at least 97%, at least 98%, or at least 99% identical to the amino acid sequence of SEQ ID NO: 113, and contains CDR-H1, CDR-H2, CDR-H3, CDR-L1, CDR-L2, and CDR-L3 having the sequences of SEQ ID NOs: 114, 115, 116, 117, 118, and 119 (i.e., the CDR sequences of SEQ ID NOs: 110 and 111), respectively.

In some embodiments, a scFv that specifically binds to a TfR comprises an amino acid sequence that is at least 70%, at least 80%, at least 90%, at least 95%, at least 96%, at least 97%, at least 98%, or at least 99% identical to the amino acid sequence of SEQ ID NO: 124 and an amino acid sequence that is at least 70%, at least 80%, at least 90%, at least 95%, at least 96%, at least 97%, at least 98%, or at least 99% identical to the amino acid sequence of SEQ ID NO: 125, and contains CDR-H1, CDR-H2, CDR-H3, CDR-L1, CDR-L2, and CDR-L3 having the sequences of SEQ ID NOs: 126, 127, 128, 129, 130, and 131 (i.e., the CDR sequences of SEQ ID NOs: 120 and 121), respectively.

In some embodiments, a scFv that specifically binds to a TfR comprises an amino acid sequence that is at least 70%, at least 80%, at least 90%, at least 95%, at least 96%, at least 97%, at least 98%, or at least 99% identical to the amino acid sequence of SEQ ID NO: 145 and an amino acid sequence that is at least 70%, at least 80%, at least 90%, at least 95%, at least 96%, at least 97%, at least 98%, or at least 99% identical to the amino acid sequence of SEQ ID NO: 146, and contains CDR-H1, CDR-H2, CDR-H3, CDR-L1, CDR-L2, and CDR-L3 having the sequences of SEQ ID NOs: 147, 148, 149, 150, 151, and 152 (i.e., the CDR sequences of SEQ ID NOs: 140 and 142), respectively.

In some embodiments, a scFv that specifically binds to a TfR comprises light chain and heavy chain variably regions that differ from the variable regions of SEQ ID NOs: 9 and 10, 18 and 19, 102 and 103, 104 and 105, 110 and 111, 122 and 123, 132 and 133; or 143 and 144 by a small number of functionally inconsequential amino acid substitutions (e.g., conservative substitutions), deletions, or insertions.

Illustrative Proteins Comprising Antibodies that Specifically Bind TfR

Exemplary antibodies that specifically binds to a TfR include the heavy chain variable region of SEQ ID NO: 10, 19, 102, 104, 110, 122, 132, or 143 and the light chain variable region of SEQ ID NO: 9, 18, 103, 105, 111, 123, 133, or 144. Unless otherwise apparent from context, reference to an antibody that specifically binds to a TfR should be understood as referring to any of mouse, chimeric, veneered, humanized, and modified forms.

In some embodiments, an antibody that specifically binds to a TfR comprises the heavy chain variable region of SEQ ID NO: 10 and the light chain variable region of SEQ ID NO: 9. In some embodiments, an antibody that specifically binds to a TfR comprises the heavy chain variable region of SEQ ID NO: 19 and the light chain variable region of SEQ ID NO: 18. In some embodiments, an antibody that specifically binds to a TfR comprises the heavy chain variable region of SEQ ID NO: 102 and the light chain variable region of SEQ ID NO: 103. In some embodiments, an antibody that specifically binds to a TfR comprises the heavy chain variable region of SEQ ID NO: 104 and the light chain variable region of SEQ ID NO: 105. In some embodiments, an antibody that specifically binds to a TfR comprises the heavy chain variable region of SEQ ID NO: 110 and the light chain variable region of SEQ ID NO: 111. In some embodiments, an antibody that specifically binds to a TfR comprises the heavy chain variable region of SEQ ID NO: 122 and the light chain variable region of SEQ ID NO: 123. In some embodiments, an antibody that specifically binds to a TfR comprises the heavy chain variable region of SEQ ID NO: 132 and the light chain variable region of SEQ ID NO: 133. In some embodiments, an antibody that specifically binds to a TfR comprises the heavy chain variable region of SEQ ID NO: 143 and the light chain variable region of SEQ ID NO: 144.

In some embodiments, an antibody that specifically binds to a TfR comprises the heavy chain CH1 and variable regions of SEQ ID NO: 10 and a light chain comprising SEQ ID NO: 9. In some embodiments, an antibody that specifically binds to a TfR comprises the heavy chain CH1 and variable regions of SEQ ID NO: 19 and a light chain comprising SEQ ID NO: 18. In some embodiments, an antibody that specifically binds to a TfR comprises a heavy chain comprising SEQ ID NO: 102 and a light chain comprising SEQ ID NO: 103. In some embodiments, an antibody that specifically binds to a TfR comprises a heavy chain comprising SEQ ID NO: 104 and a light chain comprising SEQ ID NO: 105. In some embodiments, an antibody that specifically binds to a TfR comprises a heavy chain comprising SEQ ID NO: 110 and a light chain comprising SEQ ID NO: 111. In some embodiments, an antibody that specifically binds to a TfR comprises a heavy chain comprising SEQ ID NO: 122 and a light chain comprising SEQ ID NO: 123. In some embodiments, an antibody that specifically binds to a TfR comprises a heavy chain comprising SEQ ID NO: 132 and a light chain comprising SEQ ID NO: 133. In some embodiments, an antibody that specifically binds to a TfR comprises a heavy chain comprising SEQ ID NO: 143 and a light chain comprising SEQ ID NO: 144.

In some embodiments, an antibody that specifically binds to a TfR comprises SEQ ID NO: 108 and SEQ ID NO: 109. In some embodiments, an antibody that specifically binds to a TfR comprises SEQ ID NO: 120 and SEQ ID NO: 121. In some embodiments, an antibody that specifically binds to a TfR comprises SEQ ID NO: 9, SEQ ID NO: 10, and SEQ ID NO: 11. In some embodiments, an antibody that specifically binds to a TfR comprises SEQ ID NO: 18 and SEQ ID NO: 19.

In some embodiments, an antibody that specifically binds to a TfR comprises the CDR sequences of SEQ ID NOs: 10 and 9, 19 and 18, 102 and 103, 104 and 105, 110 and 111, 122 and 123, 132 and 133; or 143 and 144.

In some embodiments, an antibody that specifically binds to a TfR comprises CDR-H1, CDR-H2, CDR-H3, CDR-L1, CDR-L2, and CDR-L3 having the sequences of SEQ ID NOs: 12, 13, 14, 15, 16, and 17, respectively. In some embodiments, an antibody that specifically binds to a TfR comprises CDR-H1, CDR-H2, CDR-H3, CDR-L1, CDR-L2, and CDR-L3 having the sequences of SEQ ID NOs: 21, 22, 23, 24, 25, and 26, respectively. In some embodiments, an antibody that specifically binds to a TfR comprises CDR-H1, CDR-H2, CDR-H3, CDR-L1, CDR-L2, and CDR-L3 having the sequences of SEQ ID NOs: 114, 115, 116, 117, 118, and 119, respectively. In some embodiments, an antibody that specifically binds to a TfR comprises CDR-H1, CDR-H2, CDR-H3, CDR-L1, CDR-L2, and CDR-L3 having the sequences of SEQ ID NOs: 126, 127, 128, 129, 130, and 131, respectively. In some embodiments, an antibody that specifically binds to a TfR comprises CDR-H1, CDR-H2, CDR-H3, CDR-L1, CDR-L2, and CDR-L3 having the sequences of SEQ ID NOs: 134, 135, 136, 137, 138, and 139, respectively. In some embodiments, an antibody that specifically binds to a TfR comprises CDR-H1, CDR-H2, CDR-H3, CDR-L1, CDR-L2, and CDR-L3 having the sequences of SEQ ID NOs: 154, 155, 156, 157, 158, and 159 respectively. In some embodiments, an antibody that specifically binds to a TfR comprises CDR-H1, CDR-H2, CDR-H3, CDR-L1, CDR-L2, and CDR-L3 having the sequences of SEQ ID NOs: 161, 162, 163, 164, 165, and 166, respectively.

In some embodiments, an antibody that specifically binds to a TfR comprises a heavy chain containing an amino acid sequence that is at least 70%, at least 80%, at least 90%, at least 95%, at least 96%, at least 97%, at least 98%, or at least 99% identical to the VH and CH1 region amino acid sequence of SEQ ID NOs: 10, 19, 102, 104, 110, 122, 132, or 143; a light chain containing an amino acid sequence that is at least 70%, at least 80%, at least 90%, at least 95%, at least 96%, at least 97%, at least 98%, or at least 99% identical to the amino acid sequence of SEQ ID NOs: 9, 18, 103, 105, 111, 123, 133, or 144, and contains the CDR sequences of the SEQ ID NOs: 10 and 9, 19 and 18, 102 and 103, 104 and 105, 110 and 111, 122 and 123, 132 and 133; or 143 and 144, respectively.

In some embodiments, an antibody that specifically binds to a TfR comprises a first heavy chain containing an amino acid sequence that is at least 70%, at least 80%, at least 90%, at least 95%, at least 96%, at least 97%, at least 98%, or at least 99% identical to SEQ ID NO: 10, a second heavy chain containing an amino acid sequence that is at least 70%, at least 80%, at least 90%, at least 95%, at least 96%, at least 97%, at least 98%, or at least 99% identical to SEQ ID NOs: 11, a light chain containing an amino acid sequence that is at least 70%, at least 80%, at least 90%, at least 95%, at least 96%, at least 97%, at least 98%, or at least 99% identical to the amino acid sequence of SEQ ID NOs: 9, and contains the CDR sequences of the SEQ ID NOs: 10 and 9.

In some embodiments, an antibody that specifically binds to a TfR comprises a heavy chain containing an amino acid sequence that is at least 70%, at least 80%, at least 90%, at least 95%, at least 96%, at least 97%, at least 98%, or at least 99% identical to SEQ ID NO: 19, a light chain containing an amino acid sequence that is at least 70%, at least 80%, at least 90%, at least 95%, at least 96%, at least 97%, at least 98%, or at least 99% identical to the amino acid sequence of SEQ ID NOs: 18, and contains the CDR sequences of the SEQ ID NOS: 19 and 18.

In some embodiments, an antibody that specifically binds to a TfR comprises a heavy chain containing an amino acid sequence that is at least 70%, at least 80%, at least 90%, at least 95%, at least 96%, at least 97%, at least 98%, or at least 99% identical to SEQ ID NO: 108, a light chain containing an amino acid sequence that is at least 70%, at least 80%, at least 90%, at least 95%, at least 96%, at least 97%, at least 98%, or at least 99% identical to the amino acid sequence of SEQ ID NOs: 109, and contains the CDR sequences of the SEQ ID NOs: 108 and 109 (i.e., SEQ ID NOs. 114-119).

In some embodiments, an antibody that specifically binds to a TfR comprises a heavy chain containing an amino acid sequence that is at least 70%, at least 80%, at least 90%, at least 95%, at least 96%, at least 97%, at least 98%, or at least 99% identical to SEQ ID NO: 120, a light chain containing an amino acid sequence that is at least 70%, at least 80%, at least 90%, at least 95%, at least 96%, at least 97%, at least 98%, or at least 99% identical to the amino acid sequence of SEQ ID NOs: 121, and contains the CDR sequences of the SEQ ID NOs: 120 and 121 (i.e., SEQ ID NOs. 126-131).

Any of the described antibodies that specifically binds to a TfR can have one or more modifications to increase serum stability, modulate effector function, influence glycosylation, reduce immunogenicity in humans, facilitate heterodimerization, and/or facilitate conjugation of the oligonucleotide.

Any of the described antibodies that specifically binds to a TfR can have Fc polypeptides comprising amino acid sequences selected from the group consisting of: SEQ ID NOs: 27-34, 79-90, and 92-98. The Fc polypeptides may be modified to increase serum stability, modulate effector function, influence glycosylation, reduce immunogenicity in humans, facilitate heterodimerization, and/or facilitate conjugation of the oligonucleotide.

In some embodiments, an antibody that specifically binds to a TfR comprises light chain and heavy chain variably regions that differ from the variable regions of SEQ ID NOs: 9 and 10, 18 and 19, 102 and 103, 104 and 105, 110 and 111, 122 and 123, 132 and 133; or 143 and 144 by a small number of functionally inconsequential amino acid substitutions (e.g., conservative substitutions), deletions, or insertions.

Additional exemplary anti-TfR antibody antigen binding domains include, but are not limited to: a 17H10 anti-TfR Fab or scFv; a 17H10.1 anti-TfR Fab or scFv; a JC-141 anti-TfR antibody; a JC-141 anti-TfR Fab; a JC-141 anti-TfR scFv; an anti-TfR antibody, Fab, scFab, Fv fragment, or scFv having the heavy chain and light chain CDR1, CDR2, and CDR3 sequences of the JR-141 antibody (WO2016208695); a JC-171 anti-TfR antibody; a JC-171 anti-TfR Fab; a JC-171 anti-TfR scFv; an anti-TfR antibody, Fab, scFab, Fv fragment, or scFv having the heavy chain and light chain CDR1, CDR2, and CDR3 sequences of the JR-171 antibody (WO2018124121); a “Brain shuttle” (BS) anti-TfR Fab; an anti-TfR antibody, Fab, scFab, Fv fragment, or scFv having the heavy chain and light chain CDR1, CDR2, and CDR3 sequences of the BS anti-TfR Fab (WO2018210898, WO2015101588, and WO2014033074); a 13E4v2ii anti-TfR antibody; a 13E4v2ii anti-TfR Fab; a 13E4v2ii anti-TfR scFv; an anti-TfR antibody, Fab, scFab, Fv fragment, or scFv having the heavy chain and light chain CDR1, CDR2, and CDR3 sequences of the 13E4v2ii antibody (WO2020132584); a TfR12 anti-TfR scFv; an anti-TfR antibody, Fab, scFab, Fv fragment, or scFv having the heavy chain and light chain CDR1, CDR2, and CDR3 sequences of the TfR12 anti-TfR scFv (WO2021/205358); a TfR13 anti-TfR scFv; or an anti-TfR antibody, Fab, scFab, Fv fragment, or scFv having the heavy chain and light chain CDR1, CDR2, and CDR3 sequences of the Tfr13 anti-TfR scFv (WO2021/205358) (Sequences shown in Table 1).

Additional anti-TfR antibodies are described in WO2021/205358, and the anti-TfR antibody antigen binding domains of the invention can include any antibody antigen binding domain with the CDRs or variable regions of any one of TfR1, TfR2, TfR3, TfR4, TfR5, TfR6, TfR7, TfR8, TfR9, TfR10, TfR11, TfR12, TfR13, TfR14, TfR15, TfR16, TfR17, TfR18, TfR19, TfR20, TfR21, TfR22, TfR23, TfR24, TfR25, TfR26, TfR27, TfR28, TfR29, TfR30, TfR31, TfR32, TfR33, TfR34, TfR35, TfR36, TfR37, and TfR38 described therein.

TABLE 1
Exemplary TfR-binding regions comprising antibody antigen binding domains.

| | |
|---|---|
| 17H10 | Heavy chain: |
| anti-TfR Fab | EVQLVESGGGLVQPGGSLRLSCAVSGFSLTTYGVDWVRQAPGKGLEWVGIIWGDGTTHYHSAL |
| | ISRFTISKDNSKNTVYLQMNSLRAEDTAVYYCAKPGGASWGQGTLVTVSSASTKGPSVFPLAP |
| | SSKSTSGGTAALGCLVKDYFPEPVTVSWNSGALTSGVHTFPAVLQSSGLYSLSSVVTVPSSSL |
| | GTQTYICNVNHKPSNTKVDKKVEPKSCDKTH (SEQ ID NO: 102) |
| | Light chain: |
| | DIQMTQSPSSLSASVGDRVTITCKASQLVGTNVAWYQQKPGKSPKALFYSASYRNQGVPSRES |
| | GSGSGTDFTLTISSLQPEDFATYFCQHYNSYPFTFGQGTKVEIKRTVAAPSVFIFPPSDEQLK |
| | SGTASVVCLLNNFYPREAKVQWKVDNALQSGNSQESVTEQDSKQSTYSLSSTLTLSKADYEKH |
| | KVYACEVTHQGLSSPVTKSENRGEC (SEQ ID NO: 103) |
| 17H10.1 | Heavy chain: |
| anti-TfR Fab | EVQLVESGGGLVQPGGSLRLSCAVSGFSLTTYGVDWVRQAPGKGLEWVGIIWGDGTTHYHSAL |
| | ISRFTISKDNSKNTVYLQMNSLRAEDTAVYYCAKPGGASWGQGTLVTVSSASTKGPSVEPLAP |
| | SSKSTSGGTAALGCLVKDYFPEPVTVSWNSGALTSGVHTFPAVLQSSGLYSLSSVVTVPSSSL |
| | GTQTYICNVNHKPSNTKVDKKVEPKSCDKTH (SEQ ID NO: 104) |
| | Light chain: |
| | DIQMTQSPSSLSASVGDRVTITCKASQFVGTAVAWYQQKPGKSPKALFYSASYRNEGVPSRFS |
| | GSGSGTDETLTISSLQPEDFATYFCQHYNSYPFTFGQGTKVEIKRTVAAPSVFIFPPSDEQLK |
| | SGTASVVCLLNNFYPREAKVQWKVDNALQSGNSQESVTEQDSKQSTYSLSSTLTLSKADYEKH |
| | KVYACEVTHQGLSSPVTKSENRGEC (SEQ ID NO: 105) |
| 17H10 | EVQLVESGGGLVQPGGSLRLSCAVSGFSLTTYGVDWVRQAPGKGLEWVGIIWGDGTTHYHSAL |
| anti-TfR scFv | ISRFTISKDNSKNTVYLQMNSLRAEDTAVYYCAKPGGASWGQGTLVTVSSGGGGSGGGGSGGG |
| | GSDIQMTQSPSSLSASVGDRVTITCKASQLVGTNVAWYQQKPGKSPKALFYSASYRNQGVPSR |
| | FSGSGSGTDFTLTISSLQPEDFATYFCQHYNSYPFTFGQGTKVEIKEPKSS |
| | (SEQ ID NO: 106) |
| 17H10.1 | EVQLVESGGGLVQPGGSLRLSCAVSGFSLTTYGVDWVRQAPGKGLEWVGIIWGDGTTHYHSAL |
| anti-TfR scFv | ISRFTISKDNSKNTVYLQMNSLRAEDTAVYYCAKPGGASWGQGTLVTVSSGGGGSGGGGSGGG |
| | GSDIQMTQSPSSLSASVGDRVTITCKASQFVGTAVAWYQQKPGKSPKALFYSASYRNEGVPSR |
| | FSGSGSGTDFTLTISSLQPEDFATYFCQHYNSYPFTFGQGTKVEIKEPKSS |
| | (SEQ ID NO: 107) |
| 17H10 | EVQLVESGGGLVQPGGSLRLSCAVSGFSLTTYGVDWVRQAPGKGLEWVGIIWGDGTTHYHSAL |
| anti-TfR scFv | ISRFTISKDNSKNTVYLQMNSLRAEDTAVYYCAKPGGASWGQGTLVTVSSGGGGSGGGGSGGG |
| alternate | GSDIQMTQSPSSLSASVGDRVTITCKASQNVGTNVAWYQQKPGKSPKALFYSASYRNAGVPSR |
| | FSGSGSGTDFTLTISSLQPEDFATYFCQHYNSYPFTFGQGTKVEIK (SEQ ID NO: 171) |
| JC-141 | Heavy chain: |
| anti-TfR | EVQLVQSGAEVKKPGESLKISCKGSGYSFTNYWLGWVRQMPGKGLEWMGDIYPGGDYPTYSEK |
| antibody | FKVQVTISADKSISTAYLQWSSLKASDTAMYYCARSGNYDEVAYWGQGTLVTVSSASTKGPSV |
| | FPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSCDKTHTCPPCPAPELLGGPSVFLFPPKPKQTL |
| | MISRTPEVTCVVVDVSHEDPEVKFNWYVDGVEVHNAKTKPREEQYNSTYRVVSVLTVLHQDWL |
| | NGKEYKCKVSNKALPAPIEKTISKAKGQPREPQVYTLPPSRDELTKNQVSLTCLVKGFYPSDI |
| | AVEWESNGQPENNYKTTPPVLDSDGSFFLYSKLTVDKSRWQQGNVFSCSVMHEALHNHYTQKS |
| | LSLSPGK (SEQ ID NO: 108) |
| | Light chain: |
| | DIVMTQTPLSLSVTPGQPASISCRSSQSLVHSNGNTYLHWYLQKPGQSPQLLIYKVSNRFSGV |
| | PDRFSGSGSGTDFTLKISRVEAEDVGVYYCSQSTHVPWTFGQGTKVEIKRTVAAPSVFIFPPS |
| | DEQLKSGTASVVCLLNNFYPREAKVQWKVDNALQSGNSQESVTEQDSKDSTYSLSSTLTLSKA |
| | DYEKHKVYACEVTHQGLSSPVTKSENRGEC (SEQ ID NO: 109) |
| JC-141 | Heavy chain: |
| anti-TfR Fab | EVQLVQSGAEVKKPGESLKISCKGSGYSFTNYWLGWVRQMPGKGLEWMGDIYPGGDYPTYSEK |
| | FKVQVTISADKSISTAYLQWSSLKASDTAMYYCARSGNYDEVAYWGQGTLVTVSSASTKGPSV |
| | FPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSC (SEQ ID NO: 110) |
| | Light chain: |
| | DIVMTQTPLSLSVTPGQPASISCRSSQSLVHSNGNTYLHWYLQKPGQSPQLLIYKVSNRFSGV |
| | PDRFSGSGSGTDFTLKISRVEAEDVGVYYCSQSTHVPWTFGQGTKVEIKRTVAAPSVFIFPPS |
| | DEQLKSGTASVVCLLNNFYPREAKVQWKVDNALQSGNSQESVTEQDSKDSTYSLSSTLTLSKA |
| | DYEKHKVYACEVTHQGLSSPVTKSENRGEC (SEQ ID NO: 111) |
| JC-141 | Heavy chain: |
| anti-TfR | EVQLVQSGAEVKKPGESLKISCKGSGYSFTNYWLGWVRQMPGKGLEWMGDIYPGGDYPTYSEK |
| scFv | FKVQVTISADKSISTAYLQWSSLKASDTAMYYCARSGNYDEVAYWGQGTLVTVSS |
| sequences | (SEQ ID NO: 112) |
| | Light chain: |
| | DIVMTQTPLSLSVTPGQPASISCRSSQSLVHSNGNTYLHWYLQKPGQSPQLLIYKVSNRFSGV |
| | PDRFSGSGSGTDFTLKISRVEAEDVGVYYCSQSTHVPWTFGQGTKVEIK (SEQ ID NO: |
| | 113) |
| JC-141 | Heavy chain: |
| anti-TfR | CDR1: GYSETNYWLG (SEQ ID NO: 114) |
| CDR | CDR2: DIYPGGDYPTYSEKFKV (SEQ ID NO: 115) |
| sequences | CDR3: SGNYDEVAY (SEQ ID NO: 116) |
| | Light chain: |
| | CDR1: RSSQSLVHSNGNTYLH (SEQ ID NO: 117) |
| | CDR2: KVSNRFS (SEQ ID NO: 118) |
| | CDR3: SQSTHVPWT (SEQ ID NO: 119) |
| JC-171 | Heavy Chain: |
| anti-TfR | EVQLVQSGAEVKKPGESLKISCKGSGYSFMNYWLGWVRQMPGKGLEWMGDIYPGGDYPTYSEK |
| antibody | FKVQVTISADKSISTAYLQLSSLKASDTAMYYCARSGNYDEVAYWGQGTLVTVSSASTKGPSV |
| | FPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSCDKTHTCPPCPAPELLGGPSVFLFPPKPKQTL |
| | MISRTPEVTCVVVDVSHEDPEVKFNWYVDGVEVHNAKTKPREEQYNSTYRVVSVLTVLHQDWL |
| | NGKEYKCKVSNKALPAPIEKTISKAKGQPREPQVYTLPPSRDELTKNQVSLTCLVKGFYPSDI |
| | AVEWESNGQPENNYKTTPPVLDSDGSFFLYSKLTVDKSRWQQGNVFSCSVMHEALHNHYTQKS |
| | LSLSPGK (SEQ ID NO: 120) |
| | Light chain: |
| | DIVMTQTPLSLSVTPGQPASISCRSSQSLVHSNGNTYLHWYLQKPGQSPQLLIYKVSNRFSGV |
| | PDRFSGSGSGTDFTLKISRVEAEDVGVYYCSQSTHVPWTFGQGTKVEIKRTVAAPSVFIFPPS |
| | DEQLKSGTASVVCLLNNFYPREAKVQWKVDNALQSGNSQESVTEQDSKDSTYSLSSTLTLSKA |
| | DYEKHKVYACEVTHQGLSSPVTKSENRGEC (SEQ ID NO: 121) |
| JC-171 | Heavy Chain: |
| anti-TfR | EVQLVQSGAEVKKPGESLKISCKGSGYSFMNYWLGWVRQMPGKGLEWMGDIYPGGDYPTYSEK |
| Fab | FKVQVTISADKSISTAYLQLSSLKASDTAMYYCARSGNYDEVAYWGQGTLVTVSSASTKGPSV |
| | FPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSC (SEQ ID NO: 122) |
| | Light chain: |
| | DIVMTQTPLSLSVTPGQPASISCRSSQSLVHSNGNTYLHWYLQKPGQSPQLLIYKVSNRFSGV |
| | PDRFSGSGSGTDFTLKISRVEAEDVGVYYCSQSTHVPWTFGQGTKVEIKRTVAAPSVFIFPPS |
| | DEQLKSGTASVVCLLNNFYPREAKVQWKVDNALQSGNSQESVTEQDSKDSTYSLSSTLTLSKA |
| | DYEKHKVYACEVTHQGLSSPVTKSENRGEC (SEQ ID NO: 123) |
| JC-171 | Heavy Chain: |
| anti-TfR | EVQLVQSGAEVKKPGESLKISCKGSGYSFMNYWLGWVRQMPGKGLEWMGDIYPGGDYPTYSEK |
| scFv | FKVQVTISADKSISTAYLQLSSLKASDTAMYYCARSGNYDEVAYWGQGTLVTVSS |
| sequences | (SEQ ID NO: 124) |
| | Light chain: |
| | DIVMTQTPLSLSVTPGQPASISCRSSQSLVHSNGNTYLHWYLQKPGQSPQLLIYKVSNRFSGV |
| | PDRFSGSGSGTDFTLKISRVEAEDVGVYYCSQSTHVPWTFGQGTKVEIK (SEQ ID NO: |
| | 125) |
| JC-171 | Heavy chain: |
| anti-TfR | CDR1: GYSEMNYWLG (SEQ ID NO: 126) |
| CDR | CDR2: DIYPGGDYPTYSEKFKV (SEQ ID NO: 127) |
| sequences | CDR3: SGNYDEVAY (SEQ ID NO: 128) |
| | Light chain: |
| | CDR1: RSSQSLVHSNGNTYLH (SEQ ID NO: 129) |
| | CDR2: KVSNRFS (SEQ ID NO: 130) |
| | CDR3: SQSTHVPWT (SEQ ID NO: 131) |
| BS anti-TfR | Heavy Chain: |
| Fab | QSMQESGPGLVKPSQTLSLTCTVSGFSLSSYAMSWIRQHPGKGLEWIGYIWSGGSTDYASWAK |
| | SRVTISKTSTTVSLKLSSVTAADTAVYYCARRYGTSYPDYGDASGFDPWGQGTLVTVSSASVA |
| | APSVFIFPPSDEQLKSGTASVVCLLNNFY?REAKVQWKVDNALQSGNSQESVTEQDSKDSTYS |
| | LSSTLTLSKADYEKHKVYACEVTHQGLSSPVTKSENRGEC (SEQ ID NO: 132) |
| | Light chain: |
| | AIQLTQSPSSLSASVGDRVTITCRASQSISSYLAWYQQKPGKAPKLLIYRASTLASGVPSRES |
| | GSGSGTDFTLTISSLQPEDFATYYCQQNYASSNVDNTFGGGTKVEIKSSASTKGPSVFPLAPS |
| | SKSTSGGTAALGCLVKDYFPEPVTVSWNSGALTSGVHTFPAVLQSSGLYSLSSVVTVPSSSLG |
| | TQTYICNVNHKPSNTKVDKKVEPKSC (SEQ ID NO: 133) |
| BS anti-TfR | Heavy chain: |
| Fab CDR | CDR1: GFSLSSYAMS (SEQ ID NO: 134) |
| sequences | CDR2: YIWSGGSTDYASWAKS (SEQ ID NO: 135) |
| | CDR3: RYGTSYPDYGDASGEDP (SEQ ID NO: 136) |
| | Light chain: |
| | CDR1: RASQSISSYLA (SEQ ID NO: 137) |
| | CDR2: RASTLAS (SEQ ID NO: 138) |
| | CDR3: QQNYASSNVDNT (SEQ ID NO: 139) |
| 13E4v2ii | Heavy Chain (with knob mutation): |
| anti-TfR | QVQLVQSGAEVKKPGASVKVSCKASGYTFTNYWMHWVRQAPGQGLEWIGEINPINGRSNYAEK |
| antibody | FQGRVTLTVDTSSSTAYMELSRLRSDDTAVYYCARGTRAMHYWGQGTLVTVSSASTKGPSVFP |
| | LAPSSKSTSGGTAALGCLVKDYFPEPVTVSWNSGALTSGVHTFPAVLQSSGLYSLSSVVTVPS |
| | SSLGTQTYICNVNHKPSNTKVDKKVEPKSCDKTHTCPPCPAPEAAGGPSVFLFPPKPKDTLMI |
| | SRTPEVTCVVVDVSHEDPEVKFNWYVDGVEVHNAKTKPREEQYNSTYRVVSVLTVLHQDWLNG |
| | KEYKCKVSNKARPAPIEKTISKAKGQPREPQVYTLPPSRDELTXNQVSLWCLVKGFYPSDIAV |
| | EWESNGQPENNYTTPPVLDSDGSFFLYSKLTVDKSRWQQGNVFSCSVMHEALHNHYTQKSLS |
| | LSPGK (SEQ ID NO: 140) |
| | Heavy Chain (with hole mutation): |
| | QVQLVQSGAEVKKPGASVKVSCKASGYTFTNYWMHWVRQAPGQGLEWIGEINPINGRSNYAEK |
| | FQGRVTLTVDTSSSTAYMELSRLRSDDTAVYYCARGTRAMHYWGQGTLVTVSSASTKGPSVEP |
| | LAPSSKSTSGGTAALGCLVKDYFPEPVTVSWNSGALTSGVHTFPAVLQSSGLYSLSSVVTVPS |
| | SSLGTQTYICNVNHKPSNTKVDKKVEPKSCDKTHTCPPCPAPEAAGGPCVFLFPPKPKDTLMI |
| | SRTPEVTCVVVDVSHEDPEVKFNWYVDGVEVHNAKTKPREEQYNSTYRVVSVLTVLHQDWLNG |
| | KEYKCKVSNKARPAPIEKTISKAKGQPRE?QVYTLPPSRDELTKNQVSLSCAVKGFYPSDIAV |
| | EWESNGQPENNYKTTPPVLDSDGSFFLVSKLTVDKSRWQQGNVFSCSVMHEALHNHYTQKSLS |
| | LSPGK (SEQ ID NO: 141) |
| | Light chain: |
| | DIQMTQSPSSLSASVGDRVTITCRTSENIYNNLAWYQQKPGKSPKLLIYAATNLADGVPSRES |
| | GSGSGTDYTLTISSLQPEDFATYYCQHFWGTPLTFGGGTKVEIKRTVAAPSVFIFPPSDEQLK |
| | SGTASVVCLLNNFYPREAKVQWKVDNALQSGNSQESVTEQDSKQSTYSLSSTLTLSKADYEKH |
| | KVYACEVTHQGLSSPVTKSENRGEC (SEQ ID NO: 142) |
| 13E4v2ii | Heavy Chain: |
| anti-TfR | QVQLVQSGAEVKKPGASVKVSCKASGYTFTNYWMHWVRQAPGQGLEWIGEINPINGRSNYAEK |
| Fab | FQGRVTLTVDTSSSTAYMELSRLRSDDTAVYYCARGTRAMHYWGQGTLVTVSSASTKGPSVFP |
| | LAPSSKSTSGGTAALGCLVKDYFPEPVTVSWNSGALTSGVHTFPAVLQSSGLYSLSSVVTVPS |
| | SSLGTQTYICNVNHKPSNTKVDKKVEPKSC (SEQ ID NO: 143) |
| | Light chain: |
| | DIQMTQSPSSLSASVGDRVTITCRTSENIYNNLAWYQQKPGKSPKLLIYAATNLADGVPSRES |
| | GSGSGTDYTLTISSLQPEDFATYYCQHFWGTPLTFGGGTKVEIKRTVAAPSVFIFPPSDEQLK |
| | SGTASVVCLLNNFYPREAKVQWKVDNALQSGNSQESVTEQDSKQSTYSLSSTLTLSKADYEKH |
| | KVYACEVTHQGLSSPVTKSENRGEC (SEQ ID NO: 144) |
| 13E4v2ii | Heavy Chain: |
| anti-TfR | QVQLVQSGAEVKKPGASVKVSCKASGYTFTNYWMHWVRQAPGQGLEWIGEINPINGRSNYAEK |
| scFv | FQGRVTLTVDTSSSTAYMELSRLRSDDTAVYYCARGTRAMHYWGQGTLVTVSS |
| sequences | (SEQ ID NO: 145) |
| | Light chain: |
| | DIQMTQSPSSLSASVGDRVTITCRTSENIYNNLAWYQQKPGKSPKLLIYAATNLADGVPSRES |
| | GSGSGTDYTLTISSLQPEDFATYYCQHFWGTPLTFGGGTKVEIK (SEQ ID NO: 146) |
| 13E4v2ii | Heavy chain: |
| anti-TfR | CDR1: GYTFTNYWMH (SEQ ID NO: 147) |
| CDR | CDR2: EINPINGRSNYAEKFQG (SEQ ID NO: 148) |
| sequences | CDR3: ARGTRAMHY (SEQ ID NO: 149) |
| | Light chain: |
| | CDR1: RTSENIYNNLA (SEQ ID NO: 150) |
| | CDR2: AATNLAD (SEQ ID NO: 151) |
| | CDR3: QHFWGTPLT (SEQ ID NO: 152) |
| Tfr12 anti- | QVQLQQWGAGLLKPSETLSLTCAVYGGSFSGYYWTWIRQPPGKGLEWIGEFIHSGSTNYNPSL |
| TfR scFv | KSRVTISVDTSKNQFSLKLSSVTAADTAVYFCARGSMDSSGFYSFDFWGQGTLVTVSSGTEGK |
| | SSGSGSESKSTSYEVTQPPSVSVSPGQTASITCSGHKLGDKFASWYQQKPGQSPVLVIYQQRK |
| | RPSGIPERFSGSNSGNTATLTISGTQAMDEADYYCQTWYSSTVIFGGGTKLTVL |
| | (SEQ ID NO: 153) |
| Tfr12 anti- | Heavy Chain: |
| TfR scFv | CDR1: GGSFSGYY (SEQ ID NO: 154) |
| CDR | CDR2: FIHSGST (SEQ ID NO: 155) |
| sequences | CDR3: ARGSMDSSGFYSFDF (SEQ ID NO: 156) |
| | Light Chain: |
| | CDR1: KLGDKF (SEQ ID NO: 157) |
| | CDR2: QDRKRP (SEQ ID NO: 158) |
| | CDR3: QTWYSSTVI (SEQ ID NO: 159) |
| Tfr13 anti- | EVQLLESGGGLVQPGGSLRLSCADSGFTFSSYAMNWVRQAPGKGLEWVSGISGSGGHTYYADS |
| TfR scFv | VKGRFTVSRDNSKNTLYLQMNSLRAEDTAVYYCAREGYDSSGYNPFDYWGQGTQVTVSSGTEG |
| | KSSGSGSESKSTSYELTQPPSVSVSPGQTASITCSGDKLGDKYASWYQQKPGQSPVLVIYQDS |
| | KRPSGIPERESGSNSGNTATLTISGTQAMDEADYYCQAWDSSTVVEGGGTKLTVL |
| | (SEQ ID NO: 160) |
| Tfr13 anti- | Heavy Chain: |
| TfR scFv | CDR1: GFTESSYAMNW (SEQ ID NO: 161) |
| CDR | CDR2: ISGSGGHT (SEQ ID NO: 162) |
| sequences | CDR3: AREGYDSSGYNPEDY (SEQ ID NO: 163) |
| | Light Chain: |
| | CDR1: KLGDKYA (SEQ ID NO: 164) |
| | CDR2: QDSKRPS (SEQ ID NO: 165) |
| | CDR3: QAWDSSTVV (SEQ ID NO: 166) |

Additional anti-TfR antibodies are known in the art and/or are available from various commercial sources. In some embodiments, the anti-TfR antibody or the TfR-binding fragment of an anti-TfR antibody binds to an apical domain of the TfR. In some embodiments, binding of the anti-TfR antibody or the TER-binding fragment of an anti-TfR antibody to the TfR does not inhibit binding of transferrin to the TfR. Exemplary anti-TfR antibodies include, but are not limited to, B3/25, RBC4, 7579, E2.3, A27.15, D65.30, D2C, ch128.1Av, ch128.1/IgG3, ch128.1/IgG1, hu128.1 (Candelaria et al. Front. Immunol. 12 (17 Mar. 2021), 2021), R17, 8D3 (Weber et al. Cell Reports 22:149-162, 2018). Exemplary anti-TfR antibodies are also described in U.S. patent publications: US2018282408A1, US2020071413A1, US20210138083A1, US20190092870A1, and US20130028891 (each of which is incorporated herein by reference).

Exemplary anti-TfR vNARs are described in WO 2022/103769.

Brain shuttles containing anti-TfR antibody antigen binding domains are described in WO 2014/033074 and WO 2015/101588 (each of which is incorporated herein by reference).

In some embodiments, the anti-TfR antibody antigen binding domain binds human TfR with an affinity of about 1 nM to about 1000 nM (e.g., about 1 nM, about 2 nM, about 5 nM, about 10 nM, about 20 nM, about 30 nM, about 40 nM, about 50 nM, about 75 nM, about 100 nM, about 150 nM, about 200 nM, about 250 nM, about 300 nM, about 400 nM, about 500 nM, about 750 nM, or about 1000 nM). In some embodiments, an anti-TfR antibody antigen binding domain binds human TfR with an affinity of about 1 nM to about 500 nM. In some embodiments, the anti-TfR antibody antigen binding domain binds human TfR with an affinity of about 1 nM to about 100 nM (e.g., about 1 nM, about 2 nM, about 5 nM, about 10 nM, about 20 nM, about 30 nM, about 40 nM, about 50 nM, about 60 nM, about 70 nM, about 80 nM, about 90 nM, or about 100 nM). In some embodiments, the anti-TfR antibody antigen binding domain binds the apical domain of human TfR with an affinity of about 1 nM to about 1000 nM (e.g., about 1 nM, about 2 nM, about 5 nM, about 10 nM, about 20 nM, about 30 nM, about 40 nM, about 50 nM, about 75 nM, about 100 nM, about 150 nM, about 200 nM, about 250 nM, about 300 nM, about 400 nM, about 500 nM, about 750 nM, or about 1000 nM). In some embodiments, the anti-TfR antibody antigen binding domain binds the apical domain of human TfR with an affinity of about 1 nM to about 500 nM. In some embodiments, the anti-TfR antibody antigen binding domain binds the apical domain of human TfR with an affinity of about 1 nM to about 100 nM (e.g., about 1 nM, about 2 nM, about 5 nM, about 10 nM, about 20 nM, about 30 nM, about 40 nM, about 50 nM, about 60 nM, about 70 nM, about 80 nM, about 90 nM, or about 100 nM). In some embodiments, the anti-TfR antibody antigen binding domain binds TfR or the apical domain or TfR with affinity that is less than 1 nM.

### Illustrative Proteins Comprising Non-Targeting Fabs Fragments

In some embodiments, a TfR binder comprise a non-binding Fab or NBVR.

In some embodiments, a non-binding Fab or portion thereof comprises a non-binding variable region (NBVR). A NBVR comprise a light chain variable region and a heavy chain variable region and does not specifically bind to a naturally occurring epitope in a subject. In some embodiments, a NBVR does not specifically bind to an antigen expressed in a given mammal, mammalian tissue, or mammalian cell type. The antigen can be a mammalian antigen or an antigen found in the mammal such as from an infectious organism such as a virus, bacteria, fungus, or parasite. The mammal can be, but is not limited to, a non-human primate, a human, or a rodent (e.g., a mouse). An NBVR can be, but is not limited to a scFv.

Specific binding of an antibody to an antigen means an affinity of at least 10⁶ M⁻¹. Specific binding is detectably higher in magnitude and distinguishable from non-specific binding occurring to at least one unrelated target. Nonspecific binding is often the result of van der Waals forces. Non-binding does not imply the NBVR does not bind any antigen with any affinity. Rather, in some embodiments, a NBVR does not exhibit specific binding to (a) any protein or epitope in mammalian cell, mammalian tissue, or mammal; (b) any surface accessible protein or epitope on a mammalian cell or mammalian tissue; or (c) any serum accessible protein or epitope in a mammalian tissue, or mammal.

A NBVR can be part of an scFv or Fab. A Fab may or may not contain all or part of an antibody hinge region. NBVRs can be produced by recombinant DNA techniques, by enzymatic or chemical separation of intact immunoglobulins, or by chemical peptide synthesis. In some embodiments, an NBVR is part of a non-biding Fab, which comprises a light chain and a heavy chain, wherein the light chain comprises a VL region and a light chain constant region (CL) and the heavy chain comprises a VH region and a heavy chain CH1 constant region.

Exemplary NBVRs include NBVR1 or NBVR2. Unless otherwise apparent from context, reference to NBVR1 or NBVR2 should be understood as referring to any of mouse, chimeric, veneered, humanized, and modified forms of the NBVR1 or NBVR2.

Exemplary NTFs include NBVR1 or NBVR2. Unless otherwise apparent from context, reference to NBVR1 or NBVR2 should be understood as referring to any of mouse, chimeric, veneered, humanized, and modified forms of the NBVR1 or NBVR2.

The sequences of the light and heavy chain variable regions of NBVR1 are designated SEQ ID NOs: 35 and 36, respectively. The sequences of the light and heavy chains of NBVR1 are designated SEQ ID NOs: 37 and 38, respectively.

In some embodiments, a NBVR comprises the CDR sequences of NBVR1. The CDRs (L1, L2, and L3) of the light chain of NBVR1 are designated SEQ ID NOs: 39, 41, and 43, respectively. The CDRs (H1, H2, and H3) of the heavy chain of NBVR1 are designated SEQ ID NOs: 45, 47, and 49, respectively. In some embodiments, a NBVR comprises the CDR-L1, CDR-L2, CDR-L3, CDR-H1, CDR-H2, and CDR-H3 sequences of NBVR1 and a CDR-H2 sequence comprising SEQ ID NO: 50.

In some embodiments, a NBVR comprises a light chain comprising the amino acid sequence of SEQ ID NO: 37 or 52, and a heavy chain comprising the amino acid sequence of SEQ ID NOs: 38, 53, 54, 55, 56, 57, 58, or 59.

In some embodiments, a NBVR comprises a light chain containing an amino acid sequence that is at least 70%, at least 80%, at least 90%, at least 95%, at least 96%, at least 97%, at least 98%, or at least 99% identical to the amino acid sequence of SEQ ID NOs: 35, 37, or 51, and a heavy chain containing an amino acid sequence that is at least 70%, at least 80%, at least 90%, at least 95%, at least 96%, at least 97%, at least 98%, or at least 99% identical to the amino acid sequence of SEQ ID NOs: 36, 38, 53, 54, 55, 56, 57, 58, or 59, and contains the CDR sequences of the NBVR1 and maintains the non-binding properties of NBVR1.

In some embodiments, a NBVR comprises light chain and heavy chain variably regions that differ from NBVR1 light chain and heavy chain variably regions by a small number of functionally inconsequential amino acid substitutions (e.g., conservative substitutions), deletions, or insertions. NBVRs having at 1, 2, 3, 4, 5, or 6 CDR(s) as defined by any conventional definition, but preferably Kabat, that are 90%, 95%, 99% or 100% identical to corresponding CDRs of NBVR1 or NBVR2 are also included.

The sequences of the light and heavy chain variable regions of NBVR2 are designated SEQ ID NOs: 53 and 60, respectively. The sequences of the light and heavy chains of NBVR2 are designated SEQ ID NOs: 53 and 61, respectively.

In some embodiments, a NBVR comprises the CDR sequences of NBVR2. The CDRs (L1, L2, and L3) of the light chain of NBVR2 are designated SEQ ID NOs: 40, 42, and 44, respectively. The CDRs (H1, H2, and H3) of the heavy chain of NBVR2 are designated SEQ ID NOs: 46, 48, and 49, respectively. In some embodiments, a NBVR comprises the CDR-L1, CDR-L2, CDR-L3, CDR-H1, CDR-H2, and CDR-H3 sequences of NBVR2 and a CDR-H2 sequence comprising SEQ ID NO: 50.

In some embodiments, a NBVR comprises a light chain containing an amino acid sequence that is at least 70%, at least 80%, at least 90%, at least 95%, at least 96%, at least 97%, at least 98%, or at least 99% identical to the amino acid sequence of SEQ ID NOs: 62, 63, or 64, and a heavy chain containing an amino acid sequence that is at least 70%, at least 80%, at least 90%, at least 95%, at least 96%, at least 97%, at least 98%, or at least 99% identical to the amino acid sequence of SEQ ID NOs: 60, 61, 65, 66, 67, 68, 69, 70, or 71, and contains the CDR sequences of the NBVR2 and maintains the non-binding properties of NBVR2.

In some embodiments, a NBVR comprises light chain and heavy chain variably regions that differ from NBVR2 light chain and heavy chain variably regions by a small number of functionally inconsequential amino acid substitutions (e.g., conservative substitutions), deletions, or insertions. NBVRs having at 1, 2, 3, 4, 5, or 6 CDR(s) as defined by any conventional definition, but preferably Kabat, that are 90%, 95%, 99% or 100% identical to corresponding CDRs of NBVR1 or NBVR2 are also included.

In some embodiments, a NBVR comprises light and heavy chain variable regions having some or all (e.g., 3, 4, 5, and 6) CDRs entirely or substantially from NBVR1 or NBVR2. Such NBVRs can include a heavy chain variable region that has at least two, and usually all three, CDRs entirely or substantially from the heavy chain variable region of NBVR1 or NBVR2 and/or a light chain variable region having at least two, and usually all three, CDRs entirely or substantially from the light chain variable region of NBVR1 or NBVR2. A CDR is substantially from a corresponding NBVR1 or NBVR2 CDR when it contains no more than 4, 3, 2, or 1 substitutions, insertions, or deletions, except that CDR-H2 (when defined by Kabat) can have no more than 6, 5, 4, 3, 2, or 1 substitutions, insertions, or deletions. Such antibodies can have at least 70%, at least 80%, at least 90%, at least 95%, at least 96%, at least 97%, at least 98%, or at least 99% identity to any of the described NBVR1 or NBVR2 light chain and heavy chain amino acid sequences and maintain their functional properties, and/or differ from NBVR1 or NBVR2. In some embodiments, a NBVR does not exhibit specific binding to (a) any protein or epitope in naturally occurring in mammalian cell, mammalian tissue, or mammal; (b) any surface accessible protein or epitope on a naturally occurring mammalian cell or mammalian tissue; or (c) any serum accessible protein or epitope in a naturally occurring mammalian tissue, or mammal.

In some embodiments, a nucleic acid encoding a NBVR light chain comprises a nucleotide sequence encoding the amino acid sequence of SEQ ID NO: 35, 37, 52, 62, 63, or 64. In some embodiments, a nucleic acid encoding a NBVR heavy chain comprises a nucleotide sequence encoding the amino acid sequence of SEQ ID NO: 36, 38, 53, 54, 55, 56, 57, 58, 59, 60, 61, 65, 66, 67, 68, 69, 70, or 71.

In some embodiments, a nucleic acid encoding a NBVR light chain comprises a nucleotide sequence having at least 70%, at least 75%, at least 80%, at least 85%, at least 90%, at least 95%, at least 96%, at least 97%, at least 98%, or at least 99%, or 100% identity to the nucleotide sequence of SEQ ID NOs: 72 or 73. In some embodiments, a nucleic acid encoding a NBVR heavy chain comprises a nucleotide sequence having at least 70%, at least 75%, at least 80%, at least 85%, at least 90%, at least 95%, at least 96%, at least 97%, at least 98%, or at least 99%, or 100% identity to the nucleotide sequence of SEQ ID NO: 74 or 75.

Described are cells containing nucleic acids encoding the heavy and light chains of any of the described NBVRs. In some embodiments, the cell contains a nucleic acid encoding a NBVR light chain comprising the amino acid sequence of SEQ ID NO: 35, 37, or 52, and a nucleic acid encoding a NBVR heavy chain comprising the amino acid sequence of SEQ ID NO: 36, 38, 53, 54, 55, 56, 57, 58, or 59. In some embodiments, the cell contains a nucleic acid encoding a NBVR light chain comprising the amino acid sequence of SEQ ID NO: 62, 63, or 64, and a nucleic acid encoding a NBVR heavy chain comprising the amino acid sequence of SEQ ID NO: 60, 61, 65, 66, 67, 68, 69, 70, or 71. The cell can be a bacterial cell, a yeast cell, an insect cell or a mammalian cell.

In some embodiments, the non-binding Fab is RSV (palivizumab) Fab fragments (light chain comprises SEQ ID NO: 101), which are non-targeting in mice and non-human primates.

Anti-TfR antibody in which one Fab arm is removed or replaced with a non-binding Fab or NBVR.

### Humanized Antibody Antigen Binding Domains

Any of the described anti-TfR antibody antigen binding domains, non-binding Fabs, NBVRs, or antibodies described herein may be humanized. Humanized antibody antigen binding domains can be humanized in one or more of: a light chain variable domain, a heavy chain variable domain, a light chain constant domain, and a heavy chain constant (CH1) domain. A humanized antibody antigen binding domain is a genetically engineered antibody antigen binding domain in which CDRs from a non-human “donor” antibody are grafted into human “acceptor” antibody heavy and/or light chain variable region, light chain constant region and/or heavy chain CH1 region sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557). The acceptor antibody sequences can be, for example, a mature human antibody sequences (e.g., sequences from one or more of: CH1 region, CH2 region, CH3 region, heavy chain variable region, light chain constant region, or light chain variable region), a composite of such sequences, a consensus sequence of human antibody sequences, or a germline region sequence. Thus, a humanized antibody antigen binding domain is an antibody antigen binding domain having at least three, four, five or all CDRs entirely or substantially from a donor antibody and entirely or substantially human antibody variable region framework sequences and/or constant region sequences. Similarly, a humanized heavy chain has at least one, two and usually all three CDRs entirely or substantially from a donor antibody heavy chain, and a heavy chain variable region framework sequence and heavy chain constant region sequences, if present, substantially from human heavy chain variable region framework and constant region sequences. Similarly, a humanized light chain has at least one, two and usually all three CDRs entirely or substantially from a donor antibody light chain, and a light chain variable region framework sequence and light chain constant region sequences, if present, substantially from human light chain variable region framework and constant region. A CDR in a humanized antibody is substantially from a corresponding CDR in a non-human antibody when at least 85%, 90%, 95% or 100% of corresponding residues (as defined by any conventional definition but preferably defined by Kabat) are identical between the respective CDRs. The variable region framework sequences of an antibody chain or the constant region of an antibody chain are substantially from a human variable region framework sequence or human constant region respectively when at least 85%, 90%, 95% or 100% of corresponding residues defined by Kabat are identical.

In some embodiments, the Fab is a chimeric Fab. A chimeric Fab comprises a non-human light and/or heavy chain variable region and a human heavy chain (CH1) and/or light chain constant region.

In some embodiments, the Fab is a veneered Fab. A veneered Fab comprises a partially humanized light and/or heavy chain variable region and a human heavy chain (CH1) and/or light chain constant region.

### VII. Fc Polypeptide OR Fc Dimer

In some embodiments, a TfR binder comprises a Fc polypeptide or a Fc dimer. A Fc polypeptide or a Fc dimer may comprise one or more mutations or substitutions to increase serum stability, modulate effector function, influence glycosylation, reduce immunogenicity in humans, facilitate heterodimerization (e.g., knob and hole mutations), and/or facilitate conjugation of the oligonucleotide.

In some embodiments, a Fc polypeptide as described herein has an amino acid sequence identity of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% to a corresponding wild-type Fc polypeptide (e.g., a human IgG1, IgG2, IgG3, or IgG4 Fc polypeptide).

One or both of the Fc polypeptides may each comprise independently selected modifications (e.g., mutations) or one or both of the Fc polypeptides may be a wild-type Fc polypeptide, e.g., a human IgG1 Fc polypeptide. In some embodiments, a Fc polypeptide as described herein has an amino acid sequence identity of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% to a corresponding wild-type Fc polypeptide (e.g., a human IgG1, IgG2, IgG3, or IgG4 Fc polypeptide). Non-limiting examples of mutations that can be introduced into one or both Fc polypeptides include certain mutations, e.g., to provide for knob and hole heterodimerization of the polypeptide, to modulate effector function, to extend serum half-life, to influence glycosylation, and/or to reduce immunogenicity in humans.

### Fc Polypeptide Modifications for Heterodimerization

In some embodiments, the Fc polypeptides of a Fc dimer include mutations to promote heterodimer formation and hinder homodimer formation. These modifications are useful, for example, where it is desired to have only one of the Fc polypeptides of a dimer having a TfR binding site (i.e., a monovalent TfR binder).

In some embodiments, polypeptides present in an Fc dimer may include knob and hole mutations to promote heterodimer formation. Generally, the method involves introducing a protuberance (“knob”) at the interface of one polypeptide and a corresponding cavity (“hole”) in the interface of the other polypeptide. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

The knobs-into-holes approach generally involves introducing a protuberance (“knob”) at the interface of one Fc polypeptide and a corresponding cavity (“hole”) in the interface of the other Fc polypeptide, such that the protuberance can be positioned in the cavity so as to promote heterodimer formation and thus hinder homodimer formation. Protuberances are constructed by replacing small amino acid side chains from the interface of the one Fc polypeptide with larger side chains (e.g., Tyr or Trp). Compensatory cavities of identical or similar size to the protuberances are created in the interface of the other Fc polypeptide by replacing large amino acid side chains with smaller ones (e.g., Ala or Thr). In some embodiments, such additional mutations are at a position in the Fc polypeptide that does not have a negative effect on binding of the polypeptide to TfR.

In one illustrative embodiment of a knob and hole approach for dimerization, position 366 of one of the Fc polypeptides comprises a Trp in place of a native Thr. The other Fc polypeptide in the dimer has a Val at position 407 in place of the native Tyr. The other Fc polypeptide may further comprise a substitution in which the native Thr at position 366 is substituted with a Ser and a native Leu at position 368 is substituted with an Ala. Thus, one of the Fc polypeptides has the T366W knob mutation and the other Fc polypeptide has the Y407V hole mutation, which is typically accompanied by the T366S and L368A hole mutations. As indicated above, all positions are numbered per EU numbering. In certain embodiments, first Fc polypeptide comprises T366S, L368A, and Y407V substitutions, according to EU numbering, and the second Fc polypeptide further comprises a T366W substitution, according to EU numbering.

In some embodiments, one or both Fc polypeptides present in an Fc polypeptide dimer can also be engineered to contain other modifications for heterodimerization, e.g., electrostatic engineering of contact residues within a CH3-CH3 interface that are naturally charged or hydrophobic patch modifications.

### Fc Polypeptide Modifications for Modulating Effector Function

In some embodiments, one or both Fc polypeptides in an Fc polypeptide dimer can comprise modifications that reduce effector function, i.e., having a reduced ability to induce certain biological functions upon binding to an Fc receptor expressed on an effector cell that mediates the effector function. Effector cells include, but are not limited to, monocytes, macrophages, neutrophils, dendritic cells, eosinophils, mast cells, platelets, B cells, large granular lymphocytes, Langerhans' cells, natural killer (NK) cells, and cytotoxic T cells. Examples of antibody effector functions include, but are not limited to, C1q binding and complement dependent cytotoxicity (CDC), Fc receptor binding, antibody-dependent cell-mediated cytotoxicity (ADCC), antibody-dependent cell-mediated phagocytosis (ADCP), down-regulation of cell surface receptors (e.g., B cell receptor), and B-cell activation.

In some embodiments, one or both Fc polypeptides in an Fc polypeptide dimer can comprise modifications that reduce or eliminate effector function. Illustrative Fc polypeptide mutations that reduce effector function include, but are not limited to, substitutions in a CH2 domain, e.g., at positions 234 and 235 and/or at position 329, according to the EU numbering scheme. For example, in some embodiments, both Fc polypeptides comprise Ala residues at positions 234 and 235 (also referred to as “LALA” herein). In some embodiments, both Fc polypeptides comprise Gly residue at position 329 (also referred to as “P329G” or “PG” herein) or Ser residue at position 329 (also referred to as “P329S” or “PS” herein). In some embodiments, both Fc polypeptides comprise Ala residues at positions 234 and 235, and Gly residue at position 329 (also referred to as “LALA PG” herein). In some embodiments, both Fc polypeptides comprise Ala residues at positions 234 and 235, and Ser residue at position 329 (also referred to as “LALA PS” herein).

Additional Fc polypeptide mutations that modulate an effector function include, but are not limited to, the following: position 329 may have a mutation in which Pro is substituted with a Gly, Ala, Ser, or Arg or an amino acid residue large enough to destroy the Fc/Fcγ receptor interface that is formed between proline 329 of the Fc and Trp residues Trp87 and Trp110 of FcγRIII. Additional illustrative substitutions include S228P, E233P, L235E, N297A, N297D, and P331S, according to the EU numbering scheme. Multiple substitutions may also be present, e.g., L234A, L235A, and P329G of human IgG1; S228P and L235E of human IgG4; L234A and G237A of human IgG1; L234A, L235A, and G237A of human IgG1; V234A and G237A of human IgG2; L235A, G237A, and E318A of human IgG4; and S228P and L236E of human IgG4, according to the EU numbering scheme.

### Fc Polypeptide Modifications for Extending Serum Half-Life

In some embodiments, modifications to enhance serum half-life can be introduced into any Fc polypeptides described herein. For example, in some embodiments, both Fc polypeptides in an Fc polypeptide dimer can comprise M428L and N434S substitutions (also referred to as LS substitutions), as numbered according to the EU numbering scheme. Alternatively, both Fc polypeptides in an Fc polypeptide dimer can have an N434S or N434A substitution. Alternatively, both Fc polypeptides in an Fc polypeptide dimer can have an M428L substitution. In other embodiments, both Fc polypeptides in an Fc polypeptide dimer can comprise M252Y, S254T, and T256E substitutions.

Fc Polypeptide with C-Terminal Lysine Residue Removed

In some embodiments, one or both of the Fc polypeptides can have its C-terminal lysine removed (e.g., the Lys residue at position 447 of the Fc polypeptide, according to EU numbering). The C-terminal lysine residue is highly conserved in immunoglobulins across many species and may be fully or partially removed by the cellular machinery during protein production. In some embodiments, removal of the C-terminal lysines in the Fc polypeptides can improve the stability of the proteins.

Exemplary Fc polypeptides are provided in SEQ ID NOs: 76-100.

### Engineered Anti-TfR Antibody Variants for Conjugation Via a Linking Group

As described herein, a TfR binding antibody (or other TfR binder—e.g., a monovalent anti-TfR antibody, an anti-TfR/non-binding Fab bispecific antibody, or an anti-TfR/NBVR bispecific antibody—as described herein) may be linked to an oligonucleotide(s) through a linking group “L”. In one aspect, the antibody comprises one or more amino acid residues (e.g., amino acid residues that are present at accessible sites in the antibody), which may be used to attach the antibody to L. For example, in one aspect, the antibody comprises one or more cysteine residues (e.g., cysteine residues that are present at accessible sites in the antibody). In certain embodiments, the antibody is attached to L through a cysteine residue of the antibody (e.g., through a sulfur atom of a cysteine residue). In some embodiments, the cysteine is a cysteine modification, wherein an amino acid residue other than cysteine that is present at an accessible site in the antibody is modified to cysteine. In other embodiments, the antibody comprises one or more glutamine residues. In certain embodiments, the antibody is attached to L through a glutamine residue (e.g., through an amide bond in the side chain of a glutamine residue).

In other aspects, it may be desirable to create engineered antibodies with one or more modified sites. These modified sites may be used to facilitate the attachment of a TfR binder to each L. For example, a TfR binder may be attached to each L at the modified site. In other embodiments, the modified site may enable the attachment of L to an amino acid residue located near the modified site (e.g., within 1, 2, 3, 4 5, 6, 7, 8, 9 or 10 amino acids of the modified site, such as within 2 or 3 amino acids of the modified site). In particular embodiments, such modified sites are substituted residues that occur at accessible sites of the antibody. In certain embodiments, an anti-TfR antibody (e.g., a monovalent anti-TfR antibody, an anti-TfR/non-binding Fab bispecific antibody, or an anti-TfR/NBVR bispecific antibody) described herein comprises one or more modified sites (e.g., one or more amino acid substitutions, such as a cysteine, alanine or glycine substitution). In certain embodiments, the antibody comprises at least or exactly 1, 2, 3, 4, 5, 6, 7, or 8 modified sites. In certain embodiments, the antibody comprises 1 to 8, 1 to 7, 1 to 6, 1 to 5, 1 to 4, 1 to 3, or 1 to 2 modified sites. In certain embodiments, the antibody comprises 2 to 4 modified sites.

In certain embodiments, a modified site within the antibody is an amino acid substitution or insertion. In certain embodiments, the antibody comprises (or is a protein that is) an Fc dimer In certain embodiments, the Fc polypeptide(s) may be part of a Fab-Fc fusion or Fab-Fc dimer fusion and the modified site may be in a Fab-Fc polypeptide that binds TfR and/or in a Fab-Fc polypeptide that has formed a dimer with a Fab-Fc polypeptide that binds TfR.

In certain embodiments, a modified site is present in a CL domain. In certain embodiments, a modified site is present in a CH1 domain. In certain embodiments, a modified site is present in a CH2 domain. In certain embodiments, a modified site is present in a CH3 domain.

In certain embodiments, the modified site is an amino acid substitution. In certain embodiments, the modified site is a cysteine, glycine or alanine substitution.

In certain embodiments, the modified site is a cysteine substitution. By substituting those residues with cysteine, reactive thiol groups are thereby positioned at accessible sites of the antibody and may be used to conjugate the antibody to an oligonucleotide via a linking group (L), to create a conjugate as described herein. In certain embodiments, the antibody contains an Fc polypeptide or Fc polypeptide dimer and includes a cysteine substitution selected from the group consisting of S239C, S442C, A330C, and T289C, wherein the positions and substitutions are according to EU numbering. In other embodiments, the Fc polypeptide is joined to CH1 domain and includes an A114C substitution. In other embodiments, the comprises a Fab-Fc fusion, and the light chain includes a K149C substitution.

In other aspects, the modified site is an alanine or glycine substitution. Such modified amino acids may facilitate enzymatic conjugation of L to the antibody at a nearby amino acid, such as a glutamine residue (e.g., using bacterial transglutaminase (BTG). For example, in certain embodiments, the alanine/glycine substitution is N297A or N297G, wherein the positions and substitutions are according to EU numbering. These substitutions eliminate glycosylation at position 297, which would hinder enzymatic conjugation of the linker to the antibody at position Q295 (i.e., the linker is attached to the antibody through an amide bond in the side chain of the glutamine). Thus, in certain embodiments, the modified site is N297A or N297G and the antibody is attached to L at Q295 (e.g., by enzymatic conjugation).

In certain embodiments, the N-terminus of the Fc polypeptide includes a portion of the hinge region (e.g., DKTHTCP (SEQ ID NO: 4 or DKTHTCPPCP (SEQ ID NO: 5)).

In some embodiments the TfR-binder oligonucleotide conjugate comprises a Fc polypeptide or a Fc dimer. An Fc dimer comprises a first Fc polypeptide and a second Fc polypeptide. In certain embodiments, the Fc polypeptide or first Fc polypeptide comprises one or more amino acid substitutions (e.g., 1 or more cysteine substitutions). In certain embodiments, the Fc polypeptide or first Fc polypeptide comprises one or more substitutions selected from the group consisting of S239C, S442C, A330C, T289C, N297A and N297G in the heavy chain (according the EU numbering), K149C in the light chain (according to EU numbering), and A114C in the heavy chain (according to Kabat numbering). In certain embodiments, the Fc polypeptide or first Fc polypeptide comprises S239C. In certain embodiments, the Fc polypeptide or first Fc polypeptide comprises S442C. In certain embodiments, the Fc polypeptide or first Fc polypeptide comprises A330C. In certain embodiments, the Fc polypeptide or first Fc polypeptide comprises T289C. In certain embodiments, the Fc polypeptide or first Fc polypeptide comprises N297A. In certain embodiments, the Fc polypeptide or first Fc polypeptide comprises N297G. In certain embodiments, the Fc polypeptide or first Fc polypeptide comprises S239C and A330C.

In certain embodiments, the second Fc polypeptide (of the Fc dimer) comprises one or more amino substitutions (e.g., 1 or more cysteine substitutions). In certain embodiments, the second Fc polypeptide comprises one or more substitutions selected from the group consisting of S239C, S442C, A330C, T289C, N297A and N297G in the heavy chain (according to EU numbering), K149C in the light chain (according to EU numbering), and A114C in the heavy chain (according to Kabat numbering). In certain embodiments, the second Fc polypeptide comprises S239C. In certain embodiments, the second Fc polypeptide comprises S442C. In certain embodiments, the second Fc polypeptide comprises A330C. In certain embodiments, the second Fc polypeptide comprises T289C. In certain embodiments, the second Fc polypeptide comprises N297A. In certain embodiments, the second Fc polypeptide polypeptide comprises N297G. In certain embodiments, the second Fc polypeptide comprises S239C and A330C. In certain embodiments, the second Fc polypeptide comprises A114C.

In certain embodiments, a Fc polypeptide comprises a sequence having at least 85% identity, at least 90% identity, at least 95% identity, at least 96% identity, at least 97% identity, at least 98% identity, or at least 99% to SEQ ID NO: 1.

In certain embodiments, the first Fc polypeptide and the second Fc polypeptide of a Fc dimer each comprise one or more amino acid substitutions (e.g., 1 or more cysteine substitutions). In certain embodiments, the one or more substitutions are: S239C, S442C, A330C, T289C, N297A and/or N297G, according to EU numbering, and/or A114C according to Kabat numbering. In certain embodiments, the one or more substitutions are: S239C, S442C, A330C, A114C, and/or T289C. In certain embodiments, the one or more substitutions are: S239C, S442C, A114C, and/or T289C. In certain embodiments, the one or more substitutions are: N297A and/or N297G. In certain embodiments, the first Fc polypeptide and the second Fc polypeptide each comprise one amino acid substitution to facilitate conjugation of the oligonucleotide (e.g., 1 cysteine substitution). In certain embodiments, the first and second Fc polypeptides each comprise a cysteine substitution at S239C In certain embodiments, the first and second Fc polypeptides each comprise two amino acid substitutions (e.g., 2 cysteine substitutions). In certain embodiments, the first and second Fc polypeptides each comprise a cysteine substitution at S239C and A330C.

Fc polypeptides or dimers thereof, that comprise one or more modified sites (e.g., cysteine substitutions) may be used in a conjugate as described herein.

In some embodiments, the anti-TfR antibody antigen binding domain comprises a Fab or scFab, wherein the Fab or scFab comprises a K149C substitution on the light chain (according to EU numbering) or a A114C substitution on the heavy chain (according to Kabat numbering).

In certain embodiments, 1 or more oligonucleotides are attached to the linking group (L). In certain embodiments, 2 or more oligonucleotides are attached to the linking group (L). In certain embodiments, 1 oligonucleotide is attached to the linking group (L). In certain embodiments, 2 oligonucleotides are attached to the linking group (L).

### VIII. Albumin

For TfR binder-oligonucleotide conjugates comprising albumin, the albumin can be human albumin or albumin from another mammalian species, such as, but not limited to, a mouse albumin or a non-human primate albumin. In some embodiments, the albumin is a human albumin (SEQ ID NO: 167; UNIPROT accession P0276, GenBank: AAA98797.1, NCBA NP_000468.1, Gene ID: 213, mRNA NM_000477.7). The oligonucleotide can be linked to the albumin, optionally via a linking group, to a surface accessible free cysteine in the albumin (e.g., C58 of mouse preproalbumin (position 34 (boxed) of SEQ ID NOs: 167 and 168). The albumin may be modified to contain one or more amino acid substitutions, such as a cysteine substitution, to facilitate conjugation to the oligonucleotide.

In some embodiments, a TfR binder-oligonucleotide conjugate comprises an anti-TfR scFv fused to an albumin protein. The anti-TfR scFv fused to the albumin protein can be provided as a single polypeptide chain fusion protein. The anti-TfR scFv can be fused to the amino or carboxy terminal end of the albumin. In some embodiments, a anti-TfR scFv is fused to the amino terminal end of an albumin. The fusion portion can contain a linking peptide between the scFc and the albumin. The linking peptide can be, but is not limited to, a GGGS (Glycine) 3-Serine) peptide. Exemplary anti-TfR scFv-albumin fusion proteins are provided in SEQ ID NOs: 169 and 170, which contain a 17H10 anti-TfR scFv fused to a mouse and human albumin, respectively. The anti-TfR scFv-albumin fusion protein can further contain a peptide that facilitates purification, such as an epitope tag or a polyhistidine tag (e.g., His₆). The epitope tag or polyhistidine can be located at the amino terminal or carboxy terminal end of the fusion protein.

TABLE 2
Human albumin amino acid sequence (SEQ ID NO: 167)

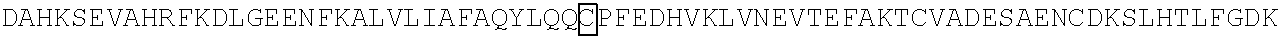

LCTVATLRETYGEMADCCAKQEPERNECFLOHXDDNPNLPRLVRPEVDVMCTAFHDNEETFLKKYLYEIARRH
PYFYAPELLFFAKRYKAAFTECCOAADKAACLLPKLDELRDEGKASSAKORLKCASLOKFGERAFKAWAVARL
SQRFPKAEFAEVSKLVTDLTKVHTECCHGDLLECADDRADLAKYICENQDSI SSKLKECCEKPLLEKSHCIAE
VENDEMPADLPSLAADFVESKDVCKNYAEAKDVFLGMFLYEYARRHPDYSVVLLLRLAKTYETTLEKCCAAAD
PHECYAKVEDEFKPLVEEPONLIKONCELFEQLGEYKFQNALLVRYTKKVPQVSTPTLVEVSRNLGKVGSKCC
KHPEAKRMPCAEDYLSVVLNQLCVLHEKTPVSDRVTKCCTESLVNRRPCESALEVDETYVPKEFNAETFTFHA
DICTLSEKERQIKKQTALVELVKHKPKATKEOLKAVMDDFAAFVEKCCKADDKETCFAEEGKKLVAASQAALG
L
Mouse albumin amino acid sequence (SEQ ID NO: 168)

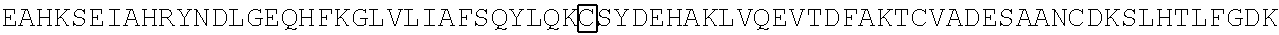

LCAIPNLRENYGELADCCTKQEPERNECFLOHKDDNPSLPPFERPEAEAMCTSFKENPTTEMGHYLHEVARRH
PYFYAPELLYYAEQYNEILTOCCAEADKESCLTPKLDGVKEKALVSSVRORMKCSSMOKEGERAFKAWAVARL
SQTFPNADFAEITKLATDLTKVNKECCHGDLLECADDRAELAKYMCENOATISSKLOTCCDKPLLKKAHCLSE
VEHDTMPADLPAIAADFVEDQEVCKNYAEAKDVELGTFLYEYSRRHPDYSVSLLLRLAKKYEATLEKCCAEAN
PRACYGTVLAEFQPLVEEPKNIVKINCDLYEKLGEYGFQNAILVRYTQKAPQVSTPTIVEAARNLGRVGTKCC
TLPEDQRLPCVEDYLSAILNRVCLLHEKTPVSEHVTKCCSGSLVERRPCFSALTVDETYVPKEFKAETETEHS
DICTLPEKEKQIKKOTALAELVKHKPKATAEQLKTVMDDFAQFLDTCCKAADKDTCFSTEGPNLVTRCKDALA
17h10 anti-TfR scFv-malbumin fusion protein (SEQ ID NO: 169)
EVOLVESGGGLVQPGGSLRLSCAVSGFSLTTYGVDWVRQAPGKGLEWVGIIWGDGTTHYHSALISRFTISKDN
SKNTVYLOMNSLRAEDTAVYYCAKPGGASWGQGTLVTVSSGGGGSGGGGSGGGGSDIQMTOSPSSLSASVGDR
VTITCKASQNVGTNVAWYQQKPGKSPKALFYSASYRNAGVPSRESGSGSGTDFTLTISSLOPEDFATYFCQHY

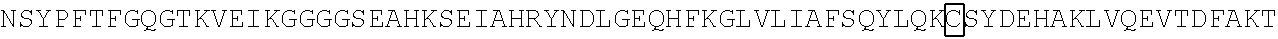

CVADESAANCDKSLHTLFGDKLCAIPNLRENYGELADCCTKQEPERNECFLQHKDDNPSLPPFERPEAEAMCT
SEKENPTTEMGHYLHEVARRHPYFYAPELLYYAEQYNEILTOCCAEADKESCLTPKLDGVKEKALVSSVRORM
KCSSMOKEGERAFKAWAVARLSQTFPNADFAEITKLATDLTKVNKECCHGDLLECADDRAELAKYMCENQATI
SSKLQTCCDKPLLKKAHCLSEVEHDTMPADLPAIAADFVEDQEVCKNYAEAKDVELGTFLYEYSRRHPDYSVS
LLLRLAKKYEATLEKCCAEANPPACYGTVLAEFOPLVEEPKNLVKINCDLYEKLGEYGFQNAILVRYTQKAPQ
VSTPTIVEAARNLGRVGTKCCTLPEDORLPCVEDYLSAILNRVCLLHEKTPVSEHVTKCCSGSLVERRPCFSA
LTVDETYVPKEFKAETFTFHSDICTLPEKEKQIKKOTALAELVKHKPKATAEQLKTVMDDFAQFLDTCCKAAD
KDTCFSTEGPNLVTRCKDALA
17h10 anti-TER scFv-halbumin fusion protein (SEQ ID NO: 170)
EVOLVESGGGLVQPGGSLRLSCAVSGFSLTTYGVDWVRQAPGKGLEWVGIIWGDGTTHYHSALISRFTISKDN
SKNTVYLQMNSLRAEDTAVYYCAKPGGASWGQGTLVTVSSGGGGSGGGGSGGGGSDIQMTQSPSSLSASVGDR
VTITCKASQNVGTNVAWYQQKPGKSPKALFYSASYRNAGVPSRESGSGSGTDFTLTISSLOPEDFATYFCQHY

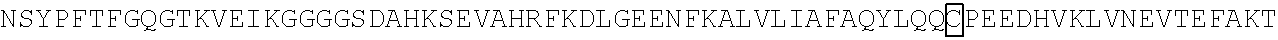

CVADESAENCDKSLHTLFGDKLCTVATLRETYGEMADCCAKQEPERNECFLOHKDDN PNLPRLVRPEVDVMCT
AFHDNEETFLKKYLYEIARRHPYFYAPELLFFAKRYKAAFTECCOAADKAACLLPKLDELRDEGKASSAKORL
KCASLOKFGERAFKAWAVARLSQRFPKAEFAEVSKLVTDLTKVHTECCHGDLLECADDRADLAKYICENQDSI
SSKLKECCEKPLLEKSHCIAEVENDEMPADLPSLAADEVESKDVCKNYAEAKDVELGMELYEYARRHPDYSVV
LLLRLAKTYETTLEKCCAAADPHECYAKVEDEFKPLVEEPONLIKONCELFEOLGEYKFONALLVRYTKKVPQ
VSTPTLVEVSRNLGKVGSKCCKHPEAKRMPCAEDYLSVVLNQLCVLHEKTPVSDRVTKCCTESLVNRRPCFSA
LEVDETYVPKEFNAETFTFHADICTLSEKERQIKKQTALVELVKHKPKATKEQLKAVMDDFAAFVEKCCKADD
KETCFAEEGKKLVAASQAALGL

### IX. NUCLEIC ACIDS, VECTORS, and HOST CELLS

The TfR binders described herein can be prepared using recombinant methods. Accordingly, isolated nucleic acids comprising sequences encoding any of the TfR binders described herein or portions thereof are readily generated using methods available in the art. Host cells into which the nucleic acids are introduced and that can be used to replicate the polypeptide-encoding nucleic acids and/or to express the polypeptides are also available in the art. A host cell can be, but is not limited to, a prokaryotic cell or a eukaryotic, the eukaryotic call be, but is not limited to, a yeast cell, an insect cell, or a mammalian cell (e.g., a human cell).

A nucleic acid encoding a TfR binders or a portion thereof can be DNA, RNA, cDNA, mRNA, single-stranded, double-stranded, linear or circular.

An TfR binders may comprise two or more (e.g., three) polypeptides, each of which may be encoded by a separate nucleic sequence. The separate nucleic acid sequence may be present on the same plasmid or vector or different plasmids or vectors. If present on the same plasmid or vector, the separate nucleic acid sequences may be expressed from a single promoter or from different promoters. Method of expressing nucleic acids encoding separate polypeptides from a single promoter are known in the art and include, but are not limited to, the use of 2A elements and internal ribosome entry sites.

A nucleic acid encoding a TfR binders or a portion thereof can be provided in a plasmid or vector. The plasmid or vector can be used to replicate the nucleic acid or facilitate expression of the nuclei acid. A plasmid or vector can be, but is not limited to, a viral vector, a phagemid, a yeast chromosomal vector, or a non-episomal mammalian vector.

In some embodiments, the nucleic acid encoding a TR binders or a portion thereof operably linked to one or more regulatory sequences in an expression construct. The expression constructs can be adapted for expression of the polypeptide in a system that production of the dual transporter. Such a system can be, but is not limited to, mammalian cell expression system, an insect cell expression system, a yeast cell expression system, or a bacterial cell expression system.

Expression vehicles for production of a recombinant polypeptide include plasmids and other vectors. For instance, suitable vectors include plasmids of the following types: pBR322-derived plasmids, pEMBL-derived plasmids, pEX-derived plasmids, pBTac-derived plasmids, and pETC-derived plasmids for expression in prokaryotic cells, such as E. coli. The pcDNAI/amp, pcDNAEneo, pRc/CMV, pSV2gpt, pSV2neo, pSV2-dhfr, pTk2, pRSVneo, pMSG, pSVT7, pko-neo, and pHyg-derived vectors are examples of mammalian expression vectors suitable for transfection of eukaryotic cells. Alternatively, derivatives of viruses such as the bovine papilloma virus (BPV-1), or Epstein-Barr virus (pHEBo, pREP-derived, and p205) can be used for transient expression of polypeptides in eukaryotic cells. In some embodiments, it may be desirable to express the recombinant polypeptide by the use of a baculovirus expression system. Examples of such baculovirus expression systems include pVL-derived vectors (such as pVL1392, pVL1393, and pVL941), pAcUW-derived vectors (such as pAcUW1), and pBlueBac-derived vectors. Additional expression systems include adenoviral, adeno-associated virus, and other viral expression systems.

An expression vector for expressing a TfR binders or a portion thereof, or a plasmid or vector containing the nucleic acid can be transformed, transfected, or transduced into a host cell. The host cell can be, but is not limited to, a mammalian cell, a yeast cell, an insect cell, prokaryotic cell, Chinese hamster ovary (CHO) cell, a baby hamster kidney (BHK) cell, a NSO cell, a YO cell, a HEK293 cell, a COS cell, a Vero cell, or a HeLa cell. The host cell containing the expression vector can be cultured under appropriate conditions to allow expression of the TfR binders or a portion thereof.

A TfR binders can be manufactured by culturing a host cell comprising one or more nucleic acids encoding the TfR binders, expressing the TfR binders, and isolating the expressed TfR binders from the culture.

### X. Methods of Use

A conjugate as described herein may be used for a variety of purposes, including therapeutic indications.

In some embodiments, the conjugate is used to deliver an oligonucleotide (e.g., an ASO or RNAi agent) to a target cell type that expresses the transferrin receptor. In some embodiments, a conjugate may be used to transport an oligonucleotide (e.g., an ASO or RNAi agent) across an endothelium, e.g., the blood-brain barrier, to be taken up by the brain.

For example, certain embodiments provide a method for transcytosis of an oligonucleotide (e.g., an ASO or an RNAi agent) across an endothelium, the method comprising contacting the endothelium (e.g., blood-brain barrier (BBB)) with a conjugate as described herein. Thus, certain embodiments provide a method of transporting an oligonucleotide across the BBB of a subject in need thereof, comprising administering a conjugate as described herein to the subject. In certain embodiments, a conjugate as described herein for use in transporting an oligonucleotide across the BBB of a subject in need thereof is provided. In certain embodiments, a conjugate as described herein for use in transporting an oligonucleotide to muscle cells of a subject in need thereof is provided.

Certain embodiments also provide a method of modulating the expression of a target gene or sequence in a subject in need thereof, comprising administering an effective amount of a conjugate as described herein to the subject. In some embodiments, a conjugate as described herein in for use modulating the expression of a target gene is provided.

In certain embodiments, the target gene or sequence is expressed in a cell in the brain of a subject. In certain embodiments, the target gene or sequence is expressed in a cell that expresses TfR. In certain embodiments, the target gene or sequence is expressed in a muscle cell, such as a skeletal muscle cell or a cardiac muscle cell.

In certain embodiments, the modulation of the target gene expression is gene knockdown or gene knockout. Thus, in certain embodiments, the expression of the target gene or sequence is inhibited or reduced, e.g., by at least about 10%, 20%, 30%, 40%, 50%, 60%, 70%, 80%, 90%, 95%, 99% or 100%, as compared to the expression in a control (e.g., a subject that was not administered the conjugate).

A conjugate as described herein is administered to a subject at a therapeutically effective amount or dose. The dosages, however, may be varied according to several factors, including the chosen route of administration, the formulation of the composition, patient response, the severity of the condition, the subject's weight, and the judgment of the prescribing physician. The dosage can be increased or decreased over time, as required by an individual patient.

In various embodiments, a conjugate as described herein is administered parenterally. In some embodiments, the conjugate is administered intravenously. Intravenous administration can be by infusion, e.g., over a period of from about 10 to about 30 minutes, or over a period of at least 1 hour, 2 hours, or 3 hours. In some embodiments, the conjugate is administered as an intravenous bolus. Combinations of infusion and bolus administration may also be used.

In some parenteral embodiments, a conjugate is administered intraperitoneally, subcutaneously, intradermally, or intramuscularly. In some embodiments, the conjugate is administered intradermally or intramuscularly. In some embodiments, the conjugate is administered intrathecally, such as by epidural administration, or intracerebroventricularly.

In other embodiments, a conjugate as described herein may be administered orally, by pulmonary administration, intranasal administration, intraocular administration, or by topical administration. Pulmonary administration can also be employed, e.g., by use of an inhaler or nebulizer, and formulation with an aerosolizing agent.

### XI. Pharmaceutical Compositions and Kits

In another aspect, pharmaceutical compositions and kits comprising a conjugate as described herein are provided.

### Pharmaceutical Compositions

Guidance for preparing formulations for use as described herein can be found in any number of handbooks for pharmaceutical preparation and formulation that are known to those of skill in the art.

In some embodiments, a pharmaceutical composition comprises a conjugate as described herein and further comprises one or more pharmaceutically acceptable carriers and/or excipients. In certain embodiments, the composition comprises a plurality of conjugates as described herein, which can be the same or different (e.g., a mixture of different conjugates). In certain embodiments, the ratio of oligonucleotide to protein in the composition is about 1:1 to about 4:1. In certain embodiments, the ratio of oligonucleotide to protein in the composition is about 1:1 to about 2:1. In certain embodiments, the ratio of oligonucleotide to protein in the composition is about 1.23. In certain embodiments, the ratio of oligonucleotide to protein in the composition is about 2:1 to about 3:1. In certain embodiments, the ratio of oligonucleotide to protein in the composition is about 2.5.

As used herein, the term pharmaceutically acceptable carrier includes any solvents, dispersion media, or coatings that are physiologically compatible and that preferably does not interfere with or otherwise inhibit the activity of the active agent. Various pharmaceutically acceptable excipients are well-known. In some embodiments, the carrier is suitable for intravenous, intrathecal, intracerebroventricular, intramuscular, oral, intraperitoneal, transdermal, topical, or subcutaneous administration. Pharmaceutically acceptable carriers can contain one or more physiologically acceptable compounds that act, for example, to stabilize the composition or to increase or decrease the absorption of the conjugate. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are also available in the art.

The pharmaceutical compositions described herein can be manufactured in a manner that is known to those of skill in the art, e.g., by means of conventional mixing, dissolving, granulating, dragee-making, emulsifying, encapsulating, entrapping, or lyophilizing processes. The following methods and excipients are merely exemplary and are in no way limiting.

For oral administration, a conjugate as described herein can be formulated by combining it with pharmaceutically acceptable carriers that are well-known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the conjugates with a solid excipient, optionally grinding a resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone. If desired, disintegrating agents can be added, such as a cross-linked polyvinyl pyrrolidone, agar, or alginic acid or a salt thereof such as sodium alginate.

As disclosed above, a conjugate as described herein can be formulated for parenteral administration by injection, e.g., by bolus injection or continuous infusion. For injection, the conjugates can be formulated into preparations by dissolving, suspending, or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers, and preservatives. In some embodiments, conjugates can be formulated in aqueous solutions, preferably in physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions, or emulsions in oily or aqueous vehicles, and can contain formulatory agents such as suspending, stabilizing, and/or dispersing agents.

Typically, a pharmaceutical composition for use in in vivo administration is sterile. Sterilization can be accomplished according to methods known in the art, e.g., heat sterilization, steam sterilization, sterile filtration, or irradiation.

Dosages and desired drug concentration of pharmaceutical compositions as described herein may vary depending on the particular use envisioned. The determination of the appropriate dosage or route of administration is well within the skill of one in the art. Suitable dosages are also described above.

### Kits

In some embodiments, kits comprising a conjugate as described herein are provided. In some embodiments, the kits are for use in modulating the expression of a target gene or sequence (e.g., a target gene expressed in the brain or central nervous system (CNS)). In some embodiments, the kits are for use in in modulating the expression of a target gene.

In some embodiments, the kit further comprises one or more additional therapeutic agents. For example, in some embodiments, the kit comprises a conjugate as described herein and further comprises one or more additional therapeutic agents. In some embodiments, the kit further comprises instructional materials containing directions (i.e., protocols) for the practice of the methods described herein (e.g., instructions for using the kit for administering a composition across the blood-brain barrier). While the instructional materials typically comprise written or printed materials, they are not limited to such. Any medium capable of storing such instructions and communicating them to an end user is contemplated herein. Such media include, but are not limited to, electronic storage media (e.g., magnetic discs, tapes, cartridges, chips), optical media (e.g., CD-ROM), and the like. Such media may include addresses to internet sites that provide such instructional materials.

TABLE 3
Informal Sequence Listing

| SEQ | | |
|---|---|---|
| ID | | |
| NO. | Sequence | Description |
| 1 | PCPAPELLGGPSVFLFPPKPKDTLMISRTPEVTCVVVDVSHEDPEVKENWYV | Wild-type human Fc |
| | DGVEVHNAKTKPREEQYNSTYRVVSVLTVLHQDWLNGKEYKCKVSNKALPAP | sequence; |
| | IEKTISKAKGQPREPQVYTLPPSRDELTKNQVSLTCLVKGFYPSDIAVEWES | amino acids 1-3 |
| | NGQPENNYKTTPPVLDSDGSFFLYSKLTVDKSRWQQGNVFSCSVMHEALHNH | (PCP) are from a |
| | YTQKSLSLSPGK | hinge region |
| 2 | MMDQARSAFSNLFGGEPLSYTRESLARQVDGDNSHVEMKLAVJEEENADNNT | Human transferrin |
| | KANVTKPKRCSGSICYGTIAVIVEFLIGEMIGYLGYCKGVEPKTECERLAGT | receptor protein 1 |
| | ESPVREEPGEDFPAARRLYWDDLKRKLSEKLDSTDETGTIKLLNENSYVPRE | (TFR1) |
| | AGSQKDENLALYVENQFREFKLSKVWRDQHFVKIQVKDSAQNSVIIVDKNGR | |
| | LVYLVENPGGYVAYSKAATVTGKLVHANFGTKKDFEDLYTPVNGSIVIVRAG | |
| | KITFAEKVANAESLNAIGVLIYMDQTKFPIVNAELSFFGHAHLGTGDPYTPG | |
| | FPSFNHTQFPPSRSSGLPNIPVQTISRAAAEKLEGNMEGDCPSDWKTDSTCR | |
| | MVTSESKNVKLTVSNVLKEIKILNIFGVIKGFVEPDHYVVVGAQRDAWGPGA | |
| | AKSGVGTALLLZLAQMFSDMVLKDGFQPSRSIIFASWSAGDEGSVGATEWLE | |
| | GYLSSLHLKAFTYINLDKAVLGTSNEKVSASPLLYTLIEKTMQNVKHPVTGQ | |
| | FLYQDSNWASKVEKLTLDNAAFPFLAYSGIPAVSFCFCEDTDYPYLGTTMDT | |
| | YKELIERIPELNKVARAAAEVAGQFVIKLTHDVELNLDYERYNSQLLSFVRD | |
| | LNQYRADIKEMGLSLQWLYSARGDFFRATSRLTTDEGNAEKTQRFVMKKLND | |
| | RVMRVEYHFLSPYVSPKESPFRHVFWGSGSHTLPALLENLKLRKQNNGAFNE | |
| | TLFRNQLALATWTIQGAANALSGDVWDIDNEF | |
| 3 | NSVIIVDKNGRLVYLVENPGGYVAYSKAATVTGKLVHANFGTKKDFEDLYTP | Human TfR apical |
| | VNGSIVIVRAGKITFAEKVANAESLNAIGVLIYMDQTKFPIVNAELSFFGHA | domain |
| | HLGTGDPYTPGEPSENHTQFPPSRSSGLPNIPVQTISRAAAEKLEGNMEGDC | |
| | PSDWKTDSTCRMVTSESKNVKLTVS | |
| 4 | DKTHTCP | Portion of human |
| | | IgG1 hinge sequence |
| 5 | DKTHTCPPCP | Portion of human |
| | | IgG1 hinge sequence |
| 6 | EPKSCDKTHTCPPCP | Human IgGI hinge |
| | | amino acid sequence |
| 7 | MMDQARSAFSNLEGGEPLSYTRFSLARQVDGDNSHVEMKLGVDEEENTDNNT | Cyno TfR |
| | KANGTKPKRCGGNICYGTIAVIIFFLIGEMIGYLGYCKGVEPKTECERLAGT | |
| | ESPAREEPEEDEPAAPRLYWDDLKRKLSEKLDTTDFTSTIKLLNENLYVPRE | |
| | AGSQKDENLALYIENQFREFKLSKVWRDQHFVKIQVKDSAQNSVIIVDKNGG | |
| | LVYLVENPGGYVAYSKAATVTGKLVHANEGTKKDFEDLDSPVNGSIVIVRAG | |
| | KITFAEKVANAESLNAIGVLIYMDQTKFPIVKADLSFFGHAHLGTGDPYTPG | |
| | FPSFNHTQFPPSQSSGLPNIPVQTISRAAAEKLFGNMEGDCPSDWKTDSTCK | |
| | MVTSENKSVKLTVSNVLKETKILNIFGVIKGFVEPDHYVVVGAQRDAWGPGA | |
| | AKSSVGTALLLKLAQMFSDMVLKDGFQPSRSIIFASWSAGDEGSVGATEWLE | |
| | GYLSSLHLKAFTYINLDKAVLGTSNFKVSASPLLYTLIEKTMQDVKHPVTGR | |
| | SLYQDSNWASKVEKLTLDNAAFPFLAYSGIPAVSFCFCEDTDYPYLGTTMDT | |
| | YKELVERIPELNKVARAAAEVAGQFVIKLTHDTELNLDYERYNSQLLLFLRD | |
| | LNQYRADVKEMGLSLQWLYSARGDFFRATSRLTTDERNAEKRQKFVMKKLND | |
| | RVMRVEYYFLSPYVSPKESPFRHVFWGSGSHTLSALLESLKLRRQNNSAFNE | |
| | TLFRNQLALATWTIQGAANALSGDVWDIDNEF | |
| 8 | GₖₛₘCₖₛAₖₛT_{ds}T_{dsm}C_{ds}A_{ds}A_{ds}T_{ds}A_{ds}G_{dsm}C_{ds}AₖₛGₖₛₘCₖ | Malat1 ASO |
| | | (mouse) |
| | Bivalent anti-TER antibody | |
| 9 | DIQMTQSPSSLSASVGDRVTITCRTSENIYNNLAWYQQKPGKSPKLLIYAAT | DS05288 |
| | NLADGVPSRESGSGSGTDYTLTISSLQPEDFATYYCQHFWGTPLTFGGGTKV | LightChain 13E4v2i |
| | EIKRTVAAPSVFIFPPSDEQLKSGTASVVCLLNNFYPREAKVQWKVDNALQS | i VL1 |
| | GNSQESVTEQDSKDSTYSLSSTLTLSKADYEKHKVYACEVTHQGLSSPVTKS | |
| | FNRGEC | |
| 10 | QVQLVQSGAEVKKPGASVKVSCKASGYTETNYWMHWVRQAPGQGLEWIGEIN | DS05289 |
| | PINGRSNYAEKFQGRVTLTVDTSSSTAYMELSRLRSDDTAVYYCARGTRAMH | HC Knob 13E4v2ii |
| | YWGQGTLVTVSSASTKGPSVFPLAPSSKSTSGGTAALGCLVKDYFPEPVTVS | VH2_LALALR |
| | WNSGALTSGVHTFPAVLQSSGLYSLSSVVTVPSSSLGTQTYICNVNHKPSNT | |
| | KVDKKVEPKSCDKTHTCPPCPAPEAAGGPSVFLFPPKPKDTLMISRTPEVTC | |
| | VVVDVSHEDPEVKENWYVDGVEVHNAKTKPREEQYNSTYRVVSVLTVLHQDW | |
| | LNGKEYKCKVSNKARPAPIEKTISKAKGQPREPQVYTLPPSRDELTKNQVSL | |
| | WCLVKGFYPSDIAVEWESNGQPENNYKTTPPVLDSDGSFFLYSKLTVDKSRW | |
| | QQGNVFSCSVMHEALHNHYTQKSLSLSPGK | |
| 11 | QVQLVQSGAEVKKPGASVKVSCKASGYTFTNYWMHWVRQAPGQGLEWIGEIN | DS05290 |
| | PINGRSNYAEKFQGRVTLTVDTSSSTAYMELSRLRSDDTAVYYCARGTRAMH | HC Hole 13E4v2ii |
| | YWGQGTLVTVSSASTKGPSVFPLAPSSKSTSGGTAALGCLVKDYFPEPVTVS | VH2 LALALR S23 |
| | WNSGALTSGVHTFPAVLQSSGLYSLSSVVTVPSSSLGTQTYICNVNHKPSNT | 9C |
| | KVDKKVEPKSCQKTHTCPPCPAPEAAGGPCVFLFPPKPKDTLMISRTPEVTC | |
| | VVVDVSHEDPEVKFNWYVDGVEVHNAKTKPREEQYNSTYRVVSVLTVLHQDW | |
| | LNGKEYKCKVSNKARPAPIEKTISKAKGQPREPQVYTLPPSRDELTKNQVSL | |
| | SCAVKGFYPSDIAVEWESNGQPENNYKTTPPVLDSDGSFFLVSKLTVDKSRW | |
| | QQGNVFSCSVMHEALHNHYTQKSLSLSPGK | |
| 12 | GYTFTNYWMH | CDR-H1 |
| 13 | EINPINGRSNYAEKFQG | CDR-H2 |
| 14 | ARGTRAMHY | CDR-H3 |
| 15 | RTSENIYNNLA | CDR-L1 |
| 16 | AATNLAD | CDR-L2 |
| 17 | QHFWGTPLT | CDR-L3 |
| | monovalent TER Fab (with RSV NBF) | |
| 18 | DIQMTQSPSSLSASVGDRVTITCRASDNLYSNLAWYQQKPGKSPKLLVYDAT | TfR light chain |
| | NLADGVPSRFSGSGSGTDYTLTISSLQPEDFATYYCQHFWGTPLTFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGEC | |
| 19 | EVQLVQSGAEVKKPGASVKVSCKASGYTFTSYWMHWVRQAPGQRLEWIGELN | TIR VH LALA |
| | PTNGRTNYIEKFKSRATLTVDKSASTAYMELSSLRSEDTAVYYCARGTRAYH | Knob |
| | YWGQGTMVTVSSASTKGPSVFPLAPSSKSTSGGTAALGCLVKDYFPEPVTVS | |
| | WNSGALTSGVHTFPAVLQSSGLYSLSSVVTVPSSSLGTQTYICNVNHKPSNT | |
| | KVDKKVEPKSCQKTHTCPPCPAPEAAGGPSVFLFPPKPKDTLMISRTPEVTC | |
| | VVVDVSHEDPEVKFNWYVDGVEVHNAKTKPREEQYNSTYRVVSVLTVLHQDW | |
| | LNGKEYKCKVSNKALPAPIEKTISKAKGQPREPQVYTLPPSRDELTKNQVSL | |
| | WCLVKGFYPSDIAVEWESNGQPENNYKTTPPVLDSDGSFFLYSKLTVDKSRW | |
| | QQGNVFSCSVMHEALHNHYTQKSLSLSPGK | |
| 20 | QVTLRESGPALVKPTQTLTLTCTFSGFSLSTSGMSVGWIRQPPGKALEWLAD | palivizumab VH |
| | IWWDDKKDYNPSLKSRLTISKDTSKNQVVLKVTNMEPADTATYYCARSMITN | anti-RSV hIgG1 |
| | WYFDVWGAGTTVTVSSASTKGPSVFPLAPSSKSTSGGTAALGCLVKDYFPEP | LALA Hole S239C |
| | VTVSWNSGALTSGVHTFPAVLQSSGLYSLSSVVTVPSSSLGTQTYICNVNHK | P001889 0 |
| | PSNTKVDKKVEPKSCDKTHTCPPCPAPEAAGGPCVELFPPKPKDTLMISRTP | |
| | EVTCVVVDVSHEDPEVKENWYVDGVEVHNAKTKPREEQYNSTYRVVSVLTVL | |
| | HQDWLNGKEYKCKVSNKALPAPIEKTISKAKGQPREPQVYTLPPSRDELTKN | |
| | QVSLSCAVKGFYPSDIAVEWESNGQPENNYKTTPPVLDSDGSFFLVSKLTVD | |
| | KSRWQQGNVFSCSVMHEALHNHYTQKSLSLSPGK | |
| 21 | GYTFTSYWMH | CDR-H1 |
| 22 | EINPTNGRTNYIEKFKS | CDR-H2 |
| 23 | ARGTRAYHY | CDR-H3 |
| 24 | RASDNLYSNLA | CDR-L1 |
| 25 | DATNLAD | CDR-L2 |
| 26 | QHFWGTPLT | CDR-L3 |
| 27 | PCPAPELLGGPSVFLFPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALPAP | hole mutations |
| | IEKTISKAKGQPREPQVYTLPPSRDELTKNQVSLSCAVKGFYPSDIAVEWES | |
| | NGQPENNYKTTPPVLDSDGSFFLVSKLTVDKSRWQQGNVFSCSVMHEALHNH | |
| | YTQKSLSLSPGK | |
| 28 | PCPAPEAAGGPSVFLFPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALPAP | hole and LALA |
| | IEKTISKAKGQPREPQVYTLPPSRDELTKNQVSLSCAVKGFYPSDIAVEWES | mutations |
| | NGQPENNYKTTPPVLDSDGSPELVSKLTVDKSRWQQGNVFSCSVMHEALHNH | |
| | YTQKSLSLSPGK | |
| 29 | PCPAPEAAGGPSVFLFPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALGAP | hole and LALAPG |
| | IEKTISKAKGQPREPQVYTLPPSRDELTKNQVSLSCAVKGFYPSDIAVEWES | mutations |
| | NGQPENNYKTTPPVLDSDGSFFLVSKLTVDKSRWQQGNVFSCSVMHEALHNH | |
| | YTQKSLSLSPGK | |
| 30 | PCPAPEAAGGPSVFLFPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALSAP | hole and LALAPS |
| | IEKTISKAKGQPREPQVYTLPPSRDELTKNQVSLSCAVKGFYPSDIAVEWES | mutations |
| | NGQPENNYKTTPPVLDSDGSFFLVSKLTVDKSRWQQGNVFSCSVMHEALHNH | |
| | YTQKSLSLSPGK | |
| 31 | PCPAPEAAGGPSVFLFPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALPAP | hole and LALA |
| | IEKTISKAKGQPREPQVYTLPPSRDELTKNQVSLSCAVKGFYPSDIAVEWES | mutations |
| | NGQPENNYKTTPPVLDSDGSFFLVSKLTVDKSRWQQGNVFSCSVMHEALHNH | |
| | YTQKSLSLSPG | |
| 32 | PCPAPEAAGGPSVFLFPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALGAP | hole and LALAPG |
| | IEKTISKAKGQPREPQVYTLPPSRDELTKNQVSLSCAVKGFYPSDIAVEWES | mutations |
| | NGQPENNYKTTPPVLDSDGSFFLVSKLTVDKSRWQQGNVFSCSVMHEALHNH | |
| | YTQKSLSLSPG | |
| 33 | PCPAPEAAGGPSVELEPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALSAP | hole and LALAPS |
| | IEKTISKAKGQPREPQVYTLPPSRDELTKNQVSLSCAVKGFYPSDIAVEWES | mutations |
| | NGQPENNYKTTPPVLDSDGSFFLVSKLTVDKSRWQQGNVFSCSVMHEALHNH | |
| | YTQKSLSLSPG | |
| 34 | PCPAPEAAGGPSVFLFPPKPKDTLMISRTPEVTCVVVDVSHEDPEVKENWYV | Fc sequence with |
| | DGVEVHNAKTKPREEQYNSTYRVVSVLTVLHQDWLNGKEYKCXVSNKALPAP | knob and LALA |
| | IEKTISKAKGQPREPQVYTLPPSRDELTKNQVSLWCLVKGFYPSDIAVEWES | mutations |
| | NGQPENNYKTTPPVLDSDGSFFLYSKLTVDKSRWQQGNVFSCSVMHEALHNH | |
| | YTQKSLSLSPGK | |
| 35 | EIVMTQSPATLSLSPGERATLSCSASSSVSYMYWYQQKPGQAPRLLIYDTSN | NBVR1 Light chain |
| | LASGIPARFSGSGSGTDFTLTISSLQPEDFAVYYCQQWSSYPPITFGQGTKV | variable domain |
| | EIK | |
| 36 | QVQLQESGPGLVKPSETLSLTCAVSGYSITSDYAWNWIRQPPGKGLEWIGYM | NBVRI heavy chain |
| | SYSGSTRYNPSLRSRVTISVDTSKNQFSLKLSSVTAADTAVYYCARGWPLAY | variable domain |
| | WGQGTLVTVSS | |
| 37 | EIVMTQSPATLSLSPGERATLSCSASSSVSYMYWYQQKPGQAPRLLIYDTSN | NBVR1 light chain |
| | LASGIPARFSGSGSGTDFTLTISSLQPEDFAVYYCQQWSSYPPITFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVIKS | |
| | FNRGEC | |
| 38 | QVQLQESGPGLVKPSETLSLTCAVSGYSITSDYAWNWIRQPPGKGLEWIGYM | NBVRI heavy chain |
| | SYSGSTRYNPSLRSRVTISVDTSKNQFSLKLSSVTAADTAVYYCARGWPLAY | Fab |
| | WGQGTLVTVSSASTKGPSVFPLAPSSKSTSGGTAALGCLVKDYFPEPVTVSW | |
| | NSGALTSGVHTFPAVLQSSGLYSLSSVVTVPSSSLGTQTYICNVNHKPSNTK | |
| | VDKKVEPK | |
| 39 | SASSSVSMYWY | NBVR1 CDR-L1 |
| 40 | RASQSVSSYLAWY | NBVR2 CDR-L1 |
| 41 | DTSNLAS | NBVRI CDR-L2 |
| 42 | DASNLAT | NBVR2 CDR-L2 |
| 43 | QQWSSYPPIT | NBVR1 CDR-L3 |
| 44 | QQRSNWPPIT | NBVR2 CDR-L3 |
| 45 | GYSITSDYAWN | NBVRI CDR-H1 |
| 46 | GYSITSDYAWG | NBVR2 CDR-H1 |
| 47 | YMSYSGSTRYNPSLRS | NBVRI CDR-H2 |
| 48 | SMSYSGSTYYNPSLKS | NBVR2 CDR-H2 |
| 49 | ARGWPLAY | NBVR1/2 CDR-H3 |
| 50 | X₁MSYSGSTX₂YNPSLX₃S | NBVR CDR-H2 |
| | Wherein: X₁ =Y or S; X₂ = R or Y; and X₃ = R or K | consensus |
| 51 | EIVLTQSPATLSLSPGERATLSCRASQSVSSYLAWYQQKPGQAPRLLIYDAS | NB VR2 light chain |
| | NLATGIPARFSGSGSGTDFTLTISSLEPEDFAVYYCQQRSNWPPITFGQGTK | variable domain |
| | VEIK | |
| 52 | EIVMTQSPATLSLSPGERATLSCSASSSVSYMYWYQQKPGQAPRLLIYDTSN | NBVRI K149C light |
| | LASGIPARFSGSGSGTDFTLTISSLQPEDFAVYYCQQWSSYPPITFGQGTKV | chain |
| | EIKRTVAAPSVFIFPPSDEQLKSGTASVVCLLNNFYPREAKVQWCVDNALQS | |
| | GNSQESVTEQDSKDSTYSLSSTLTLSKADYEKHKVYACEVTHQGLSSPVTKS | |
| | FNRGEC | |
| 53 | QVQLQESGPGLVKPSETLSLTCAVSGYSITSDYAWNWIRQPPGKGLEWIGYM | NBVR1 heavy chain |
| | SYSGSTRYNPSLRSRVTISVDTSKNQFSLKLSSVTAADTAVYYCARGWPLAY | Fab |
| | WGQGTLVTVSSASTKGPSVFPLAPSSKSTSGGTAALGCLVKDYFPEPVTVSW | |
| | NSGALTSGVHTEPAVLQSSGLYSLSSVVTVPSSSLGTQTYICNVNHKPSNTK | |
| | VDKKVEPKSC | |
| 54 | QVQLQESGPGLVKPSETLSLTCAVSGYSITSDYAWNWIRQPPGKGLEWIGYM | NBVRI heavy chain |
| | SYSGSTRYNPSLRSRVTISVDTSKNQFSLKLSSVTAADTAVYYCARGWPLAY | Fab |
| | WGQGTLVTVSSASTKGPSVFPLAPSSKSTSGGTAALGCLVKDYFPEPVTVSW | |
| | NSGALTSGVHTEPAVLQSSGLYSLSSVVTVPSSSLGTQTYICNVNHKPSNTK | |
| | VDKKVEPKSCDKTHTCP | |
| 55 | QVQLQESGPGLVKPSETLSLTCAVSGYSITSDYAWNWIRQPPGKGLEWIGYM | NBVRI heavy chain |
| | SYSGSTRYNPSLRSRVTISVDTSKNQFSLKLSSVTAADTAVYYCARGWPLAY | Fab' |
| | WGQGTLVTVSSASTKGPSVFPLAPSSKSTSGGTAALGCLVKDYFPEPVTVSW | |
| | NSGALTSGVHTFPAVLQSSGLYSLSSVVTVPSSSLGTQTYICNVNHKPSNTK | |
| | VDKKVEPKSCDKTHTCPPCP | |
| 56 | QVQLQESGPGLVKPSETLSLTCAVSGYSITSDYAWNWIRQPPGKGLEWIGYM | NBVR1 A114C |
| | SYSGSTRYNPSLRSRVTISVDTSKNQFSLKLSSVTAADTAVYYCARGWPLAY | heavy chain Fab |
| | WGQGTLVTVSSCSTKGPSVFPLAPSSKSTSGGTAALGCLVKDYFPEPVTVSW | |
| | NSGALTSGVHTEPAVLQSSGLYSLSSVVTVPSSSLGTQTYICNVNHKPSNTK | |
| | VDKKVEPK | |
| 57 | QVQLQESGPGLVKPSETLSLTCAVSGYSITSDYAWNWIRQPPGKGLEWIGYM | NBVR1 A114C |
| | SYSGSTRYNPSLRSRVTISVDTSKNQFSLKLSSVTAADTAVYYCARGWPLAY | heavy chain Fab |
| | WGQGTLVTVSSCSTKGPSVFPLAPSSKSTSGGTAALGCLVKDYFPEPVTVSW | |
| | NSGALTSGVHTFPAVLQSSGLYSLSSVVTVPSSSLGTQTYICNVNHKPSNTK | |
| | VDKKVEPKSC | |
| 58 | QVQLQESGPGLVKPSETLSLTCAVSGYSITSDYAWNWIRQPPGKGLEWIGYM | NBVR1 A114C |
| | SYSGSTRYNPSLRSRVTISVDTSKNQFSLKLSSVTAADTAVYYCARGWPLAY | heavy chain Fab |
| | WGQGTLVTVSSCSTKGPSVFPLAPSSKSTSGGTAALGCLVKDYFPEPVTVSW | |
| | NSGALTSGVHTFPAVLQSSGLYSLSSVVTVPSSSLGTQTYICNVNHKPSNTK | |
| | VDKKVEPKSCDKTHTCP | |
| 59 | QVQLQESGPGLVKPSETLSLTCAVSGYSITSDYAWNWIRQPPGKGLEWIGYM | NBVR1 heavy chain |
| | SYSGSTRYNPSLRSRVTISVDTSKNQFSLKLSSVTAADTAVYYCARGWPLAY | Fab' |
| | WGQGTLVTVSSCSTKGPSVFPLAPSSKSTSGGTAALGCLVKDYFPEPVTVSW | |
| | NSGALTSGVHTFPAVLQSSGLYSLSSVVTVPSSSLGTQTYICNVNHKPSNTK | |
| | VDKKVEPKSCDKTHTCPPCP | |
| 60 | QVQLQESGPGLVKPSETLSLTCAVSGYSITSDYAWGWIRQPPGKGLEWIGSM | NBVR2 heavy chain |
| | SYSGSTYYNPSLKSRVTISVDTSKNQFSLKLSSVTAADTAVYYCARGWPLAY | variable domain |
| | WGQGTLVTVSS | |
| 61 | QVQLQESGPGLVKPSETLSLTCAVSGYSITSDYAWGWIRQPPGKGLEWIGSM | NBVR2 heavy chain |
| | SYSGSTYYNPSLKSRVTISVDTSKNQFSLKLSSVTAADTAVYYCARGWPLAY | Fab |
| | WGQGTLVTVSSASTKGPSVFPLAPSSKSTSGGTAALGCLVKDYFPEPVTVSW | |
| | NSGALTSGVHTFPAVLQSSGLYSLSSVVTVPSSSLGTQTYICNVNHKPSNTK | |
| | VDKKVEPK | |
| 62 | EIVLTQSPATLSLSPGERATLSCRASQSVSSYLAWYQQKPGQAPRLLIYDAS | NB VR2 light chain |
| | NLATGIPARFSGSGSGTDFTLTISSLEPEDFAVYYCQQRSNWPPITFGQGTK | |
| | VEIKRTVAAPSVFIFPPSDEQLKSGTASVVCLLNNFYPREAKVQWKVDNALQ | |
| | SGNSQESVTEQDSKDSTYSLSSTLTLSKADYEKHKVYACEVTHQGLSSPVTK | |
| | SENRGEC | |
| 63 | EIVLTQSPATLSLSPGERATLSCRASQSVSSYLAWYQQKPGQAPRLLLYDAS | NB VR2 light chain |
| | NLATGIPARFSGSGSGTDFTLTISSLEPEDFAVYYCQQRSNWPPITEGQGTK | variable domain |
| | VEIK | |
| 64 | EIVLTQSPATLSLSPGERATLSCRASQSVSSYLAWYQQKPGQAPRLLIYDAS | NBVR2 K149C light |
| | NLATGIPARFSGSGSGTDFTLTISSLEPEDFAVYYCQQRSNWPPITFGQGTK | chain |
| | VEIKRTVAAPSVFIFPPSDEQLKSGTASVVCLLNNFYPREAKVQWCVDNALQ | |
| | SGNSQESVTEQDSKDSTYSLSSTLTLSKADYEKHKVYACEVTHQGLSSPVTK | |
| | SENRGEC | |
| 65 | QVQLQESGPGLVKPSETLSLTCAVSGYSITSDYAWGWIRQPPGKGLEWIGSM | NBVR2 heavy chain |
| | SYSGSTYYNPSLKSRVTISVDTSKNQFSLKLSSVTAADTAVYYCARGWPLAY | Fab |
| | WGQGTLVTVSSASTKGPSVFPLAPSSKSTSGGTAALGCLVKDYFPEPVTVSW | |
| | NSGALTSGVHTFPAVLQSSGLYSLSSVVTVPSSSLGTQTYICNVNHKPSNTK | |
| | VDKKVEPKSC | |
| 66 | QVQLQESGPGLVKPSETLSLTCAVSGYSITSDYAWGWIRQPPGKGLEWIGSM | NBVR2 heavy chain |
| | SYSGSTYYNPSLKSRVTISVDTSKNQFSLKLSSVTAADTAVYYCARGWPLAY | Fab |
| | WGQGTLVTVSSASTKGPSVFPLAPSSKSTSGGTAALGCLVKDYFPEPVTVSW | |
| | NSGALTSGVHTEPAVLQSSGLYSLSSVVTVPSSSLGTQTYICNVNHKPSNTK | |
| | VDKKVEPKSCDKTHTCP | |
| 67 | QVQLQESGPGLVKPSETLSLTCAVSGYSITSDYAWGWIRQPPGKGLEWIGSM | NBVR2 heavy chain |
| | SYSGSTYYNPSLKSRVTISVDTSKNQFSLKLSSVTAADTAVYYCARGWPLAY | Fab |
| | WGQGTLVTVSSASTKGPSVFPLAPSSKSTSGGTAALGCLVKDYFPEPVTVSW | |
| | NSGALTSGVHTFPAVLQSSGLYSLSSVVTVPSSSLGTQTYICNVNHKPSNTK | |
| | VDKKVEPKSCDKTHTCPPCP | |
| 68 | QVQLQESGPGLVKPSETLSLTCAVSGYSITSDYAWGWIRQPPGKGLEWIGSM | NBVR2 A114C |
| | SYSGSTYYNPSLKSRVTISVDTSKNQFSLKLSSVTAADTAVYYCARGWPLAY | heavy chain Fab |
| | WGQGTLVTVSSCSTKGPSVFPLAPSSKSTSGGTAALGCLVKDYFPEPVTVSW | |
| | NSGALTSGVHTFPAVLQSSGLYSLSSVVTVPSSSLGTQTYICNVNHKPSNTK | |
| | VDKKVEPK | |
| 69 | QVQLQESGPGLVKPSETLSLTCAVSGYSITSDYAWGWIRQPPGKGLEWIGSM | NBVR2 A114C |
| | SYSGSTYYNPSLKSRVTISVDTSKNQFSLKLSSVTAADTAVYYCARGWPLAY | heavy chain Fab |
| | WGQGTLVTVSSCSTKGPSVFPLAPSSKSTSGGTAALGCLVKDYFPEPVTVSW | |
| | NSGALTSGVHTFPAVLQSSGLYSLSSVVTVPSSSLGTQTYICNVNHKPSNTK | |
| | VDKKVEPKSC | |
| 70 | QVQLQESGPGLVKPSETLSLTCAVSGYSITSDYAWGWIRQPPGKGLEWIGSM | NBVR2 A114C |
| | SYSGSTYYNPSLKSRVTISVDTSKNQFSLKLSSVTAADTAVYYCARGWPLAY | heavy chain Fab |
| | WGQGTLVTVSSCSTKGPSVFPLAPSSKSTSGGTAALGCLVKDYFPEPVTVSW | |
| | NSGALTSGVHTFPAVLQSSGLYSLSSVVTVPSSSLGTQTYICNVNHKPSNTK | |
| | VDKKVEPKSCDKTHTCP | |
| 71 | QVQLQESGPGLVKPSETLSLTCAVSGYSITSDYAWGWIRQPPGKGLEWIGSM | NBVR2 A114C |
| | SYSGSTYYNPSLKSRVTISVDTSKNQFSLKLSSVTAADTAVYYCARGWPLAY | heavy chain Fab' |
| | WGQGTLVTVSSCSTKGPSVFPLAPSSKSTSGGTAALGCLVKDYFPEPVTVSW | |
| | NSGALTSGVHTFPAVLQSSGLYSLSSVVTVPSSSLGTQTYICNVNHKPSNTK | |
| | VDKKVEPKSCDKTHTCPPCP | |
| 72 | gaaattgtcctcacccagagcccagccactctttccctctctccaggggaaa | NB VR2 light chain |
| | gagcaacactgagttgccgggcctcccagagtgtaagttcctacctcgcatg | variable domain |
| | gtatcaacagaagcctggtcaagcaccccggcttctgatttacgatgccagc | coding sequence |
| | aatctcgccaccggaatccccgcaaggttttcagggagcggttcagggactg | |
| | actttaccctgaccataagcagtcttgagcctgaggattttgcagtgtatta | |
| | ctgccaacagagaagtaactggccccccataacctttggacaggggactaag | |
| | gtggagattaaa | |
| 73 | gagatcgtactcactcaaagtccagccaccctgtcactttctccaggagaac | NBVR2 light chain |
| | gcgccactctctcttgtcgggctagtcagagcgttagtagctatctcgcatg | variable domain |
| | gtatcaacaaaagcccggtcaggccccacgcctgttgatctatgatgccagt | coding sequence |
| | aacctggccaccggtattccagcccgtt:ctctggcagtggaagtggaactg | |
| | acttcacactgaccatcagctccctcgagcccgaagattttgctgtgtatta | |
| | ctgccagcaaagatccaactggcctccaatcactttcggccaaggaacaaaa | |
| | gtagagattaag | |
| 74 | caagtgcaactgcaggaaagtgggcctggtttggtcaaacccagtgaaacac | NBVR2 heavy chain |
| | ttagtcttacttqcgctgtatctggatactctattacaagcgattatgcctg | variable domain |
| | ggggtggattcggcagccccctggaaaaggcttggagtggatcggttcaatg | coding sequence |
| | agttatagtggtagtacatactacaacccttccctgaagtctcgcgtcacaa | |
| | taagcgtagatacaagtaagaatcaattttccctgaaacttagcagtgtaac | |
| | tgccgctgataccgcagtctactattgtgccagaggatggcccctggcctac | |
| | tggggtcagggcaccctcgtgaccgtatcatca | |
| 75 | caggttcaacttcaagagtcaggcccaggcttggtgaagccctccgaaactt | NBVR2 heavy chain |
| | tgagcttgacttgcgctgtgtccggttacagtataacctctgactatgcctg | variable domain |
| | ggggtggataaggcaacctcctggcaaaggattggaatggatcggttcaatg | coding sequence |
| | tcctactccggttctacatactataacccctccttgaagtcccgagtaacta | |
| | tcagtgtggacacttcaaagaatcaatt:agtctgaaattgtcttcagtcac | |
| | cgctgcagacactgctgtctattattgtgccaggggttggccccttgcttat | |
| | tggggccaggggaccctggtgacagtatcaagc | |
| 76 | PCPAPELLGGPSVFLFPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALPAP | hole mutations |
| | IEKTISKAKGQPREPQVYTLPPSRDELTKNQVSLSCAVKGFYPSDIAVEWES | |
| | NGQPENNYKTTPPVLDSDGSFFLVSKLTVDKSRWQQGNVFSCSVMHEALHNH | |
| | YTQKSLSLSPGK | |
| 77 | PCPAPEAAGGPSVELEPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALPAP | hole and LALA |
| | IEKTISKAKGQPREPQVYTLPPSRDELTKNQVSLSCAVKGFYPSDIAVEWES | mutations |
| | NGQPENNYKTTPPVLDSDGSFFLVSKLTVDKSRWQQGNVFSCSVMHEALHNH | |
| | YTQKSLSLSPGK | |
| 78 | PCPAPEAAGGPCVFLFPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALPAP | hole, LALA, and |
| | IEKTISKAKGQPREPQVYTLPPSRDELTKNQVSLSCAVKGFYPSDIAVEWES | S239C mutations |
| | NGQPENNYKTTPPVLDSDGSFFLVSKLTVDKSRWQQGNVFSCSVMHEALHNH | |
| | YTQKSLSLSPGK | |
| 79 | PCPAPEAAGGPSVFLFPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALPCP | hole, LALA and |
| | IEKTISKAKGQPREPQVYTLPPSRDELTKNQVSLSCAVKGFYPSDIAVEWES | A330C mutations |
| | NGQPENNYKTTPPVLDSDGSFFLVSKLTVDKSRWQQGNVFSCSVMHEALHNH | |
| | YTQKSLSLSPGK | |
| 80 | PCPAPEAAGGPSVFLFPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALPAP | hole, LALA and |
| | IEKTISKAKGQPREPQVYTLPPSRDELTKNQVSLSCAVKGFYPSDIAVEWES | S442C mutations |
| | NGQPENNYKTTPPVLDSDGSFFLVSKLTVDKSRWQQGNVFSCSVMHEALHNH | |
| | YTQKSLCLSPGK | |
| 81 | PCPAPEAAGGPSVELFPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALGAP | hole and LALAPG |
| | IEKTISKAKGQPREPQVYTLPPSRDELTKNQVSLSCAVKGFYPSDIAVEWES | mutations |
| | NGQPENNYKTTPPVLDSDGSFFLVSKLTVDKSRWQQGNVFSCSVMHEALHNH | |
| | YTQKSLSLSPGK | |
| 82 | PCPAPEAAGGPSVFLFPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALSAP | hole and LALAPS |
| | IEKTISKAKGQPREPQVYTLPPSRDELTKNQVSLSCAVKGFYPSDIAVEWES | mutations |
| | NGQPENNYKTTPPVLDSDGSFFLVSKLTVDKSRWQQGNVFSCSVMHEALHNH | |
| | YTQKSLSLSPGK | |
| 83 | PCPAPEAAGGPCVFLFPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALSAP | hole, LALAPS, and |
| | IEKTISKAKGQPREPQVYTLPPSRDELTKNQVSLSCAVKGFYPSDIAVEWES | S239C mutations |
| | NGQPENNYKTTPPVLDSDGSFFLVSKLTVDKSRWQQGNVFSCSVMHEALHNH | |
| | YTQKSLSLSPGK | |
| 84 | PCPAPEAAGGPSVFLFPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALSCP | hole, LALAPS and |
| | IEKTISKAKGQPREPQVYTLPPSRDELTKNQVSLSCAVKGFYPSDIAVEWES | A330C mutations |
| | NGQPENNYKTTPPVLDSDGSFFLVSKLTVDKSRWQQGNVFSCSVMHEALHNH | |
| | YTQKSLSLSPGA | |
| 85 | PCPAPEAAGGPSVFLFPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALSAP | hole, LALAPS and |
| | IEKTISKAKGQPREPQVYTLPPSRDELTKNQVSLSCAVKGFYPSDIAVEWES | S442C mutations |
| | NGQPENNYKTTPPVLDSDGSFFLVSKLTVDKSRWQQGNVFSCSVMHEALHNH | |
| | YTQKSLCLSPGK | |
| 86 | PCPAPEAAGGPSVFLFPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALPAP | knob and LALA |
| | IEKTISKAKGQPREPQVYTLPPSRDELTKNQVSLWCLVKGFYPSDIAVEWES | mutations |
| | NGQPENNYKTTPPVLDSDGSFFLYSKLTVDKSRWQQGNVFSCSVMHEALHNH | |
| | YTQKSLSLSPGK | |
| 87 | PCPAPEAAGGPSVFLFPPKPKDTLMISRTPEVTCVVVDVSHEDPEVKENWYV | Fc sequence with |
| | DGVEVHNAKCKPREEQYNSTYRVVSVLTVLHQDWLNGKEYKCKVSNKALPAP | hole, LALA, and |
| | IEKTISKAKGQPREPQVYTLPPSRDELTKNQVSLSCAVKGFYPSDIAVEWES | T289C mutations |
| | NGQPENNYKTTPPVLDSDGSFFLVSKLTVDKSRWQQGNVFSCSVMHEALHNH | |
| | YTQKSLSLSPGK | |
| 88 | PCPAPEAAGGPSVELEPPKPKDTLMISRTPEVTCVVVDVSHEDPEVKENWYV | Fc sequence with |
| | DGVEVHNAKTKPREEQYASTYRVVSVLTVLHQDWLNGKEYKCKVSNKALPAP | hole, LALA, and |
| | IEKTISKAKGQPREPQVYTLPPSRDELTKNQVSLSCAVKGFYPSDIAVEWES | N297A mutations |
| | NGQPENNYKTTPPVLDSDGSFFLVSKLTVDKSRWQQGNVFSCSVMHEALHNH | |
| | YTQKSLSLSPGK | |
| 89 | PCPAPEAAGGPSVFLFPPKPKDTLMISRTPEVTCVVVDVSHEDPEVKENWYV | Fc sequence with |
| | DGVEVHNAKTKPREEQYGSTYRVVSVLTVLHQDWLNGKEYKCKVSNKALPAP | hole, LALA, and |
| | IEKTISKAKGQPREPQVYTLPPSRDELTKNQVSLSCAVKGFYPSDIAVEWES | N297G mutations |
| | NGQPENNYKTTPPVLDSDGSFFLVSKLTVDKSRWQQGNVFSCSVMHEALHNH | |
| | YTQKSLSLSPGK | |
| 90 | PCPAPEAAGGPCVFLFPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALPCP | hole, LALA, S239C, |
| | IEKTISKAKGQPREPQVYTLPPSRDELTKNQVSLSCAVKGFYPSDIAVEWES | and A330C |
| | NGQPENNYKTTPPVLDSDGSFFLVSKLTVDKSRWQQGNVFSCSVMHEALHNH | mutations |
| | YTQKSLSLSPGK | |
| 91 | CSTKGPSVFPLAPSSKSTSGGTAALGCLVKDYFPEPVTVSWNSGALTSGVHT | Heavy constant |
| | FPAVLQSSGLYSLSSVVTVPSSSLGTQTYICNVNHKPSNTKVDKKVEPKSCD | region sequence with |
| | KTHTCPPCPAPEAAGGPSVELFPPKPKDTLMISRTPEVTCVVVDVSHEDPEV | hole, LALA, and |
| | KFNWYVDGVEVHNAKTKPREEQYNSTYRVVSVLTVLHQDWLNGKEYKCKVSN | A114C mutations |
| | KALPAPIEKTISKAKGQPREPQVYTLPPSRDELTKNQVSLSCAVKGFYPSDI | |
| | AVEWESNGQPENNYKTTPPVLDSDGSFFLVSKLTVDKSRWQQGNVFSCSVMH | |
| | EALHNHYTQKSLSLSPGK | |
| 92 | PCPAPEAAGGPCVFLFPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALGAP | hole, LALAPG, and |
| | IEKTISKAKGQPREPQVYTLPPSRDELTKNQVSLSCAVKGFYPSDIAVEWES | S239C mutations |
| | NGQPENNYKTTPPVLDSDGSFFLVSKLTVDKSRWQQGNVFSCSVMHEALHNH | |
| | YTQKSLSLSPGK | |
| 93 | PCPAPEAAGGPSVFLFPPKPKDTLMISRTPEVTCVVVDVSHEDPEVKENWYV | Fc sequence with |
| | DGVEVHNAKCKPREEQYNSTYRVVSVLTVLHQDWLNGKEYKCKVSNKALGAP | hole and LALAPG, |
| | IEKTISKAKGQPREPQVYTLPPSRDELTKNQVSLSCAVKGFYPSDIAVEWES | and T289C |
| | NGQPENNYKTTPPVLDSDGSFFLVSKLTVDKSRWQQGNVFSCSVMHEALHNH | mutations |
| | YTQKSLSLSPGK | |
| 94 | PCPAPEAAGGPSVFLFPPKPKDTLMISRTPEVTCVVVDVSHEDPEVKENWYV | Fc sequence with |
| | DGVEVHNAKTKPREEQYGSTYRVVSVLTVLHQDWLNGKEYKCKVSNKALGAP | hole and LALAPG, |
| | IEKTISKAKGQPREPQVYTLPPSRDELTKNQVSLSCAVKGFYPSDIAVEWES | and N297G |
| | NGQPENNYKTTPPVLDSDGSFFLVSKLTVDKSRWQQGNVFSCSVMHEALHNH | mutations |
| | YTQKSLSLSPGK | |
| 95 | PCPAPEAAGGPSVFLFPPKPKDTLMISRTPEVTCVVVDVSHEDPEVKENWYV | Fc sequence with |
| | DGVEVHNAKTKPREEQYASTYRVVSVLTVLHQDWLNGKEYKCKVSNKALGAP | hole and LALAPG, |
| | LEKTISKAKGQPREPQVYTLPPSRDELTKNQVSLSCAVKGEYPSDLAVEWES | and N297A |
| | NGQPENNYKTTPPVLDSDGSFFLVSKLTVDKSRWQQGNVFSCSVMHEALHNH | mutations |
| | YTQKSLSLSPGX | |
| 96 | PCPAPEAAGGPSVFLFPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALGCP | hole, LALAPG, and |
| | IEKTISKAKGQPREPQVYTLPPSRDELTKNQVSLSCAVKGFYPSDIAVEWES | A330C mutations |
| | NGQPENNYKTTPPVLDSDGSFFLVSKLTVDKSRWQQGNVFSCSVMHEALHNH | |
| | YTQKSLSLSPGK | |
| 97 | PCPAPEAAGGPSVFLFPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALGAP | hole, LALAPG, and |
| | IEKTISKAKGQPREPQVYTLPPSRDELTKNQVSLSCAVKGFYPSDIAVEWES | S442C mutations |
| | NGQPENNYKTTPPVLDSDGSFFLVSKLTVDKSRWQQGNVFSCSVMHEALHNH | |
| | YTQKSLCLSPGK | |
| 98 | PCPAPEAAGGPCVFLFPPKPKDTLMISRTPEVTCVVVDVSHEDPEVKENWYV | Fc sequence with |
| | DGVEVHNAKTKPREEQYNSTYRVVSVLTVLHQDWLNGKEYKCKVSNKALGCP | hole, LALAPG, |
| | IEKTISKAKGQPREPQVYTLPPSRDELTKNQVSLSCAVKGFYPSDIAVEWES | S239C, and A330C |
| | NGQPENNYKTTPPVLDSDGSFFLVSKLTVDKSRWQQGNVFSCSVMHEALHNH | mutations |
| | YTQKSLSLSPGK | |
| 99 | CSTKGPSVEPLAPSSKSTSGGTAALGCLVKDYFPEPVTVSWNSGALTSGVHT | Heavy constant |
| | FPAVLQSSGLYSLSSVVTVPSSSLGTQTYICNVNHKPSNTKVDKKVEPKSCD | region sequence with |
| | KTHTCPPCPAPEAAGGPSVFLFPPKPKDTLMISRTPEVTCVVVDVSHEDPEV | hole, LALAPG, and |
| | KFNWYVDGVEVHNAKTKPREEQYNSTYRVVSVLTVLHQDWLNGKEYKCKVSN | A114C mutations |
| | KALGAPIEKTISKAKGQPREPQVYTLPPSRDELTKNQVSLSCAVKGFYPSDI | |
| | AVEWESNGQPENNYKTTPPVLDSDGSFFLVSKLTVDKSRWQQGNVFSCSVMH | |
| | EALHNHYTQKSLSLSPGK | |
| 100 | RTVAAPSVFIFPPSDEQLKSGTASVVCLLNNFYPREAKVQWCVDNALQSGNS | Kappa constant |
| | QESVTEQDSKDSTYSLSSTLTLSKADYEKHKVYACEVTHQGLSSPVTKSENR | region sequence with |
| | GEC | K149C mutation |
| 101 | DIQMTQSPSTLSASVGDRVTITCKCQLSVGYMHWYQQKPGKAPKLLIYDTSK | RSV Light Chain |
| | LASGVPSRESGSGSGTEFTLTISSLQPDDFATYYCFQGSGYPFTFGGGTKLE | |
| | IKRTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTKSF | |
| | NRGEC | |
| 172 | AGTACTATAGCATQTG | Malat1 ASO |
| | | (cynomolgus |
| | | monkey) |
| | monovalent TIR Fab (without RSV) | |
| 173 | DKTHTCPPCPAPEAAGGPSVFLFPPKPKDTLMISRTPEVTCVVVDVSHEDPE | Heavy Chain-WT Fc |
| | VKFNWYVDGVEVHNAKTKPREEQYNSTYRVVSVLTVLHQDWLNGKEYKCKVS | LALA knob |
| | NKALPAPIEKTISKAKGQPREPQVYTLPPSRDELTKNQVSLWCLVKGFYPSD | |
| | LAVEWESNGQPENNYKTTPPVLDSDGSFFLYSKLTVDKSRWQQGNVESCSVM | |
| | HEALHNHYTQKSLSLSPGK | |
| 174 | EVQLVQSGAEVKKPGASVKVSCKASGYTETSYWMHWVRQAPGQRLEWIGEIN | Heavy Chain-TfR |
| | PTNGRTNYIEKFKSRATLTVDKSASTAYMELSSLRSEDTAVYYCARGTRAYH | VH Hole LALA S |
| | YWGQGTMVTVSSASTKGPSVFPLAPSSKSTSGGTAALGCLVKDYFPEPVTVS | 239C |
| | WNSGALTSGVHTFPAVLQSSGLYSLSSVVTVPSSSLGTQTYICNVNHKPSNT | |
| | KVDKKVEPKSCDKTHTCPPCPAPEAAGGPCVFLFPPKPKDTLMISRTPEVTC | |
| | VVVDVSHEDPEVKENWYVDGVEVHNAKTKPREEQYNSTYRVVSVLTVLHQDW | |
| | LNGKEYKCKVSNKALPAPIEKTISKAKGQPREPQVYTLPPSRDELTKNQVSL | |
| | SCAVKGFYPSDIAVEWESNGQPENNYKTTPPVLDSDGSFFLVSKLTVDKSRW | |
| | QQGNVFSCSVMHEALHNHYTQKSLSLSPGK | |

### EXAMPLES

The subject matter will be described in further detail by way of specific examples. The following examples are offered for illustrative purposes only, and are not intended to limit the invention in any manner. Those of skill in the art will readily recognize a variety of noncritical parameters which can be changed or modified to yield essentially the same results. Efforts have been made to ensure accuracy with respect to numbers used (e.g., amounts, temperatures, etc.), but some experimental error and deviation may be present. The practice of the present invention will employ, unless otherwise indicated, conventional methods of protein chemistry, biochemistry, recombinant DNA techniques and pharmacology, within the skill of the art. Such techniques are explained fully in the literature.

### Example 1: Mono-Fab and Bivalent Antibody Conjugates

Heavy chain vectors were co-transfected to Expi293 cells along with the corresponding light chain vector in the ratio knob:hole:light chain of 1:1:2 for the mono-Fab and bivalent antibody. The expressed protein was purified from conditioned media by loading the supernatant over a Protein A column. The column was washed with 10 column volumes of PBS, pH 7.4. The proteins were eluted with 50 mM sodium citrate, pH 3.0 containing 150 mM NaCl, and immediately neutralized with 200 mM arginine, 137 mM succinic acid, pH 5.0. The proteins were further purified by size-exclusion chromatography (SEC) (GE Superdex200) using 200 mM arginine, 137 mM succinic acid, pH 5.0 as running buffer. The purified proteins were confirmed by intact mass LC/MS, and purity of >95% was confirmed by SDS-PAGE and analytical HPLC-SEC. Binding to human and cynomolgus monkey TfR apical domain was tested via biacore.

TABLE 4

| Molecule | HC1 | HC2 | LC1 | LC2 |
|---|---|---|---|---|
| TfR-mono Fab | SEQ ID | SEQ ID | SEQ ID | SEQ ID |
| | NO: 19 | NO: 20 | NO: 18 | NO: 101 |
| TfR-mono Fab 2 | SEQ ID | SEQ ID | SEQ ID | N/A |
| | NO: 173 | NO: 174 | NO: 18 | |
| Bivalent anti-TfR | SEQ ID | SEQ ID | SEQ ID | SEQ ID |
| antibody | NO: 10 | NO: 11 | NO: 9 | NO: 9 |

The mono-Fab and bivalent antibodies generated above contain a cysteine modification for conjugation and were first reduced using a reducing reagent (e.g., TCEP). Post reduction, remaining reducing agent was removed (purification by e.g. dialysis) and the antibodies are reoxidized with an oxidizing agent (e.g. dHAA). An ASO comprising a linking group was also generated, followed by a reduction and oxidation step. The reduced and oxidized linker-ASO was then conjugated to the free cysteine on the mono-Fab and bivalent antibodies. The resulting conjugate was purified to remove unwanted and unconjugated products and purity is determined by LC/MS and SEC.

An exemplary ASO sequence used herein that targets MALAT1 is: 5′-Gₖₛ^{m}CₖₛAₖₛT_{ds}T_{ds}^{m}C_{ds}T_{ds}A_{ds}A_{ds}T_{ds}A_{ds}G_{ds}^{m}C_{ds}AₖₛGₖₛ^{m}Cₖ-3′ (SEQ ID NO: 8; mouse MALAT1). The abbreviations refer to the components as follows: d: DNA; k: LNA; ^{m}C: 5-methylcytidine (methylated cytosine); s: phosphorothioate backbone (PS). The ASO is modified with a 5′ C6 amine. Another exemplary ASO sequence that targets MALAT1 is SEQ ID NO: 172 (cynomolgus monkey MALAT1)

An exemplary linking group used herein is shown below, wherein the linking group is attached to a sulfur atom of a cysteine residue within the mono-Fab or bivalent antibody and is attached to the ASO through a phosphate associated with the 5′ terminal residue of the ASO:

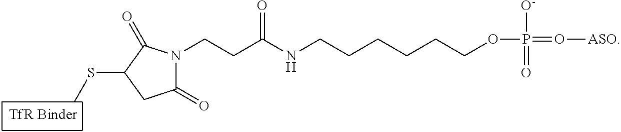

### Example 2: In Vivo Pharmacokinetics and Malat1 Knockdown Using TfR Mono-Fab Conjugate

The monovalent TfR Fab conjugate prepared above (“TfR mono-Fab”) was diluted in sterile saline before administration. As controls, saline, unconjugated ASO, and RSV-ASO groups were included.

In a single dose study, 2mo old TfR^{ms/hu} female mice were administered doses intravenously according to the groups (n=4) in Table 4 below. Tissue was collected 24 hours after the single dose. In particular, brain, spinal cord, and peripheral organs (kidney, lung, liver, and quadricep muscle) were harvested. Terminal blood was also collected 24 hours after the single dose.

In a multi-dose study, 2mo old TfR^{ms/hu} female mice were administered doses intravenously according to the groups (n=6) in Table 5 on Day 1, Day 7, and Day 14. Plasma collections were taken at 30 min, 4 hours, 24 hours, 48 hours, 72 hours, and 1 week. Tissue was collected 72 hours after the last dose. In particular, brain, spinal cord, and peripheral organs (kidney, lung, liver, and quadricep muscle) were harvested. Terminal blood was also collected 72 hours after the last dose.

TABLE 5

| Group | Description | Dose (mg/kg) |
|---|---|---|
| 1 | Neg Control: Saline | |
| 2 | Neg Control: Naked ASO (unconjugated ASO) | 1 |
| 3 | Neg Control: RSV-ASO | 25 |
| 8 | Mono-Fab | 25 |

Intact drug and total ASO were measured according to the methods described below.

huIgG Assay

Quantification of humanized antibodies in mouse plasma and tissue lysates were measured using a generic electrochemiluminescence immunoassay (ECLIA). Briefly, to the wells of an MSD GOLD 96-well streptavidin-coated microtiter plate (Meso Scale Discovery, Rockville, MD), a working concentration of biotinylated goat anti-human IgG polyclonal primary antibody (Southern Biotech, Birmingham, AL) prepared in assay diluent was incubated for approximately 1 hr. Following this incubation and a plate wash step, prepared test samples (with sample pre-dilution, where appropriate) and relevant standards were added to the assay plate and allowed to incubate for approximately 1 hr. Following test sample incubation and a plate wash step, secondary ruthenylated (SULFO-TAG) goat anti-human IgG antibody (Meso Scale Discovery, Rockville, MD) at a working concentration in assay diluent was added to the assay plate and incubated for approximately 1 hr. Following a plate wash, a 1×MSD Read Buffer T (Meso Scale Discovery, Rockville, MD) was then added to generate the electrochemiluminescence (ECL) assay signal, which was then expressed in ECL units (ECLU). All of the assay reaction steps were performed at ambient temperature with shaking on a plate shaker (where appropriate); and all test samples were pre-diluted at the assay MRD of 1:20 prior to analyzing in the assay plate. Sample ECLU signals generated in the assay subsequently were processed into concentrations by back-calculating off the assay calibration (CS) curve. The assay CS curve was fitted with a weighted four-parameter nonlinear logistic regression for use in calculating concentrations for unknown/test samples.

### Intact Drug Assay

Quantification of intact drug (anti-TfR antibody conjugated to an antisense oligonucleotide (ASO)) in mouse plasma and tissue lysates were measured using a hybridization-based electrochemiluminescence immunoassay (ECLIA). Briefly, custom biotinylated antisense probes (synthesized by Integrated DNA Technologies, Coralville, IA) at a working concentration were incubated with prepared test samples (with sample pre-dilution, where appropriate) and relevant standards in TE Buffer (10 mM Tris-HCL containing 1 mM EDTA) and hybridized at an appropriate temperature for 45 mins. Following the incubation, hybridized product was added to the wells of an MSD GOLD 96-well streptavidin-coated microtiter plate (Meso Scale Discovery, Rockville, MD) and incubated for approximately 30 mins. Following hybrid product incubation and a plate wash step, secondary ruthenylated (SULFO-TAG) goat anti-human IgG antibody (Meso Scale Discovery, Rockville, MD) at a working concentration in assay diluent was added to the assay plate and incubated for approximately 1 hr. Following a plate wash, a 1×MSD Read Buffer T (Meso Scale Discovery, Rockville, MD) was then added to generate the electrochemiluminescence (ECL) assay signal, which was then expressed in ECL units (ECLU). All of the assay reaction steps were performed at ambient temperature with shaking on a plate shaker (where appropriate); and all test samples were pre-diluted at the assay MRD of 1:20 prior to analyzing in the assay plate. Sample ECLU signals generated in the assay subsequently were processed into concentrations by back-calculating off the assay calibration (CS) curve. The assay CS curve was fitted with a weighted four-parameter nonlinear logistic regression for use in calculating concentrations for unknown/test samples.

### Total ASO Assay

Quantification of total ASO (in conjugated and free forms) in mouse plasma and tissue homogenates were measured using a hybridization-based electrochemiluminescence immunoassay (ECLIA). Briefly, custom biotinylated and digoxigenin-conjugated antisense probes (synthesized by Integrated DNA Technologies, Coralville, IA) at working concentrations were combined with prepared test samples (with sample pre-dilution, where appropriate) and relevant standards in TE Buffer (10 mM Tris-HCL containing 1 mM EDTA). Prepared samples in TE buffer were added, in a 1:1 mix, into 1×SSC Buffer (Sigma-Aldrich, St. Louis, MO) containing a working concentration of recombinant proteinase K enzyme (ThermoFisher, Waltham, MA). Hybridization/Enzyme mixture was then digested, detantured, annealed, and cooled in a thermal cycler instrument. Following hybrid product incubation, samples were added to the wells of an MSD GOLD 96-well streptavidin-coated microtiter plate (Meso Scale Discovery, Rockville, MD) and incubated for approximately 30 mins. Following incubation and a plate wash step, secondary rutheynlated (SULFO-TAG) sheep anti-digoxigenin antibody (Novus Biologicals, Littleton, CO) at a working concentration in assay diluent was added to the plate and incubated for approximately 30 mins. Following a plate wash, a 1×MSD Read Buffer T (Meso Scale Discovery, Rockville, MD) was then added to generate the electrochemiluminescence (ECL) assay signal, which was then expressed in ECL units (ECLU). All of the assay reaction steps were performed at ambient temperature with shaking on a plate shaker (where appropriate); and all test samples were pre-diluted at the assay MRD of 1:20 prior to analyzing in the assay plate. Sample ECLU signals generated in the assay subsequently were processed into concentrations by back-calculating off the assay calibration (CS) curve. The assay CS curve was fitted with a weighted four-parameter nonlinear logistic regression for use in calculating concentrations for unknown/test samples.

### Malat 1 Expression Assay

Malat1 expression was measured in the brain, spinal cord, liver, heart, quadricep, diaphragm, and sciatic nerve as follows. A <50 mg piece of tissue was homogenized with a bead homogenizer in Trizol for bulk RNA isolation. Homogenized tissues were incubated with chloroform for 3-5 minutes to allow for phase separation after centrifugation. The aqueous phase was then incubated with isopropanol for 10 minutes to allow for RNA precipitation followed by a 75% ethanol wash and resuspension in nuclease-free water. Malat1 expression was then measured by qPCR using the Express One-Step Superscript Kit and normalized to expression of the housekeeping gene Gapdh.

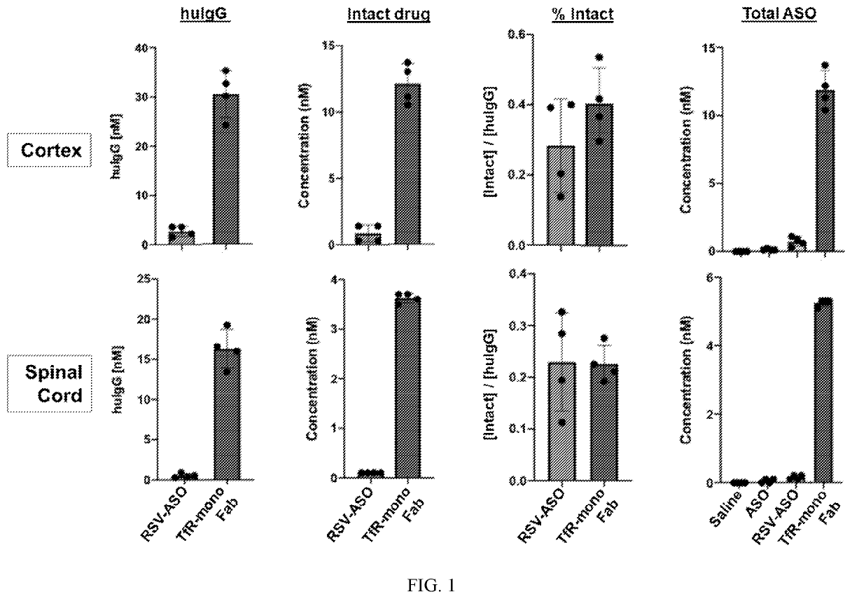

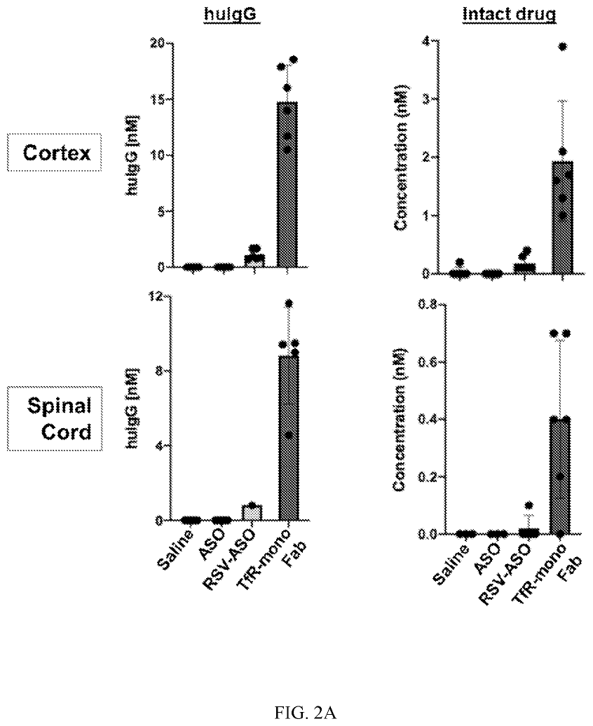

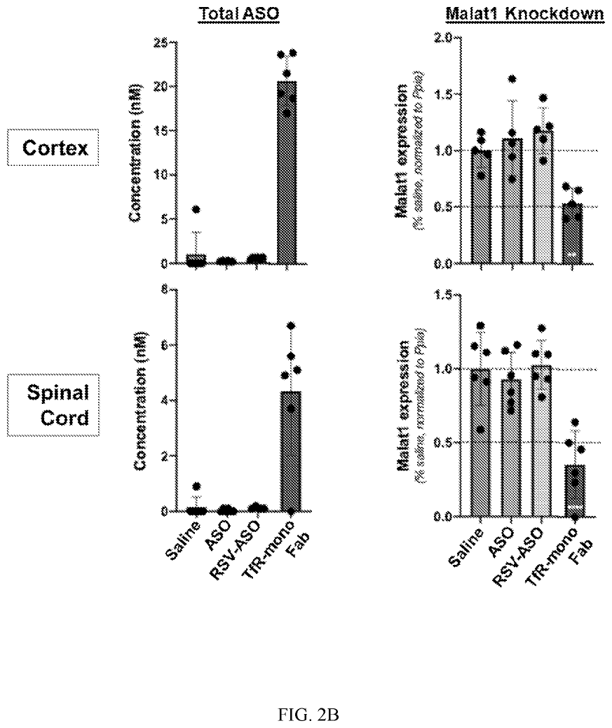

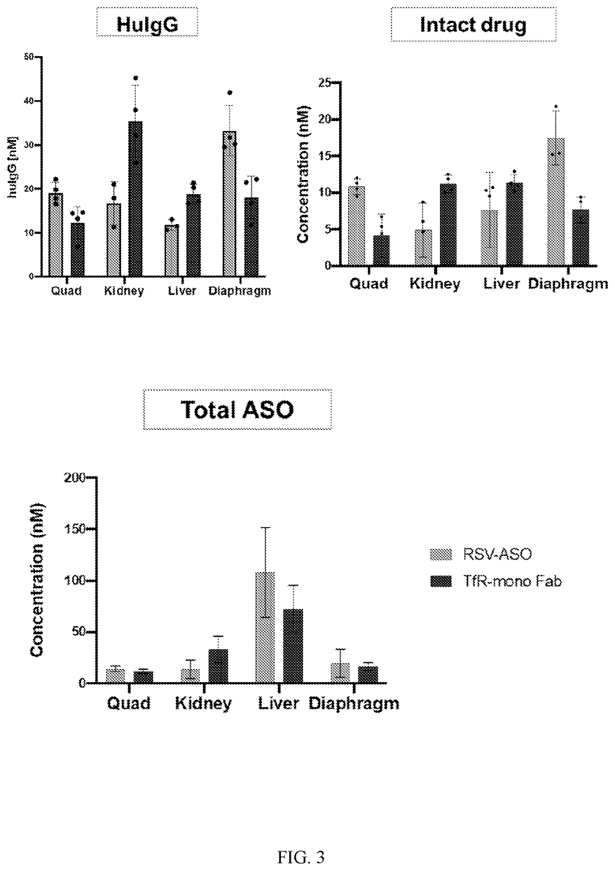

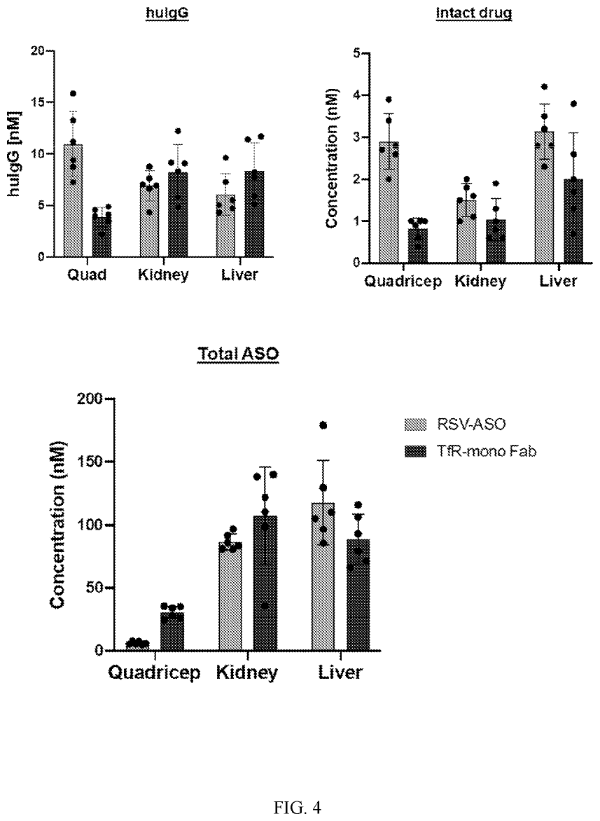

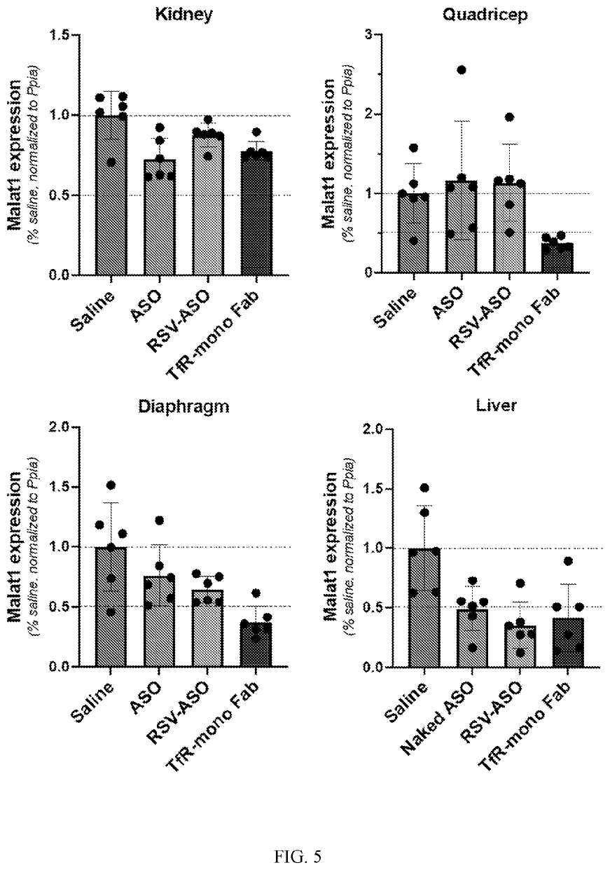

Results are shown in FIGS. 1-5. Increased delivery of intact drug and total ASO to the CNS is observed with the TfR mono-Fab conjugate compared to naked ASO and RSV-ASO controls (FIGS. 1 and 2). Increased Malat1 knockdown in the CNS is also observed compared with controls in both the single and multi-dose study (FIG. 2). Malat1 knockdown is also observed in peripheral tissue (FIG. 5). The liver is a sink for ASO 24 hours after dosing in the single dose study (FIG. 3). ASO accumulation in the liver and kidney is observed 72 hours after the final dose in the multi-dose study (FIG. 4).

### Example 3: In Vivo Malat 1 Knockdown Using Anti-TfR Bivalent Antibody Conjugate

The bivalent anti-TfR antibody conjugated to Malat1 ASO as prepared in Example 1 was diluted in sterile saline and administered to TfR^{ms/hu} knock-in mice intravenously at a weekly dose of 50 mg/kg for 4 weeks. Two control groups of TfR^{ms/hu} mice were dosed with either sterile saline or unconjugated ASO intravenously. Three days after the fourth dose, tissues were collected and frozen for molecular and biochemical analysis. Tissues include the brain, spinal cord, liver, heart, quadricep, diaphragm, and sciatic nerve.

Malat1 expression was measured in the brain, spinal cord, liver, heart, quadricep, diaphragm, and sciatic nerve as described above.

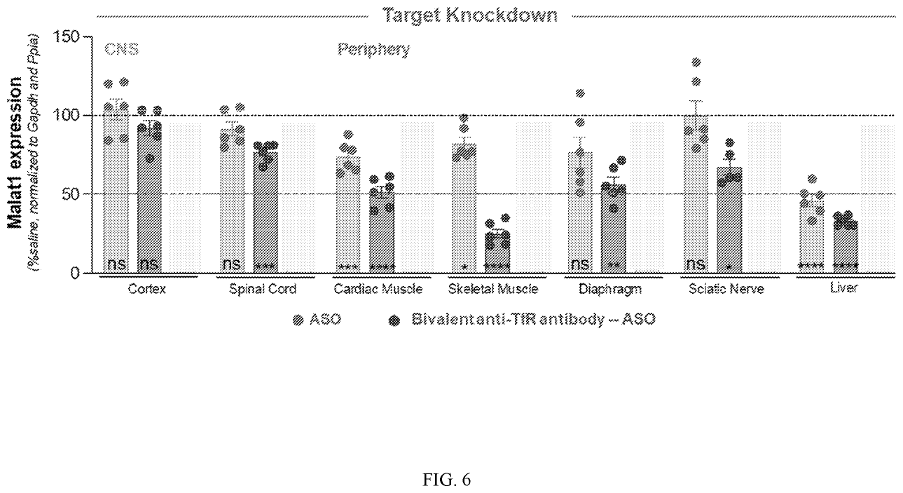

Results are shown in FIG. 6. Some Malat1 knockdown is observed in the CNS and higher Malat1 knockdown is observed in the periphery.

### Example 4: In Vivo Pharmacokinetics and Biodistribution Using TfR Mono-Fab Conjugates

Two TfR-mono Fab conjugates were generated in Example 1 (TfR mono-Fab and TfR mono-Fab 2) and were diluted in sterile saline before administration. TfR mono-Fab conjugate has a TfR binding arm and a non-binding RSV arm and is conjugated to mouse MALAT1 (SEQ ID NO: 8) (anti-TfR/non-binding Fab antibody-oligonucleotide) and TfR-mono-Fab 2 conjugate has a TfR binding arm (second arm is absent; mono-Fab) and is conjugated to cyno MALAT1 (SEQ ID NO: 172). Unconjugated ASO was administered as a control. Two-month-old TfR ms/hu female mice were given intravenous doses of either naked ASO (0.9 mg per kg (mpk)), TfR-mono Fab conjugate (25 mpk), or TfR-mono Fab 2 conjugate (17.2 mpk). The following tissues were collected 24 hours after the single dose: brain, spinal cord, kidney, diaphragm, liver, and quadricep muscle. Plasma was also collected 15 min, 4 hr, and 24 hr after the single dose.

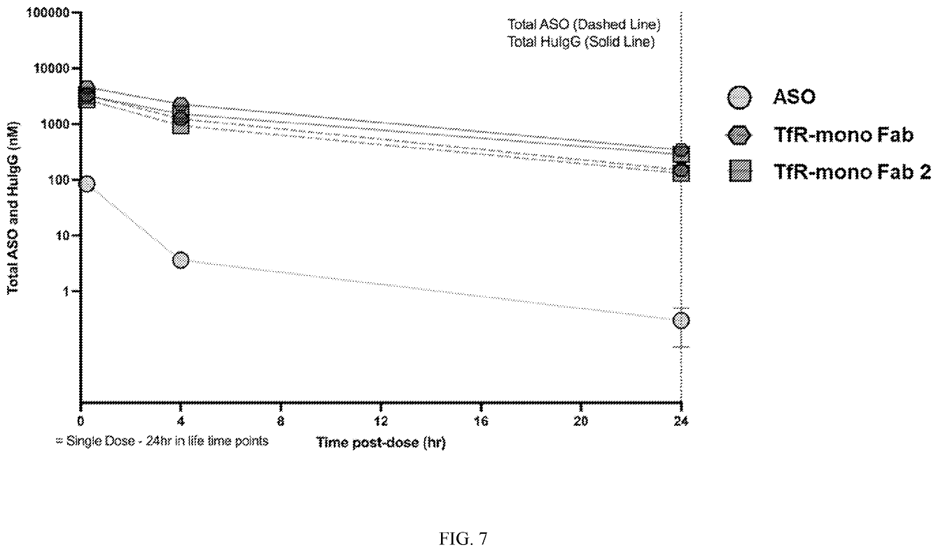

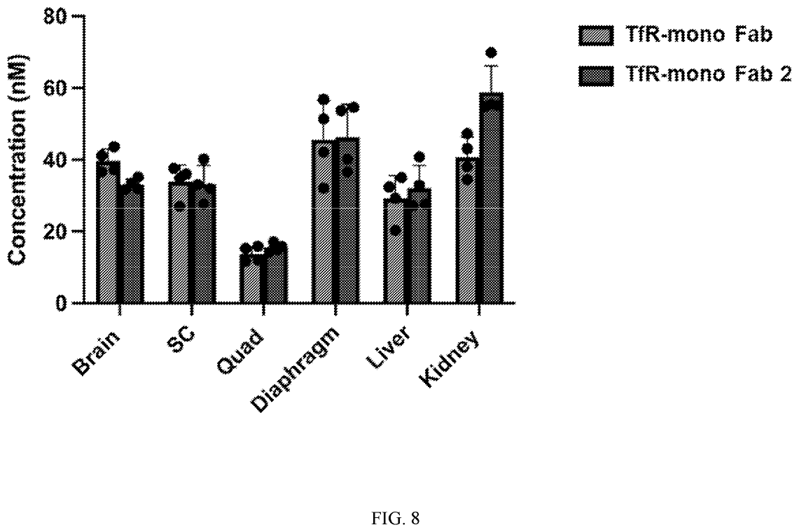

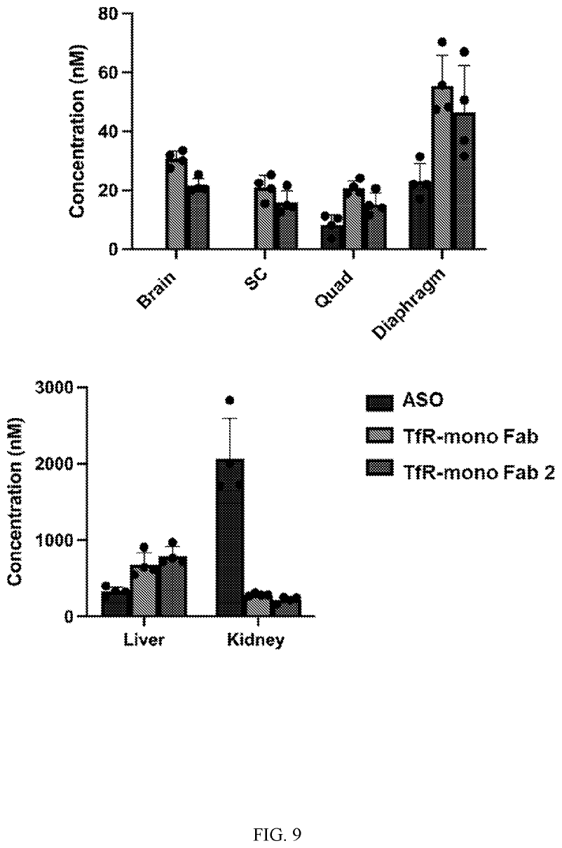

Total ASO and total huIgG were measured according to the methods described above in Example 2. Results are shown in FIGS. 7-9. The two TfR-mono Fab conjugate molecules display similar pharmacokinetic profiles in plasma (FIG. 7) and similar patterns of biodistribution throughout the body (FIG. 8). Compared to unconjugated ASO, which clears quickly from circulation and is not detected in the brain or spinal cord after a 0.9 mpk dose, molar equivalent amounts of both TfR-mono Fab molecules achieve robust CNS ASO uptake (FIG. 9). Additionally, both TfR-mono Fab molecules result in more ASO in the diaphragm, quadricep muscle, and liver, but significantly less ASO in the kidney (FIG. 9).

### Example 5: TfR-Albumin-ASO Construction

TfR-Albumin-ASO molecule was generated by fusing TfR binding scFv to mouse serum albumin via a linker. A linker-ASO was also generated using the linker shown in Example 1 and mouse MALAT1 (SEQ ID NO:8). The cysteine at position 34 (of SEQ ID NO: 168) was used for conjugation purposes. For bioconjugation of the linker-ASO to the TfR-albumin protein, the TfR-albumin protein was first reduced using a TCEP (30 molar equivalent). The linker-ASO was then conjugated to the free cysteine on the TfR-albumin protein (1.2 molar equivalent). The resulting conjugate was purified by cation exchange chromatography (Mobile Phase A: 20 mM Sodium Acetate, pH 5 Mobile Phase B1: 20 mm Sodium Acetate, 1M NaCl, pH 5) to remove unwanted and unconjugated products and the purity was determined by LC/MS and analytical SEC.

### Example 6: In Vivo Pharmacokinetics and Biodistribution Using TfR-Albumin-ASO Conjugate

The TfR-albumin-ASO molecule prepared above was diluted in sterile saline before administration. As a control unconjugated ASO was also dosed.

4-8mo old TfR^{ms/hu} female mice were administered doses intravenously. TfR-albumin-ASO was dosed at 9.5 mg/kg (n=2) Unconjugated ASO (“naked ASO”) was dosed at 1.37 mg/kg (n=3). Plasma collections were taken at 15 min (unconjugated ASO only), 4 hours, 24 hours, after dosing. Tissues including brain, liver and kidney, as well as terminal plasma were collected 72 hours after dosing. ASO concentrations were measured as described in Example 2.

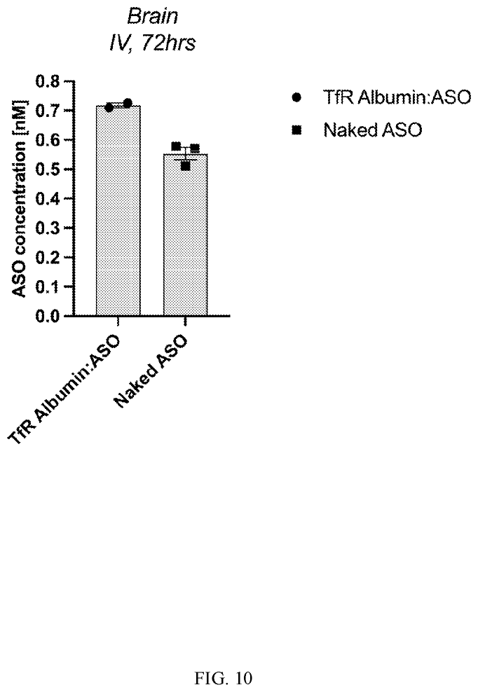

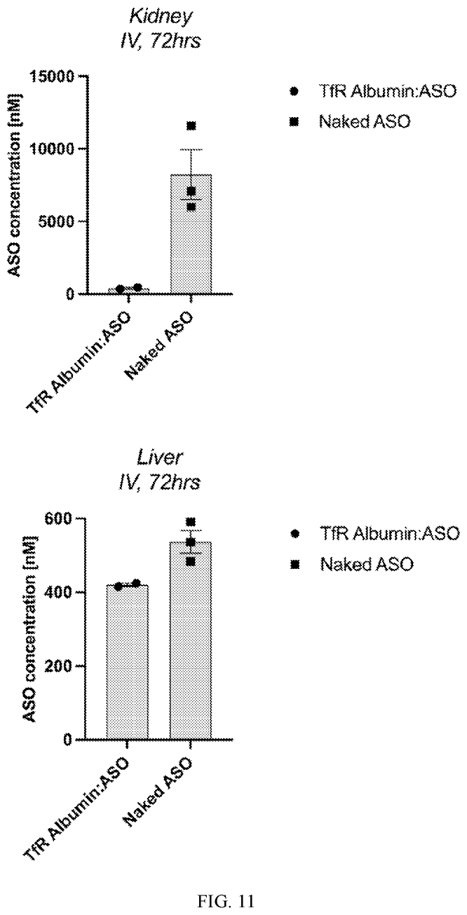

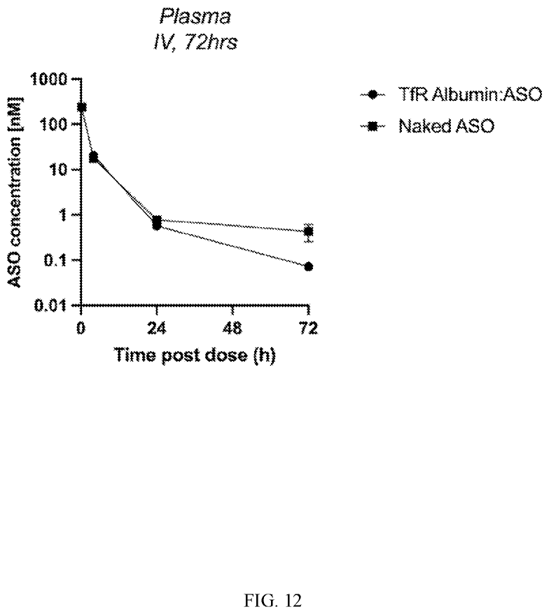

Results are shown in FIGS. 10-12. Increased delivery of ASO to the brain is observed with the TfR-albumin-ASO conjugate compared to naked ASO (FIG. 10). The liver and kidneys are a sink for naked ASO, with concentrations of ASO in these organs reduced with the TfR-albumin-ASO conjugate (FIG. 11). In plasma, clearance is similar between the two molecules with slightly accelerated clearance for the TfR-albumin-ASO conjugate (FIG. 12).

Many modifications and other embodiments of the subject matter set forth herein will come to mind to one skilled in the art to which the inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A TfR binder-oligonucleotide conjugate comprising: 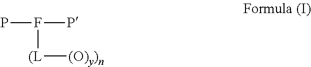
wherein
P comprises an anti-TfR antibody antigen binding domain,
F is present or absent, and if present comprises a peptide, a Fc polypeptide, a Fc dimer, or albumin;
L is present or absent, and if present comprises a linking group;
P′ is present or absent, and if present comprises an anti-TfR antibody antigen binding domain or a non-binding Fab or a non-binding variable region (NBVR);
O comprises an oligonucleotide;
y is an integer greater than or equal to 1 (e.g., 1, 2, 3, or 4); and
n is an integer greater than or equal to 1 (e.g., 1, 2, 3, 4, 5, 6, 7 or 8).

2. The TfR binder-oligonucleotide conjugate of claim 1, wherein
F is a Fc dimer; and
P′ is absent.

3. The TfR binder-oligonucleotide conjugate of claim 2, wherein
P is an anti-TfR Fab, a single chain Fab (scFab), or a single chain variable fragment (scFv);

4. The TfR binder-oligonucleotide conjugate of claim 3, wherein
P is an anti-TfR Fab or a scFab.

5. The TfR binder-oligonucleotide conjugate of claim 1, wherein
F is a Fc dimer; and
P′ is a non-binding Fab or a NBVR.

6. The TfR binder-oligonucleotide conjugate of claim 5, wherein
P is an anti-TfR Fab, a single chain Fab (scFab), or a single chain variable fragment (scFv);

7. The TfR binder-oligonucleotide conjugate of claim 6, wherein
P is an anti-TfR Fab or a scFab.

8. The TfR binder-oligonucleotide conjugate of claim 1, wherein
F is an albumin; and
P′ is absent.

9. The TfR binder-oligonucleotide conjugate of claim 8, wherein
P is a scFv or a nanobody.

10. The TfR binder-oligonucleotide conjugate of claim 9, wherein
P is a scFv.

11. The TfR binder-oligonucleotide conjugate of claim 1, wherein
F is Fc dimer; and
P′ is an anti-TfR antibody antigen binding domain.

12. The TfR binder-oligonucleotide conjugate of claim 11, wherein
P-F-P′ comprises an antibody.

13. The TfR binder-oligonucleotide conjugate of claim 12, wherein the antibody comprises an divalent anti-TfR antibody or a bispecific antibody.

14. The TfR binder-oligonucleotide conjugate of claim 11, wherein
P is an anti-TfR Fab, a single chain Fab (scFab), a single chain variable fragment (scFv), or a nanobody.

15. The TfR binder-oligonucleotide conjugate of claim 14, wherein
P is an anti-TfR Fab or a scFab.

16. The TfR binder-oligonucleotide conjugate of claim 1, wherein
P is a first Fab known to specifically bind TfR; and
Fis Fc dimer,
wherein P-F comprises a modification for covalent conjugation; and, wherein O is conjugated to P-F at the site of the modification.

17. The conjugate of claim 16, further comprising a second Fab that is a non-targeting Fab or does not bind TfR.

18. The TfR binder-oligonucleotide conjugate of any one of claims 1-17, wherein P comprises three heavy chain CDRs and three light chain CDRs wherein:
(a) CDR-H1 comprises SEQ ID NO: 12,
CDR-H2 comprises SEQ ID NO: 13,
CDR-H3 comprises SEQ ID NO: 14,
CDR-L1 comprises SEQ ID NO: 15,
CDR-L2 comprises SEQ ID NO: 16, and
CDR-L3 comprises SEQ ID NO: 17;
(b) CDR-H1 comprises SEQ ID NO: 21,
CDR-H2 comprises SEQ ID NO: 22,
CDR-H3 comprises SEQ ID NO: 23,
CDR-L1 comprises SEQ ID NO: 24,
CDR-L2 comprises SEQ ID NO: 25, and
CDR-L3 comprises SEQ ID NO: 26;
(c) CDR-H1 comprises SEQ ID NO: 114,
CDR-H2 comprises SEQ ID NO: 115,
CDR-H3 comprises SEQ ID NO: 116,
CDR-L1 comprises SEQ ID NO: 117,
CDR-L2 comprises SEQ ID NO: 118, and
CDR-L3 comprises SEQ ID NO: 119;
(d) CDR-H1 comprises SEQ ID NO: 126,
CDR-H2 comprises SEQ ID NO: 127,
CDR-H3 comprises SEQ ID NO: 128,
CDR-L1 comprises SEQ ID NO: 129,
CDR-L2 comprises SEQ ID NO: 130, and
CDR-L3 comprises SEQ ID NO: 131;
(e) CDR-H1 comprises SEQ ID NO: 134,
CDR-H2 comprises SEQ ID NO: 135,
CDR-H3 comprises SEQ ID NO: 136,
CDR-L1 comprises SEQ ID NO: 137,
CDR-L2 comprises SEQ ID NO: 138, and
CDR-L3 comprises SEQ ID NO: 139;
(f) CDR-H1 comprises SEQ ID NO: 154,
CDR-H2 comprises SEQ ID NO: 155,
CDR-H3 comprises SEQ ID NO: 156,
CDR-L1 comprises SEQ ID NO: 157,
CDR-L2 comprises SEQ ID NO: 158, and
CDR-L3 comprises SEQ ID NO: 159; or
(g) CDR-H1 comprises SEQ ID NO: 161,
CDR-H2 comprises SEQ ID NO: 162,
CDR-H3 comprises SEQ ID NO: 163,
CDR-L1 comprises SEQ ID NO: 164,
CDR-L2 comprises SEQ ID NO: 165, and
CDR-L3 comprises SEQ ID NO: 166.

19. The TfR binder-oligonucleotide conjugate of any one of claims 1-8 and 11-17,
wherein P is an anti-TfR Fab comprising:
(a) SEQ ID NOs: 102 and 103;
(b) SEQ ID NOs: 104 and 105;
(c) SEQ ID NOs: 110 and 111;
(d) SEQ ID NOs: 122 and 123;
(e) SEQ ID NOs: 132 and 133; or
(f) SEQ ID NOs: 143 and 144.
optionally wherein P comprises one or more modifications for covalent conjugation of the oligonucleotide.

20. The TfR binder-oligonucleotide conjugate of any one of claims 1-3, 5-6, 8-10, 11, 14, and 16-17, wherein P is a scFv comprising:
(a) SEQ ID NO: 106
(b) SEQ ID NO: 107
(c) SEQ ID NO: 171
(d) SEQ ID NO: 153
(e) SEQ ID NO: 160
(f) SEQ ID NOs: 112 and 113
(g) SEQ ID NOs: 124 and 125
(h) SEQ ID NOs: 145 and 146

21. The TfR binder-oligonucleotide conjugate of any one of claims 1-3, 5-6, 8-10, 11, 14, and 16-17, wherein P is an antibody comprising:
(a) SEQ ID NOs: 108 and 109;
(b) SEQ ID NOs: 120 and 121;
(c) SEQ ID NOs: 9, 10, and 11; or
(d) SEQ ID NOs: 18, 19;
optionally wherein the antibody contains one or more modifications to increase serum stability, modulate effector function, influence glycosylation, reduce immunogenicity in humans, facilitate heterodimerization, and/or facilitate conjugation of the oligonucleotide.

22. The TfR binder-oligonucleotide of any one of claims 1-21, wherein P, F, or P′ comprises a modification for conjugation of the oligonucleotide.

23. The TfR binder-oligonucleotide conjugate claim 22, wherein the modification for conjugation is a cysteine modification.

24. The TfR binder-oligonucleotide conjugate of claim 23, wherein F comprises the Fc polypeptide or the Fc dimer and wherein the cysteine modification is a S239C, S442C, A330C, or T289C substitution, wherein the position is according to EU numbering.

25. The TfR binder-oligonucleotide conjugate of claim 24, wherein the cysteine modification is the S239C substitution.

26. The TfR binder-oligonucleotide conjugate of claim 23, wherein P comprises a Fab or scFab and wherein the cysteine modification comprises a K149C substitution, wherein the position is according to EU numbering, or a A114C substitution, wherein the position is according to Kabat numbering.

27. The TfR binder-oligonucleotide conjugate of claim 22, wherein F comprises a Fc polypeptide or a Fc dimer and wherein the modification comprises a N297A substitution or a N297G substitution, wherein the position is according to EU numbering.

28. The TfR binder-oligonucleotide conjugate of any one of claims 1-7 and 11-27, wherein F comprises a Fc polypeptide or a Fc dimer and wherein the Fc polypeptide or one or both Fc polypeptides of the Fc dimer contains at least one modification to increase serum stability, modulate effector function, influence glycosylation, reduce immunogenicity in humans, and/or facilitate heterodimerization.

29. The TfR binder-oligonucleotide conjugate of claim 28, wherein the at least one modification comprises:
(a) a L234A substitution and a L235A substitution;
(b) a P329G substitution; or
(c) a L234A substitution, a L235A substitution, and a P329G substitution;
wherein the positions is according to EU numbering.

30. The TfR binder-oligonucleotide conjugate of claim 28 or 29, wherein the at least one modification comprises:
(a) a M428L substitution and a N434S substitution;
(b) a M428L substitution;
(b) a N434S substitutions;
(c) a N434A substitution; or
(d) a M252Y substitution, a S254T substitution, and a T256E substitution;
wherein the positions is according to EU numbering.

31. The TfR binder-oligonucleotide conjugate of any one of claims 28-30, wherein the at least one modification comprises: a deletion of a carboxy terminal lysine.

32. The TfR binder-oligonucleotide conjugate of any one of claims 28-31, wherein the at least one modification comprises:
(a) a M428L substitution and a N434S substitution;
(b) a M428L substitution;
(b) a N434S substitutions;
(c) a N434A substitution; or
(d) a M252Y substitution, a S254T substitution, and a T256E substitution;
wherein the positions is according to EU numbering.

33. The TfR binder-oligonucleotide conjugate of any one or claims 28-32, wherein F comprises the Fc dimer, and wherein a first Fc polypeptide of the Fc dimer comprises a knob mutation and a second Fc polypeptide of the Fc dimer comprises a hole mutation.

34. The TfR binder-oligonucleotide conjugate of claim 33, wherein the knob mutation comprises a T366W substitution, according to EU numbering scheme; and the hole mutation comprises a Y407V substitution and optionally a T366S substitution and a L368A substitution, according to EU numbering scheme.

35. The TfR binder-oligonucleotide conjugate of any one of claims 1, 5-7, 11-34, wherein P′ is present and comprises a non-binding Fab or NBVR, wherein the non-binding Fab or NBVR comprises three heavy chain CDRs and three light chain CDRs wherein:
(a) CDR-H1 comprises SEQ ID NO: 45,
CDR-H2 comprises SEQ ID NO: 47,
CDR-H3 comprises SEQ ID NO: 49,
CDR-L1 comprises SEQ ID NO: 39;
CDR-L2 comprises SEQ ID NO: 41, and
CDR-L3 comprises SEQ ID NO: 43;
(b) CDR-H1 comprises SEQ ID NO: 46,
CDR-H2 comprises SEQ ID NO: 48,
CDR-H3 comprises SEQ ID NO: 49,
CDR-L1 comprises SEQ ID NO: 40;
CDR-L2 comprises SEQ ID NO: 42, and
CDR-L3 comprises SEQ ID NO: 44; or
(c) CDR-H1 comprises SEQ ID NO: 46,
CDR-H2 comprises SEQ ID NO: 50,
CDR-H3 comprises SEQ ID NO: 49,
CDR-L1 comprises SEQ ID NO: 40;
CDR-L2 comprises SEQ ID NO: 42, and
CDR-L3 comprises SEQ ID NO: 44; or

36. The TfR binder-oligonucleotide conjugate of claim 35, wherein P′ comprises:
(a) SEQ ID NO: 35 and SEQ ID NO: 36;
(b) SEQ ID NO: 37 or 52 and SEQ ID NO: 38;
(c) SEQ ID NO: 37 or 52 and SEQ ID NO: 53;
(d) SEQ ID NO: 37 or 52 and SEQ ID NO: 54;
(e) SEQ ID NO: 37 or 52 and SEQ ID NO: 55;
(f) SEQ ID NO: 37 or 52 and SEQ ID NO: 56;
(g) SEQ ID NO: 37 or 52 and SEQ ID NO: 57;
(h) SEQ ID NO: 37 or 52 and SEQ ID NO: 58;
(i) SEQ ID NO: 37 or 52 and SEQ ID NO: 59;
(j) SEQ ID NO: 51 and SEQ ID NO: 60;
(k) SEQ ID NO: 62 or 64 and SEQ ID NO: 61;
(l) SEQ ID NO: 62 or 64 and SEQ ID NO: 65;
(m) SEQ ID NO: 62 or 64 and SEQ ID NO: 66;
(n) SEQ ID NO: 62 or 64 and SEQ ID NO: 67;
(o) SEQ ID NO: 62 or 64 and SEQ ID NO: 68;
(p) SEQ ID NO: 62 or 64 and SEQ ID NO: 69;
(q) SEQ ID NO: 62 or 64 and SEQ ID NO: 70; or
(r) SEQ ID NO: 62 or 64 and SEQ ID NO: 71.

37. The conjugate of any one of claims 1-36, wherein the oligonucleotide is a single-stranded oligonucleotide.

38. The conjugate of claim 37, wherein the oligonucleotide is an antisense oligonucleotide.

39. The conjugate of any one of claims 22-36, wherein the oligonucleotide is conjugated to the site of the modification through the linking group.

40. The TfR binder-oligonucleotide conjugate of claim 1 comprising:
a protein comprising:
a Fc dimer,
a first Fab known to specifically bind TfR, and
a S239C substitution, wherein the position is according to EU numbering; and,
an oligonucleotide conjugated to the protein at the S239C substitution.

41. A method of modulating the expression of a target gene in a muscle cell of a subject comprising administering to the subject the TfR binder-oligonucleotide conjugate of any one of claims 1-40.

42. The method of claim 41, wherein the muscle cell is a cardiac muscle cell or a skeletal muscle cell.

43. A method of delivering an oligonucleotide to the central nervous system (CNS) of a subject comprising administering to the subject the TfR binder-oligonucleotide conjugate of any one of claims 1-40.

44. A method of delivering an oligonucleotide to a cancer of a subject comprising administering to the subject the TfR binder-oligonucleotide conjugate of any one of claims 1-40.

45. An TfR binder-oligonucleotide conjugate comprising:
an antibody that specifically binds to TfR, wherein the antibody comprises three heavy chain CDRs and three light chain CDRs wherein:
a) CDR-H1 comprises SEQ ID NO: 12,
CDR-H2 comprises SEQ ID NO: 13,
CDR-H3 comprises SEQ ID NO: 14,
CDR-L1 comprises SEQ ID NO: 15,
CDR-L2 comprises SEQ ID NO: 16, and
CDR-L3 comprises SEQ ID NO: 17;
b) CDR-H1 comprises SEQ ID NO: 21,
CDR-H2 comprises SEQ ID NO: 22,
CDR-H3 comprises SEQ ID NO: 23,
CDR-L1 comprises SEQ ID NO: 24,
CDR-L2 comprises SEQ ID NO: 25, and
CDR-L3 comprises SEQ ID NO: 26;
(c) CDR-H1 comprises SEQ ID NO: 114,
CDR-H2 comprises SEQ ID NO: 115,
CDR-H3 comprises SEQ ID NO: 116,
CDR-L1 comprises SEQ ID NO: 117,
CDR-L2 comprises SEQ ID NO: 118, and
CDR-L3 comprises SEQ ID NO: 119;
(d) CDR-H1 comprises SEQ ID NO: 126,
CDR-H2 comprises SEQ ID NO: 127,
CDR-H3 comprises SEQ ID NO: 128,
CDR-L1 comprises SEQ ID NO: 129,
CDR-L2 comprises SEQ ID NO: 130, and
CDR-L3 comprises SEQ ID NO: 131;
(e) CDR-H1 comprises SEQ ID NO: 134,
CDR-H2 comprises SEQ ID NO: 135,
CDR-H3 comprises SEQ ID NO: 136,
CDR-L1 comprises SEQ ID NO: 137,
CDR-L2 comprises SEQ ID NO: 138, and
CDR-L3 comprises SEQ ID NO: 139;
(f) CDR-H1 comprises SEQ ID NO: 154,
CDR-H2 comprises SEQ ID NO: 155,
CDR-H3 comprises SEQ ID NO: 156,
CDR-L1 comprises SEQ ID NO: 157,
CDR-L2 comprises SEQ ID NO: 158, and
CDR-L3 comprises SEQ ID NO: 159; or
(g) CDR-H1 comprises SEQ ID NO: 161,
CDR-H2 comprises SEQ ID NO: 162,
CDR-H3 comprises SEQ ID NO: 163,
CDR-L1 comprises SEQ ID NO: 164,
CDR-L2 comprises SEQ ID NO: 165, and
CDR-L3 comprises SEQ ID NO: 166; and
an oligonucleotide conjugated to a cysteine modification on the constant domain of the antibody.

46. The TfR binder-oligonucleotide conjugate of claim 45, wherein the cysteine modification is a S239C, S442C, A330C, K149C, or T289C substitution, wherein the position is according to EU numbering, or a A114C substitution, wherein the position is according to Kabat numbering.

47. The TfR binder-oligonucleotide conjugate of claim 46, wherein the cysteine modification is the S239C substitution.

48. The TfR binder-oligonucleotide conjugate of any one of claims 45-47, wherein the oligonucleotide is a single-stranded oligonucleotide.

49. The TfR binder-oligonucleotide conjugate of claim 48, wherein the oligonucleotide is an antisense oligonucleotide.

50. The TfR binder-oligonucleotide conjugate comprising:
an antibody that specifically binds TfR, wherein the antibody comprises three heavy chain CDRs and three light chain CDRs wherein
a) CDR-H1 comprises SEQ ID NO: 12,
CDR-H2 comprises SEQ ID NO: 13,
CDR-H3 comprises SEQ ID NO: 14,
CDR-L1 comprises SEQ ID NO: 15,
CDR-L2 comprises SEQ ID NO: 16, and
CDR-L3 comprises SEQ ID NO: 17;
b) CDR-H1 comprises SEQ ID NO: 21,
CDR-H2 comprises SEQ ID NO: 22,
CDR-H3 comprises SEQ ID NO: 23,
CDR-L1 comprises SEQ ID NO: 24,
CDR-L2 comprises SEQ ID NO: 25, and
CDR-L3 comprises SEQ ID NO: 26;
(c) CDR-H1 comprises SEQ ID NO: 114,
CDR-H2 comprises SEQ ID NO: 115,
CDR-H3 comprises SEQ ID NO: 116,
CDR-L1 comprises SEQ ID NO: 117,
CDR-L2 comprises SEQ ID NO: 118, and
CDR-L3 comprises SEQ ID NO: 119;
(d) CDR-H1 comprises SEQ ID NO: 126,
CDR-H2 comprises SEQ ID NO: 127,
CDR-H3 comprises SEQ ID NO: 128,
CDR-L1 comprises SEQ ID NO: 129,
CDR-L2 comprises SEQ ID NO: 130, and
CDR-L3 comprises SEQ ID NO: 131;
(e) CDR-H1 comprises SEQ ID NO: 134,
CDR-H2 comprises SEQ ID NO: 135,
CDR-H3 comprises SEQ ID NO: 136,
CDR-L1 comprises SEQ ID NO: 137,
CDR-L2 comprises SEQ ID NO: 138, and
CDR-L3 comprises SEQ ID NO: 139;
(f) CDR-H1 comprises SEQ ID NO: 154,
CDR-H2 comprises SEQ ID NO: 155,
CDR-H3 comprises SEQ ID NO: 156,
CDR-L1 comprises SEQ ID NO: 157,
CDR-L2 comprises SEQ ID NO: 158, and
CDR-L3 comprises SEQ ID NO: 159; or
(g) CDR-H1 comprises SEQ ID NO: 161,
CDR-H2 comprises SEQ ID NO: 162,
CDR-H3 comprises SEQ ID NO: 163,
CDR-L1 comprises SEQ ID NO: 164,
CDR-L2 comprises SEQ ID NO: 165, and
CDR-L3 comprises SEQ ID NO: 166; and
an oligonucleotide conjugated through a linker to S239C substitution on the constant domain of the antibody, wherein the position is according to EU numbering.

51. A method of modulating the expression of a target gene in the muscle cell of a subject comprising administering to the subject the TfR binder-oligonucleotide conjugate of any one of claims 45-50.

52. The method of claim 51, wherein the muscle cell is cardiac muscle cell or a skeletal muscle cell.

53. A method of delivering an oligonucleotide to the CNS of a subject comprising administering to the subject the TfR binder-oligonucleotide conjugate of any one of claims 45-50.

54. A method of delivering an oligonucleotide to a cancer cell of a subject comprising administering to the subject the TfR binder-oligonucleotide conjugate of any one of claims 45-50.
